(19) United States
(12) Patent Application Publication
(10) Pub. No.: US 2023/0406934 A1
(43) Pub. Date: Dec. 21, 2023

(54) UNGULATE-DERIVED POLYCLONAL IMMUNOGLOBULIN SPECIFIC FOR PD-LI AND USES THEREOF

(71) Applicant: SAB, LLC, Sioux Falls, SD (US)

(72) Inventors: Christoph L. BAUSCH, Sioux Falls, SD (US); Hua Wu, Sioux Falls, SD (US); Kristi A. EGLAND, Sioux Falls, SD (US); Eddie J. SULLIVAN, Sioux Falls, SD (US)

(73) Assignee: SAB, LLC, Sioux Falls, SD (US)

(21) Appl. No.: 18/036,567

(22) PCT Filed: Nov. 12, 2021
(86) PCT No.: PCT/US2021/059262

§ 371 Date: May 11, 2023

Related U.S. Application Data
(60) Provisional application No. 63/113,635, filed on Nov. 13, 2020.

(51) Int. Cl. C07K 16/28; A61P 35/00

(52) U.S. Cl. CPC C07K 16/2827; A61P 35/00; C07K 2317/20; C07K 2317/732; C07K 2317/76; C07K 2317/734; A61K 2039/505

(57) ABSTRACT

Provided are human polyclonal immunoglobulin products specific for Programmed Death-Ligand 1 (PD-L1) for use in treating or preventing cancer. Further provided are methods for making such compositions in a transgenic ungulate, e.g. using a transchromosomic bovine (TcB) system.

### CROSS-REFERENCE TO RELATED APPLICATIONS

This application claims priority to U.S. Application No. 63/113,635, filed Nov. 13, 2020, which is hereby incorporated by reference in its entirety herein.

### SEQUENCE LISTING

This application contains a Sequence Listing which has been submitted in ASCII format via EFS-WEB and is hereby incorporated by reference in its entirety. Said ASCII copy, created on Nov. 11, 2021, is named SABB_003_01WO_ST25.txt and is 80 kilobytes in size.

### TECHNICAL FIELD

The invention relates to polyclonal immunoglobulin products for treatment of cancer.

### BACKGROUND

For over half a century, physicians have relied on surgery, chemotherapy, and radiotherapy as the main weapons to fight cancer. Recently, new immunotherapies have been added to the arsenal and are quickly becoming routine therapy for cancer patients. These immune-oncology agents are antagonistic antibodies that block specific immune checkpoints to restore the immune response against the cancer cells. Immune checkpoints are inhibitory pathways induced in activated T cells that are crucial for maintaining self-tolerance and regulating the extent of the immune response to minimize peripheral tissue damage. Cytotoxic T-Lymphocyte-Associated Antigen 4 (CTLA-4) and Programmed Cell Death protein (PD-1) with its ligands PD-L1/L2 are immune checkpoint regulatory molecules and are members of the B7 family of immune-regulatory ligands. CTLA-4 is present on the surface of CD4+ and CD8+ T cells and regulates the amplitude and duration of T cell activation during adaptive immune responses.

PD-1 encodes a type I transmembrane protein that binds to two ligands: PD-L1, present on most tissues including tumors, and PD-L2, found on macrophages and dendritic cells. The primary role of PD-1 is to limit the response of T cells during an inflammatory response in peripheral tissues. PD-1 is expressed in activated effector T cells, B cells, natural killer (NK) cells and regulatory T (Treg) cells. The PD-1 pathway suppresses effector T cell function, B cell antibody production, as well as the lytic capacity of NK cells. In addition, PD-L1 promotes the development and sustains the function of induced Treg cells, which maintains the immune suppressive microenvironment of the tumor.

Antagonistic monoclonal antibodies (mAbs) against CTLA-4 can be used to block the inhibitory signals and enhance T cell activation leading to tumor regression. A fully human mAb against CTLA-4, ipilimumab, was the first immune checkpoint inhibitor that demonstrated activity in patients with metastatic melanoma. Following, humanized antagonistic mAbs against PD-1, pembrolizumab and nivolumab, were tested in melanoma patients. In addition, monoclonal antibodies against PD-L1, such as atezolizumab, avelumab and durvalumab, are being investigated.

There exists a need for immunoglobulin products for therapeutic use in patients suffering from or at risk for diseases and disorders including but not limited to cancer.

### SUMMARY

The present inventors have developed a polyclonal human immunoglobulin product for treatment of disease associated with the PD-L1/PD-1 axis made in ungulates (e.g., bovines) genetically engineered to produce polyclonal human immunoglobulin having a human polypeptide sequence, representative samples of which were deposited under the Budapest treaty on Nov. 2, 2021, with the American Type Culture Collection (ATCC) and given the Deposit No. PTA-127159. An anti-PD-L1 human polyclonal product may have substantial therapeutic and safety benefits compared to monoclonal antibody therapy.

In one aspect, the disclosure provides an ungulate-derived polyclonal human immunoglobulin composition, comprising a population of human immunoglobulins, wherein the population of human immunoglobulins specifically binds Programmed Death-Ligand 1 (PD-L1).

In some embodiments, the composition is produced by immunizing a transgenic ungulate with an antigenic fragment of PD-L1.

In some embodiments, the antigenic fragment of PD-L1 is a PD-L1 extracellular domain. In some embodiments, the antigenic fragment comprises, consists of, or consists essentially of amino acids 19-238 of a PD-L1 sequence according to SEQ ID NO: 15 or a variant thereof.

In some embodiments, the antigenic fragment shares at least 80%, 90%, 95%, 96%, 97%, 98%, 99%, or 100% identity to SEQ ID NO: 15 or a fragment thereof.

In some embodiments, the population of human immunoglobulins binds MC38-hPD-L1 cells with at least as high affinity, or higher affinity than atezolizumab.

In some embodiments, the population of human immunoglobulins binds to a non-small cell lung cancer cell, optionally one or more of H292, H460, H1975, HCC827, and H1299 cells.

In some embodiments, the population of human immunoglobulins exhibit complement-dependent-cytotoxicity (CDC) activity, optionally at an IC₅₀ of about 0.72 μg/mL or less.

In some embodiments, the population of human immunoglobulins inhibits tumor cell growth in vivo.

In some embodiments, the population of human immunoglobulins exhibit antibody-dependent cellular toxicity (ADCC) activity.

In some embodiments, the population of human immunoglobulins block PD-L1 from binding to the PD-1 receptor.

In some embodiments, the population of human immunoglobulins blocks the PD-1 signaling pathway.

In some embodiments, the population of human immunoglobulin enhances effector cell function, optionally natural killer cells.

In some embodiments, the population of human immunoglobulins has an avidity for PD-L1 of at least 0.1 1/sec, at least 0.01 1/sec, at least 0.001 1/sec at least 0.0001 1/sec, or at least 0.00001 1/sec.

In some embodiments, the population of human immunoglobulins has an avidity for PD-L1 of 0.1 to 0.01 1/sec, 0.01 to 0.001 1/sec, 0.001 to 0.0001 1/sec, or 0.0001 to 0.00001 1/sec.

In some embodiments, the population of human immunoglobulin composition is substantially similar to ATCC Deposit No. PTA-127159 or wherein population of human immunoglobulins has an avidity for PD-L1 at least 50%, at least 60%, at least 70%, at least 80%, at least 90%, at least 100%, at least 110%, or at least 120% that of ATCC Deposit No. PTA-127159.

In another aspect, the disclosure provides a method of making polyclonal human immunoglobulin specific for Programmed Death-Ligand 1 (PD-L1), comprising administering an antigenic fragment of PD-L1, or a polynucleotide encoding the antigenic fragment, to a transgenic ungulate, wherein the transgenic ungulate comprises a genome comprising a human immunoglobulin locus or an artificial chromosome comprising a human immunoglobulin locus, and wherein the transgenic ungulate produces a population of human immunoglobulins that specifically binds PD-L1.

In some embodiments, wherein the method comprises administering the antigenic fragment or polynucleotide encoding the antigenic fragment 3, 4, 5, or more times.

In some embodiments, wherein the method comprises collecting serum or plasma from the transgenic ungulate.

In some embodiments, the serum or plasma comprises a population of fully human immunoglobulins.

In some embodiments, the antigenic fragment of PD-L1 is a PD-L1 extracellular domain.

In some embodiments, the antigenic fragment comprises, consists of, or consists essentially of amino acids 19-238 of a PD-L1 sequence according to SEQ ID NO: 15 or a variant thereof.

In some embodiments, the antigenic fragment shares at least 80%, 90%, 95%, 96%, 97%, 98%, 99%, or 100% identity to SEQ ID NO: 15 or a fragment thereof.

In some embodiments, the population of human immunoglobulins binds MC38-hPD-L1 cells with at least as high affinity, or higher affinity than atezolizumab.

In some embodiments, the population of human immunoglobulins binds to a non-small cell lung cancer cell, optionally one or more of H292, H460, H1975, HCC827, and H1299 cells.

In some embodiments, the population of human immunoglobulins exhibit complement-dependent-cytotoxicity (CDC) activity, optionally at an IC₅₀ of about 0.72 μg/mL or less.

In some embodiments, the population of human immunoglobulins inhibits tumor cell growth in vivo.

In some embodiments, the antigenic fragment is administering in a pharmaceutical composition comprising Montanide ISA-206 and/or Quil A.

In some embodiments, the method comprises a) administering a polynucleotide encoding the antigenic fragment of PD-L1; b) administering a polynucleotide encoding the antigenic fragment of PD-L1, three to four weeks later; c) administering the antigenic fragment of PD-L1, four weeks later; d) administering the antigenic fragment of PD-L1, four weeks later; and e) administering the antigenic fragment of PD-L1, four weeks later.

In some embodiments, wherein the method comprises purifying the human immunoglobulin to produce a composition according to the disclosure.

In some embodiments, the population of human immunoglobulins exhibit antibody-dependent cellular toxicity (ADCC) activity.

In some embodiments, wherein the population of human immunoglobulins block PD-L1 from binding to PD-1.

In some embodiments, the population of human immunoglobulins blocks the PD-1 signaling pathway.

In some embodiments, the population of human immunoglobulin enhances effector cell function, such as natural killer cells. In some embodiments, the population of human immunoglobulins has an avidity for PD-L1 of at least 0.1 1/sec, at least 0.01 1/sec, at least 0.001 1/sec at least 0.0001 1/sec, or at least 0.00001 1/sec.

In some embodiments, the population of human immunoglobulins has an avidity for PD-L1 of 0.1 to 0.01 1/sec, 0.01 to 0.001 1/sec, 0.001 to 0.0001 1/sec, or 0.0001 to 0.00001 1/sec.

In some embodiments, the population of human immunoglobulin composition is substantially similar to ATCC Deposit No. PTA-127159 or wherein population of human immunoglobulins has an avidity for PD-L1 at least 50%, at least 60%, at least 70%, at least 80%, at least 90%, at least 100%, at least 110%, or at least 120% that of ATCC Deposit No. PTA-127159.

In another aspect, the disclosure provides a pharmaceutical composition, comprising a composition of the disclosure and optionally one or more pharmaceutically acceptable excipients.

In some embodiments, the population rabbit immunoglobulins specifically binds Programmed Death-Ligand 1 (PD-L1).

In some embodiments, the composition is produced by immunizing a rabbit with an antigenic fragment of PD-L1.

In some embodiments, the antigenic fragment of PD-L1 is a PD-L1 extracellular domain.

In some embodiments, the antigenic fragment comprises, consists of, or consists essentially of amino acids 19-238 of a PD-L1 sequence according to SEQ ID NO: 15 or a variant thereof.

In some embodiments, the antigenic fragment shares at least 80%, 90%, 95%, 96%, 97%, 98%, 99%, or 100% identity to SEQ ID NO: 15 or a fragment thereof.

In some embodiments, the population of rabbit immunoglobulins binds MC38-hPD-L1 cells with at least as high affinity, or higher affinity than atezolizumab.

In some embodiments, the population of rabbit immunoglobulins binds to a non-small cell lung cancer cell, optionally one or more of H292, H460, H1975, HCC827, and H1299 cells.

In some embodiments, the population of rabbit immunoglobulins exhibit complement-dependent-cytotoxicity (CDC) activity, optionally at an IC₅₀ of about 0.72 μg/mL or less.

In some embodiments, the population of rabbit immunoglobulins inhibits tumor cell growth in vivo.

In some embodiments, the population of rabbit immunoglobulins exhibit antibody-dependent cellular toxicity (ADCC) activity.

In some embodiments, the population of rabbit immunoglobulins block PD-L1 from binding to the PD-1 receptor.

In some embodiments, the population of rabbit immunoglobulins blocks the PD-1 signaling pathway.

In some embodiments, the population of rabbit immunoglobulin enhances effector cell function, such as natural killer cells.

In some embodiments, the population of rabbit immunoglobulins has an avidity for PD-L1 of at least 0.1 1/sec, at least 0.01 1/sec, at least 0.001 1/sec at least 0.0001 1/sec, or at least 0.00001 I/sec.

In some embodiments, the population of rabbit immunoglobulins has an avidity for PD-L1 of 0.1 to 0.01 1/sec, 0.01 to 0.001 1/sec, or 0.0001 to 0.00001 1/sec.

In some embodiments, the population of rabbit immunoglobulin composition is substantially similar to ATCC Deposit No. PTA-127159 or wherein population of rabbit immunoglobulins has an avidity for PD-L1 at least 50%, at least 60%, at least 70%, at least 80%, at least 90%, at least 100%, at least 110%, or at least 120% that of ATCC Deposit No. PTA-127159.

In some embodiments, the disclosure provides a method of making polyclonal rabbit immunoglobulin specific for Programmed Death-Ligand 1 (PD-L1), comprising administering an antigenic fragment of PD-L1, or a polynucleotide encoding the antigenic fragment, to a rabbit, wherein the rabbit produces a population of rabbit immunoglobulins that specifically binds PD-L1.

In some embodiments, the method comprises administering the antigenic fragment or polynucleotide encoding the antigenic fragment 3, 4, 5, or more times.

In some embodiments, the method comprises collecting serum or plasma from the transgenic ungulate.

In some embodiments, the serum or plasma comprises a population of fully rabbit immunoglobulins.

In some embodiments, the antigenic fragment of PD-L1 is a PD-L1 extracellular domain.

In some embodiments, the antigenic fragment comprises, consists of, or consists essentially of amino acids 19-238 of a PD-L1 sequence according to SEQ ID NO: 15 or a variant thereof.

In some embodiments, the antigenic fragment shares at least 80%, 90%, 95%, 96%, 97%, 98%, 99%, or 100% identity to SEQ ID NO: 15 or a fragment thereof.

In some embodiments, the population of rabbit immunoglobulins binds MC38-hPD-L1 cells with at least as high affinity, or higher affinity than atezolizumab.

In some embodiments, the population of rabbit immunoglobulins binds to a non-small cell lung cancer cell, optionally one or more of H292, H460, H1975, HCC827, and H1299 cells.

In some embodiments, the population of rabbit immunoglobulins exhibit complement-dependent-cytotoxicity (CDC) activity, optionally at an IC₅₀ of about 0.72 μg/mL or less.

In some embodiments, the population of rabbit immunoglobulins inhibits tumor cell growth in vivo.

In some embodiments, the antigenic fragment is administering in a pharmaceutical composition comprising Montanide ISA-206 and/or Quil A.

In some embodiments, the method comprises a) administering a polynucleotide encoding the antigenic fragment of PD-L1; b) administering a polynucleotide encoding the antigenic fragment of PD-L1, three to four weeks later; c) administering the antigenic fragment of PD-L1, four weeks later d) administering the antigenic fragment of PD-L1, four weeks later; and e) administering the antigenic fragment of PD-L1, four weeks later.

In some embodiments, the method comprises purifying the rabbit immunoglobulin to produce a composition according to a method disclosed herein.

In some embodiments, the population of rabbit immunoglobulins exhibit antibody-dependent cellular toxicity (ADCC) activity.

In some embodiments, the population of rabbit immunoglobulins block PD-L1 from binding to PD-1.

In some embodiments, the population of rabbit immunoglobulins blocks the PD-1 signaling pathway.

In some embodiments, the population of rabbit immunoglobulin enhances effector cell function, optionally natural killer cells.

In some embodiments, the population of rabbit immunoglobulins has an avidity for PD-L1 of at least 0.1 1/sec, at least 0.01 1/sec, at least 0.001 1/sec at least 0.0001 1/sec, or at least 0.00001 1/sec.

In some embodiments, the population of rabbit immunoglobulins has an avidity for PD-L1 of 0.1 to 0.01 1/sec, 0.01 to 0.001 1/sec, 0.001 to 0.0001 1/sec, or 0.0001 to 0.00001 1/sec.

In some embodiments, the population of rabbit immunoglobulin composition is substantially similar to ATCC Deposit No. PTA-127159 or wherein population of rabbit immunoglobulins has an avidity for PD-L1 at least 50%, at least 60%, at least 70%, at least 80%, at least 90%, at least 100%, at least 110%, or at least 120% as great as that of ATCC Deposit No. PTA-127159.

In some embodiments, the composition optionally comprises one or more pharmaceutically acceptable excipients.

A method of treating or preventing cancer in a subject in need thereof, comprising administering an effective amount of a composition or pharmaceutical composition of the disclosure to the subject.

In another aspect, the disclosure provides a method of treating or preventing cancer in a subject in need thereof, comprising administering an effective amount of a composition of the disclosure or a pharmaceutical composition of the disclosure to the subject.

Additional embodiments, features, and advantages of the invention will be apparent from the following detailed description and through practice of the invention.

### BRIEF DESCRIPTION OF THE DRAWINGS

FIGS. 1A-1H show construction of the isHAC and isKcHACΔ vectors.

FIG. 1A shows a flow of the isHAC and isKcHACΔ vector construction. The bovinizing vector, pCC1BAC-isHAC, is BAC-based (backbone is pCC1BAC vector), consisting of 10.5 kb and 2 kb of genomic DNA as a long and short arm, respectively, 9.7 kb of the bovine genomic DNA covering the bovine Iγ1-Sγ1 and its surrounding region to replace the human corresponding 6.8 kb of Iγ1-Sγ1 region, the chicken β-actin promoter-driven neo gene flanked by FRT sequence and DT-A gene. After the targeted bovinization, the neo cassette is removed by FLP introduction.

FIG. 1B shows detailed information on the targeting vector pCC1BAC-isHAC. The 2 kb of Afe I-Bam HI fragment and 10.5 kb of Apa I-Hpa I fragment for a short arm and long arm were obtained from clone h10 and clone h18/h20, respectively, derived from λ, phage genomic library constructed from CHO cells containing the κHAC by screening using a probe around the human Iγ1-Sγ1 region. The 9.7 kb fragment (5′ end through Bsu36 I) was obtained from clone b42 derived from the λ phage bovine genomic library.

FIG. 1C shows genotyping of the bovinized Iγ1-Sγ1 region. Five sets of genomic PCR were implemented, as indicated. iscont1-F1/R1 is a positive PCR specific to the homologous recombination. iscont1-F1×hIgG1-R10 is a negative PCR that is prohibited by the presence of the neo cassette. isHAC-Sw-dig-F5/R3 and isHAC-TM-dig-F3/R2 are for structural integrity check of their corresponding region, digested by Bam HI+Pvu II and Age I, Sma I or Pvu II, respectively. bNeo 5′-R×bIgG1-5′-seq-R6 is to confirm the presence of FRT sequence.

FIG. 1D shows genotyping after the FLP-FRT deletion of the neo cassette.

FIG. 1E shows extensive genomic PCR for genotyping of the isHAC vector. Location of

each genomic PCR primer pair is depicted in relation to the isHAC vector structure.

FIG. 1F shows CGH analysis among three different CHO clones containing the isHAC vector. DNA from is C1-133 was used as a reference. There was no apparent structural difference of the isHAC among the three cell lines.

FIG. 1G shows extensive genomic PCR for genotyping of the isKcHACΔ vector. Location of each genomic PCR primer pair is depicted in relation to the isKcHACΔ vector structure.

FIG. 1H shows CGH analysis among three different CHO clones containing the isKcHACΔ vector. DNA from isKCDC15-8 was used as a reference. There was no apparent structural difference of the isKcHACΔ among the three cell lines.

FIG. 2 shows SDS-PAGE (left) and western immunoblot (right) analysis of the indicated recombinant PD-L1 proteins.

FIG. 3 shows binding of rabbit anti-PD-L1 polyclonal antibody (pAb) (top) and atezolizumab monoclonal antibody (bottom) to cells expressing PD-L1 by flow cytometry. MDA-MB-231 expresses high levels of PD-L1, COL0205 expresses low PD-L1, and the genetically modified mouse cell line, MC38-hPD-L1, expresses high levels of human PD-L1. MC38 does not express the human PD-L1 gene, and it was used as a negative control.

FIG. 4 shows complement mediated cytotoxicity (CDC) of atezolizumab monoclonal antibody (bottom), which is a humanized IgG1 FcR-deficient monoclonal antibody, or rabbit anti-PD-L1 polyclonal antibody (pAb) (top).

FIG. 5 shows mean tumor weights (left) and percentage of mouse survival (right) over time for groups treated with saline, anti-PD-L1 pAb, atezolizumab (Tecentriq™), or pembrolizumab (Keytruda™). All agents were dosed at 0.1 mg.

FIG. 6 shows mean tumor weights (left) and percentage of mouse survival (right) over time for groups treated with anti-PD-L1 pAb at 0.05 mg, 0.025 mg, 0.0125 mg, or 0.003125 mg; or with atezolizumab (Tecentriq™). Dosing was performed on days 12, 14, 18, and 21.

FIG. 7 shows serum titers of SAB-162P human anti-PD-L1 specific antibodies. The titer increases with each subsequent Tc bovine vaccination. An indirect ELISA demonstrates anti-PD-L1 specific titers (y-axis) with corresponding vaccination number and serum harvest day (x-axis).

FIG. 8A-8B show that SAB-162P binds recombinant and cellular PD-L1 protein.

FIG. 8A shows indirect ELISA analysis showing the titer of purified SAB-162P compared to negative control (NC) human IgG purified from Tc bovine pre-immune plasma. Data represent technical replicates. Error bars are SD.

FIG. 8B shows flow cytometry analysis of SAB-162P binding to PD-L1 positive NSCLC cells lines (H292, H460, H1975, and HCC827). NSCLC cell line H1299 has low levels of PD-L1, and Raji and Ramos lymphoma cells are PD-L1 negative. Bars represent mean fluorescence intensity (MFI) of ≥3,000 singlet live cells on the y-axis. Cell lines are indicated on the x-axis. Representative data from one biological replicate of duplicate experiments is shown.

FIG. 9A-9C show human NSCLC cellular binding titrations of SAB-162P. SAB-162P (closed circles) binding levels were compared to the saturating MFI level of atezolizumab (dotted line). The maximum fluorescence intensity (MFI) level is on the y-axis. Each serial antibody dilution represents ≥3,000 singlet live cells for each data point. Representative data from one biological replicate of duplicate experiments is shown.

FIG. 9A shows the titration of SAB-162P binding to the NSCLC cell line, HCC827.

FIG. 9B shows the titration of SAB-162P binding to the NSCLC cell line, H292.

FIG. 9C shows the titration of SAB-162P binding to the NSCLC cell line, H1975.

FIG. 10A-10D show SAB-162P induces ADCC activation of engineered Jurkat Lucia NFAT CD16 cells after 24 hours. Activation of effector cells is depicted by luciferase activity in RLU on the y-axis verses increasing SAB-162P (closed circles) antibody concentration depicted on the x-axis. SAB naïve negative control (NC) IgG is shown only at the highest antibody concentration (open circle). Data points are means of technical triplicates. Error bars indicate SD. Representative data from one replicate of biological duplicate experiments are shown.

FIG. 10A shows ADCC activity against target NSCLC cell line, HCC827 (6:1 E:T cell ratio).

FIG. 10B shows ADCC activity against the target NSCLC cell line, H460 (6:1 E:T cell ratio).

FIG. 10C shows ADCC activity against the PD-L1 expressing NSCLC cell line, H1975 (6:1 E:T cell ratio).

FIG. 10D shows ADCC activity against target NSCLC cell line, H292 (6:1 E:T cell ratio).

FIG. 11A-11B show that SAB-162P blocks PD-1 binding to PD-L1 expressing human NSCLC cell lines. Cells were pre-incubated with increasing amounts of SAB-162P. Saturating levels of PE-labeled human PD-1-Fc-His fusion protein were added, and the mean fluorescence intensity (MFI) was measured by flow cytometry. SAB-162P blockage of PD-1 binding is indicated by closed circles. The negative control (NC) IgG is shown only at the highest concentration (open circle). Maximum binding of PD-1-Fc-His without antibody is indicated by an open triangle. Each data point is MFI from ≥3,000 singlet live cells. Representative data from one replicate of biological duplicate experiments is shown for each human NSCLC cell line.

FIG. 11A shows mean fluorescence intensity of PE-labeled human PD-1-Fc-His fusion protein binding to NSCLC cells line, HCC827 preincubated with increasing concentrations of SAB-162P.

FIG. 11B shows mean fluorescence intensity of PE-labeled human PD-1-Fc-His fusion protein binding to NSCLC cells line, H1975, preincubated with increasing concentrations of SAB-162P.

FIG. 12 shows SAB-162P binding to PD-L1 blocks PD-1 T cell inhibitory signaling. Loss of NFAT responsive luciferase reporter inhibition from Jurkat human PD-1 expressing T cells engaged with human PD-L1 aAPC/CHO-K1 cells is shown as RLU on the y axis. Increasing concentrations of SAB-162P (closed circles) or SAB NC IgG (open circles) were preincubated with the aAPC/CHO-K1 cells prior to the addition of Jurkat T cells. Increased luciferase expression represents release of PD-1 mediated T cell inhibition. Data points are means of technical triplicates. Error bars, SD. One representative data set from biological duplicate experiments is shown.

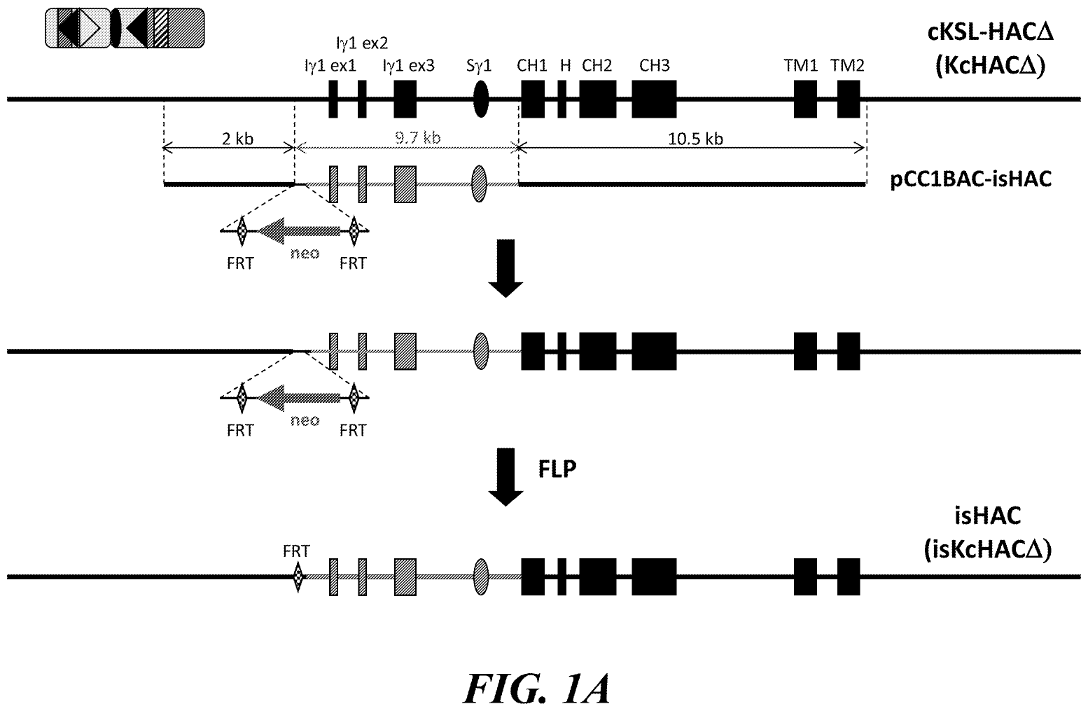

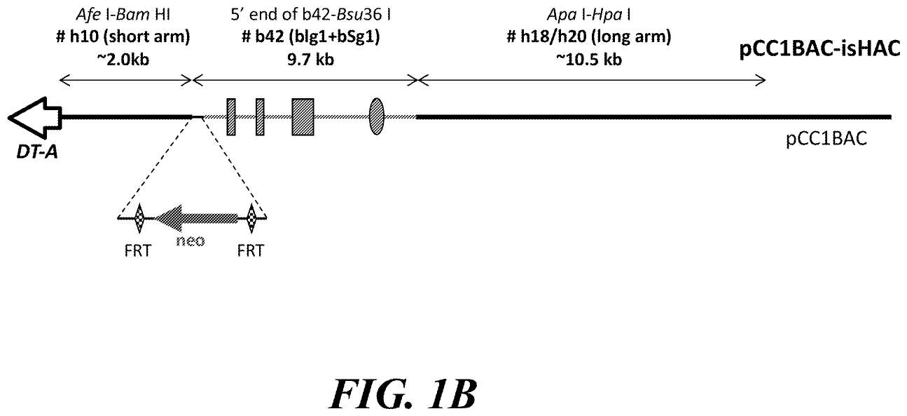

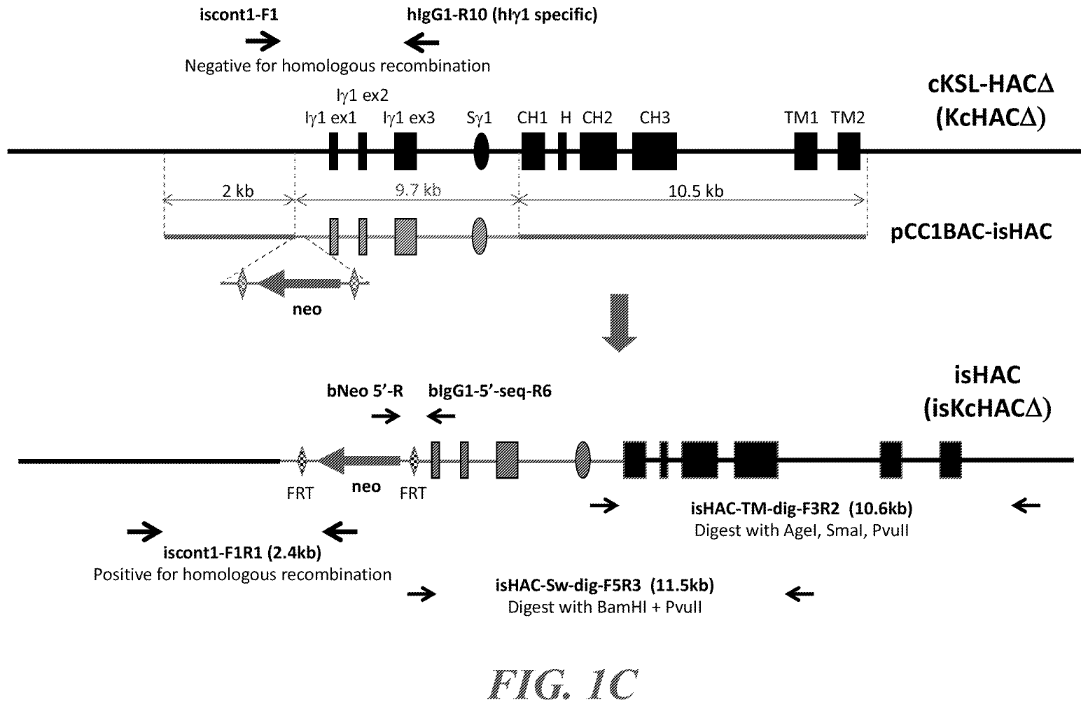

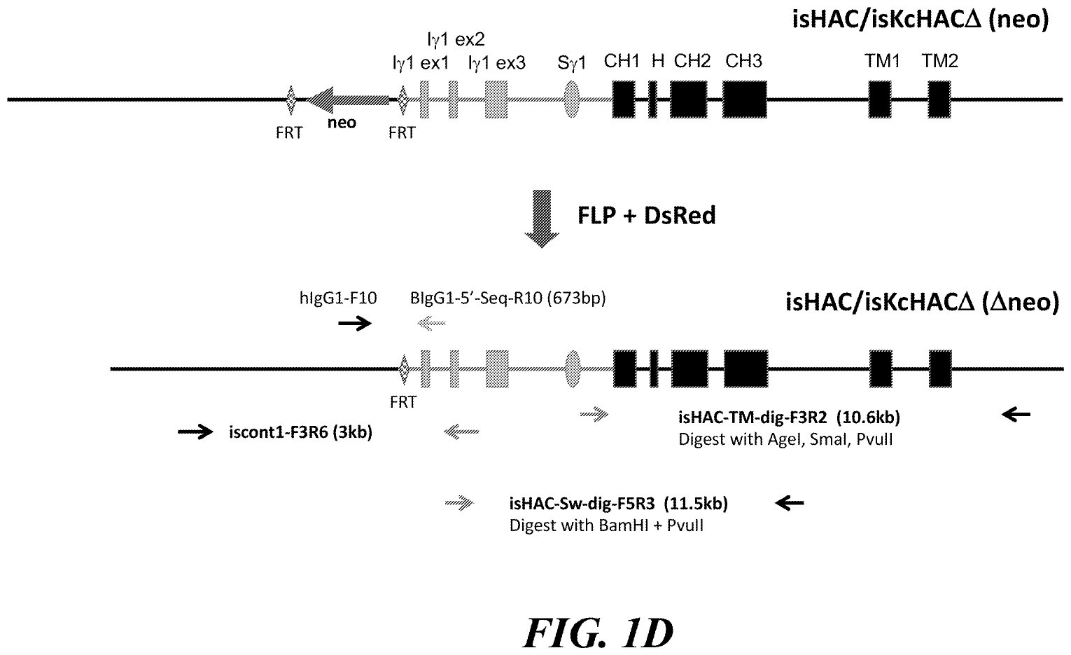

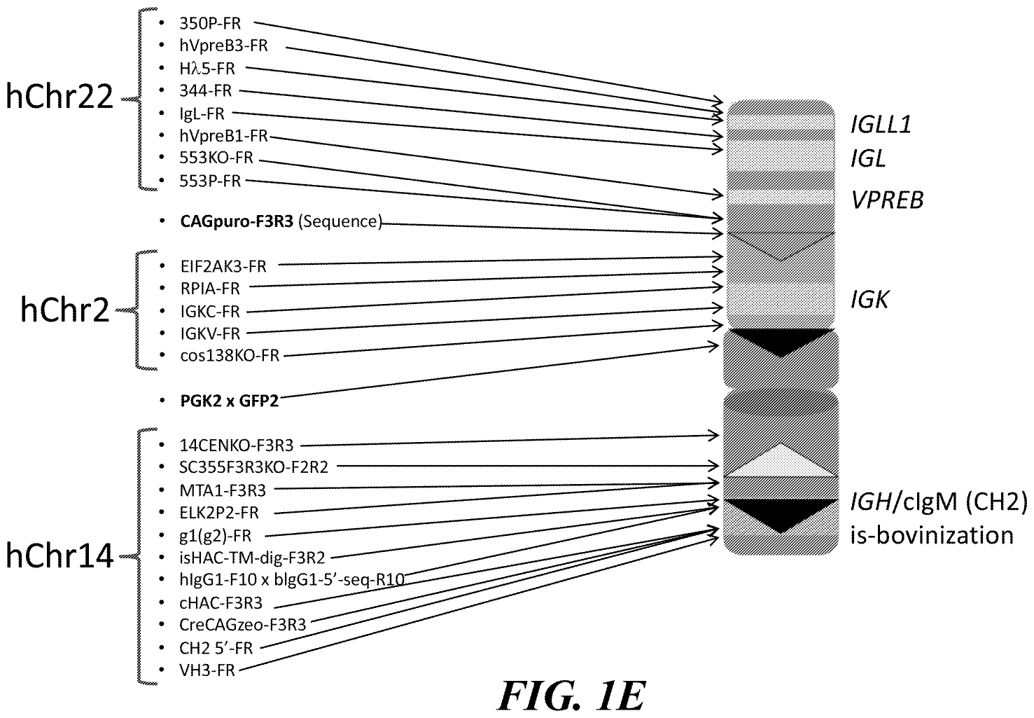

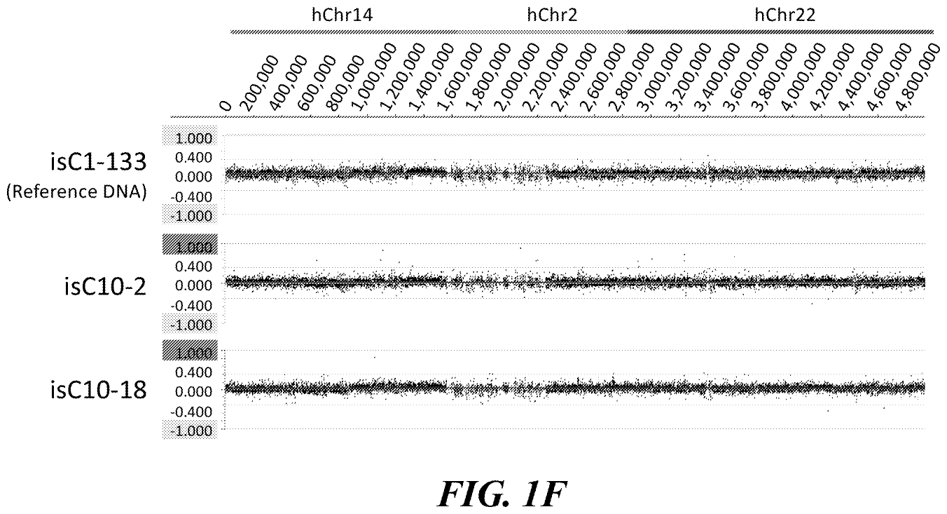

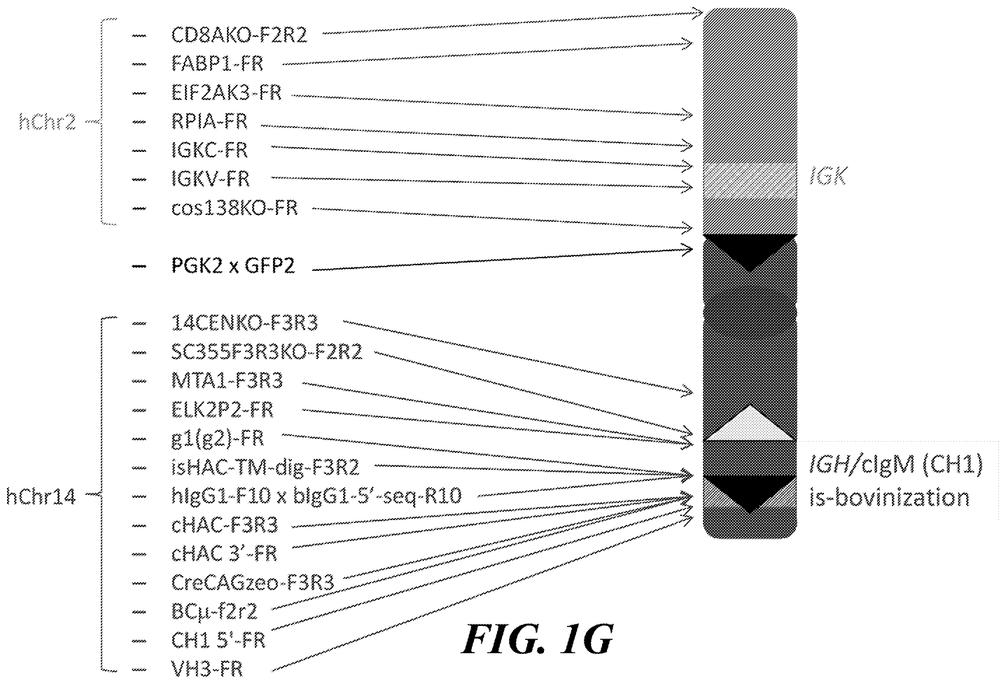

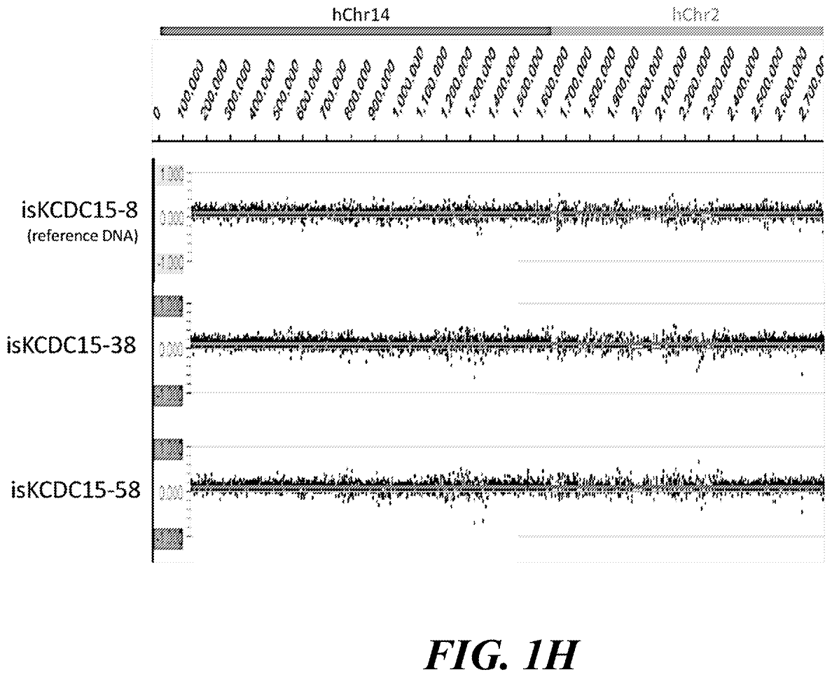

### DETAILED DESCRIPTION

The present inventors have developed a human immunoglobulin product for human disease that overcomes limitations of monoclonal antibody therapy. Transgenic animals with the endogenous Ig locus replaced by a human artificial chromosome encoding a human Ig locus express fully human polyclonal antibodies. Immunization of such a transgenic animal with a recombinant PD-L1 protein, or an antigenic fragment thereof, and/or with a polynucleotide encoding the antigen, generates polyclonal immunoglobulin with yield, purity, and antigen specificity that enable use of this product in medical applications. Various embodiments of the invention are provided in the description that follows.

### Definitions

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a” “an” and “the” include plural referents unless the context clearly dictates otherwise. “And” as used herein is interchangeably used with “or” unless expressly stated otherwise.

All embodiments of any aspect of the invention can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” “below,” and words of similar import, when used in this application, shall refer to this application as a whole and not to any particular portions of the application.

The term “ungulate” refers to any suitable ungulate, including but not limited to bovine, pig, horse, donkey, zebra, deer, oxen, goats, sheep, and antelope.

The term “transgenic” means the cells of the ungulate comprise one or more polynucleotides encoding exogenous gene(s) (e.g. an immunoglobulin locus). Such as polynucleotide may be a portion of an artificial chromosome. Alternatively, or in addition to an artificial chromosome, one or more polynucleotides encoding exogenous gene(s) may be integrated into the genome of the cells of the ungulate.

The terms “polyclonal” or “polyclonal serum” or “polyclonal plasma” or “polyclonal immunoglobulin” refer to a population of immunoglobulins having shared constant regions but diverse variable regions. The term polyclonal does not, however, exclude immunoglobulins derived from a single B cell precursor or single recombination event, as may be the case when a dominant immune response is generated. A polyclonal serum or plasma contains soluble forms (e.g., IgG) of the population of immunoglobulins. The term “purified polyclonal immunoglobulin” refers to polyclonal immunoglobulin purified by serum or plasma. Methods of purifying polyclonal immunoglobulin include, without limitation, caprylic acid fractionation and adsorption with red blood cells (RBCs).

A “population” of immunoglobulins refers to immunoglobulins having diverse sequences, as opposed to a sample having multiple copies of a single immunoglobulin. Similarly stated, the term population excludes immunoglobulins secreted from a single B cell, plasma cell, or hybridoma in culture, or from a host cells transduced or transformed with recombinant polynucleotide(s) encoding a single pair of heavy and light chain immunoglobulin sequences.

The term “immunoglobulin” refers to a protein complex of at least two heavy and at least two light chains in 1:1 ratio, including any of the five classes of immunoglobulin—IgM, IgG, IgA, IgD, IgE. In variations, the immunoglobulin is engineered in any of various ways known in the art or prospectively discovered, including, without limitation, mutations to change glycosylation patterns and/or to increase or decrease complement dependent cytotoxicity.

An immunoglobulin is “fully human or substantially human” when the protein sequence of the immunoglobulin is sufficiently similar to the sequence of a native human immunoglobulin that, when administered to a subject, the immunoglobulin generates an anti-immunoglobulin immune response similar to, or not significantly worse, that the immune reaction to native human immunoglobulin. A fully human immunoglobulin will comprise one or more substitutions, insertions, to deletions in variable regions, consistent with recombination, selection, and affinity maturation of the immunoglobulin sequence. In variations, the fully human or substantially human immunoglobulin is engineered in any of various ways known in the art or prospectively discovered, including, without limitation, mutations to change glycosylation patterns and/or to increase or decrease complement dependent cytotoxicity.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. Any of the compositions of the present disclosure may be isolated compositions.

The percentage of an immunoglobulin (e.g., immunoglobulin that specifically binds PD-L1) “by mass of total immunoglobulin” refers to the concentration of a target immunoglobulin population divided by the concentration of total immunoglobulin in a sample, multiplied by 100. The concentration of target immunoglobulin can be determined by, for example, affinity purification of target immunoglobulin (e.g. on affinity column comprising PD-L1) followed by concentration determination.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation. Alternatively, “about” can mean plus or minus a range of up to 20%, up to 10%, or up to 5%.

The terms “immunization” and “immunizing” refer to administering a composition to a subject (e.g., a transgenic ungulate) in an amount sufficient to elicit, after one or more administering steps, a desired immune response (e.g., a polyclonal immunoglobulin response specific to PD-L1). Administration may be by intramuscular injection, intravenous injection, intraperitoneal injection, or any other suitable route. Immunization may comprise between one and ten, or more administrations (e.g. injections) of the composition, such as 1, 2, 3, 4, 5, 6, 7, 8, 9, 10, or more administrations. The first administration may elicit no detectable immune response as generally each subsequence administration will boost the immune response generated by prior administrations.

The term “target antigen” refers to any antigen use to elicit a desired immune response. The target antigen used to generate an immunoglobulin product may be recombinant PD-L1 or an antigenic fragment thereof, or nucleic acid that encodes such proteins (e.g. RNA, linear DNA, or plasmid DNA).

The term “purify” refers to separating a target cell or molecule (e.g. a population of immunoglobulins) from other substances present in a composition. Immunoglobulins may be purified by fractionation of plasma, by affinity (e.g. protein A or protein G binding, or other capture molecule), by charge (e.g. ion-exchange chromatography), by size (e.g. size exclusion chromatograph), or otherwise. Purifying a population of immunoglobulins may comprise treating a composition comprising the population of immunoglobulins with one or more of acids, bases, salts, enzymes, heat, cold, coagulation factors, or other suitable agents. Purifying may further include adsorption of a composition comprising a target cell or molecule and an impurity onto non-target cells or molecules (e.g., red blood cells) to partially or completely remove the impurity. Purifying may further include pre-treatment of serum or plasma, e.g., caprylic acid fractionation.

The terms “treating” and “treatment” refer to one or more of relieving, alleviating, delaying, reducing, reversing, improving, or managing at least one symptom of a condition in a subject. The term “treating” may also mean one or more of arresting, delaying the onset (i.e., the period prior to clinical manifestation of the condition) or reducing the risk of developing or worsening a condition.

The term “pharmaceutically acceptable” means biologically or pharmacologically compatible for in vivo use in animals or humans, and can mean approved by a regulatory agency of the Federal or a state government or listed in the U.S. Pharmacopeia or other generally recognized pharmacopeia for use in animals, and more particularly in humans.

The term “hyperimmunized” refers to immunization regimen that generates an immune response to the subject greater than required to produce a desired titer (e.g. a binding titer) after dilution of the immunoglobulin produced by the subject. For example, if a desired titer is 1:100, one may hyperimmunize an animal by a prime immunization followed by one, two, three or more boost immunizations to produce a 1:1,000 titer, or greater titer, in the subject-so that immunoglobulin produced by the subject may be diluted in the production of a biotherapeutic in order to give a desired titer in the biotherapeutic.

The term “affinity” refers to the strength of the interaction between an epitope and an antibody's antigen binding site. The affinity can be determined, for example, using the equation

KA=[Ab-Ag]/[Ab][Ag]

Where KA=affinity constant; [Ab]=molar concentration of unoccupied binding sites on the antibody; [Ag]=molar concentration of unoccupied binding sites on the antigen; and [Ab-Ag]=molar concentration of the antibody-antigen complex. The K_{A} describes how much antibody-antigen complex exists at the point when equilibrium is reached. The time taken for this to occur depends on rate of diffusion and is similar for every antibody. However, high-affinity antibodies will bind a greater amount of antigen in a shorter period of time than low-affinity antibodies. The K_{A} of the antibodies produced can vary and range from between about 10⁻⁵ mol⁻¹ to about 10¹² mol⁻¹ or more. The K_{A} can be influenced by factors including pH, temperature, and buffer composition.

The antibody affinity can be measured using any means commonly employed in the art, including but not limited to the use of biosensors, such as surface plasmon resonance (SPR). Resonance units are proportional to the degree of binding of soluble ligand to the immobilized receptor (or soluble antibody to immobilized antigen). Determining the amount of binding at equilibrium with different known concentrations of receptor (antibody) and ligand (protein antigen) allows the calculation of equilibrium constants (K_{A}, K_{D}), and the rates of dissociation and association (k_{off}, kₒₙ).

The term “avidity” refers to the accumulated strength of multiple affinities of individual non-covalent binding interactions, such as between an antibody and its antigen. Avidity is related to both the affinity of individual immunoglobulin molecules in the population with specific epitopes, and also the valencies of the immunoglobulins and the antigen. For example, the interaction between a bivalent monoclonal antibody and an antigen with a highly repeating epitope structure, such as a polymer, would be one of high avidity. Avidity is measured by the off rate (k_{off}).

For example, KD (the equilibrium dissociation constant) is a ratio of k_{off}/kₒₙ, between the antibody and its antigen. KD and affinity are inversely related. The lower the KD value (lower antibody concentration), the higher the affinity of the antibody. Most antibodies have KD values in the low micromolar (10⁻⁶) to nanomolar (10⁻⁷ to 10⁻⁹) range. High affinity antibodies are generally considered to be in the low nanomolar range (10⁻⁹) with very high affinity antibodies being in the picomolar (10⁻¹²) range or lower (e.g. 10¹³ to 10⁻¹⁴ range). In one embodiment, the antibodies produced by immunization with the PD-L1-hFc antigen disclosed herein have a KD ranging from about 10⁻⁶ to about 10⁻¹⁵, from about 10⁻⁷ to about 10⁻¹⁵, from about 10⁻⁸ to about 10⁻¹⁵, and from about 10⁻⁹ to about 10⁻¹⁵, from about 10⁻¹⁰ to about 10⁻¹⁵, about 10⁻¹¹ to about 10⁻¹⁵, about 10⁻¹² to about 10⁻¹⁵, about 10¹³ to about 10⁻¹⁴, about 10⁻¹³ to about 10⁻¹⁵, and about 10⁻¹⁴ to about 10⁻¹⁵.

The population of human immunoglobulins produced by the methods disclosed herein have high avidity, indicating they bind tightly to the antigen. In one embodiment, the antibodies produced by immunization with the PD-L1-hFc antigen disclosed herein have an avidity ranging from about 10⁻¹ 1/sec to about 10⁻¹³ 1/sec, from about 10⁻³ 1/sec to about 10⁻¹³ 1/sec, from about 10⁻⁵ 1/sec to about 10⁻¹³1/sec, from about 10⁻⁶¹/sec to about 10⁻¹³1/sec, from about 10⁻⁷¹/sec to about 10⁻¹⁵ 1/sec, from about 10⁻⁸ 1/sec to about 10⁻¹⁵ 1/sec, from about 10⁻⁹ 1/sec to about 10⁻¹⁵ 1/sec, from about 10⁻¹⁰ 1/sec to about 10⁻¹³ 1/sec, from about 10⁻¹¹ 1/sec to about 10⁻¹³ 1/sec, or from about 10⁻¹² 1/sec to about 10⁻¹³ 1/sec.

An immunoglobulin is “specific to” or “specifically binds” (used interchangeably herein) to a target (e.g., PD-L1) is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An immunoglobulin “specifically binds” to a particular protein or substance if it binds with greater affinity, avidity, more readily, and/or with greater duration than it binds to alternative particular protein or substance. For example, an immunoglobulin that specifically or preferentially binds to PD-L1 is an immunoglobulin that binds PD-L1 with greater affinity, avidity, more readily, and/or with greater duration than it binds to other proteins. An immunoglobulin that specifically binds to a first protein or substance may or may not specifically or preferentially bind to a protein (e.g., a member of the B7 family of immune-regulatory ligands), cell, or substance. As such, “specific binding” does not necessarily require (although it can include) exclusive binding. Generally, but not necessarily, reference to binding means specific binding.

The term “HAC vector” means a vector which comprises at least a human chromosome-derived centromere sequence, a telomere sequence, and a replication origin, and may contain any other sequences as desired for a given application. When present in a host cell, the HAC vector exists independently from a host cell chromosome in the nucleus. Any suitable methods can be used to prepare HAC vectors and to insert nucleic acids of interest into the HAC, including but not limited to those described in the examples that follow. The HAC vector is a double stranded DNA vector, as is known to those of skill in the art.

### EMBODIMENTS

Provided are methods of making a human polyclonal immunoglobulin for treatment of cancer, comprising administering an antigen comprising a PD-L1 or antigenic fragment thereof, or a polynucleotide encoding the antigen, to a transgenic ungulate, wherein the transgenic ungulate comprises a genome comprising a human immunoglobulin locus or an artificial chromosome comprising a human immunoglobulin locus, wherein the transgenic ungulate produces a population of human immunoglobulins that specifically binds the PD-L1.

In a variation, non-human PD-L1, or a polynucleotide encoding it, is used (e.g., a domesticated animal such as a dog, cat, sheep, etc.). The transgenic ungulate may in such cases comprise an artificial chromosome encoding an Ig locus of the non-human species such that antibodies of that species are generated.

In some embodiments, the PD-L1, or a polynucleotide encoding it (that is, “the antigen”) is administered before, during, or after administration of one or more adjuvants. In some embodiments, the antigen and one or more adjuvants are administered together in a single composition, comprising optionally one or more pharmaceutically acceptable excipients.

Illustrative adjuvants include an aluminum salt adjuvant, an oil in water emulsion (e.g. an oil-in-water emulsion comprising squalene, such as MF59 or AS03), a TLR7 agonist (such as imidazoquinoline or imiquimod), or a combination thereof. Suitable aluminum salts include hydroxides (e.g. oxyhydroxides), phosphates (e.g. hydroxyphosphates, orthophosphates), (e.g. see chapters 8 & 9 of Vaccine Design. (1995) eds. Powell & Newman. ISBN: 030644867X. Plenum), or mixtures thereof. Further illustrative adjuvants include, but are not limited to, Adju-Phos, Adjumerlm, albumin-heparin microparticles, Algal Glucan, Algammulin, Alum, Antigen Formulation, AS-2 adjuvant, autologous dendritic cells, autologous PBMC, Avridine™, B7-2, BAK, BAY R1005, Bupivacaine, Bupivacaine-HCl, BWZL, Calcitriol, Calcium Phosphate Gel, CCR5 peptides, CFA, Cholera holotoxin (CT) and Cholera toxin B subunit (CTB), Cholera toxin Al-subunit-Protein A D-fragment fusion protein, CpG, CRL1005, Cytokine-containing Liposomes, D-Murapalmitine, DDA, DHEA, Diphtheria toxoid, DL-PGL, DMPC, DMPG, DOC/Alum Complex, Fowlpox, Freund's Complete Adjuvant, Gamma Inulin, Gerbu Adjuvant, GM-CSF, GMDP, hGM-CSF, hIL-12 (N222L), hTNF-alpha, IFA, IFN-gamma in pcDNA3, IL-12 DNA, IL-12 plasmid, IL-12/GMCSF plasmid (Sykes), IL-2 in pcDNA3, IL-2/Ig plasmid, IL-2/Ig protein, IL-4, IL-4 in pcDNA3, Imiquimod, ImmTher™, Immunoliposomes Containing Antibodies to Costimulatory Molecules, Interferon-gamma, Interleukin-1 beta, Interleukin-12, Interleukin-2, Interleukin-7, ISCOM(s)™, Iscoprep 7.0.3™, MONTANIDE™ ISA-25, Keyhole Limpet Hemocyanin, Lipid-based Adjuvant, Liposomes, Loxoribine, LT(R192G), LT-OA or LT Oral Adjuvant, LT-R192G, LTK63, LTK72, MF59, MONTANIDE ISA 51, MONTANIDE ISA 720, MPL.TM., MPL-SE, MTP-PE, MTP-PE Liposomes, Murametide, Murapalmitine, NAGO, nCT native Cholera Toxin, Non-Ionic Surfactant Vesicles, non-toxic mutant E1 12K of Cholera Toxin mCT-E112K, p-Hydroxybenzoique acid methyl ester, pCIL-10, pCIL12, pCMVmCAT1, pCMVN, Peptomer-NP, Pleuran, PLG, PLGA, PGA, and PLA, Pluronic L121, PMMA, PODDS™, Poly rA: Poly rU, Polysorbate 80, Protein Cochleates, QS-21, Quadri A saponin, Quil-A, ISA-25/Quil-A, Rehydragel HPA, Rehydragel LV, RIBI, Ribilike adjuvant system (MPL, TMD, CWS), S-28463, SAB-adj-1, SAB-adj-2, SAF-1, Sclavo peptide, Sendai Proteoliposomes, Sendai-containing Lipid Matrices, Span 85, Specol, Squalane 1, Squalene 2, Stearyl Tyrosine, Tetanus toxoid (TT), Theramide™, Threonyl muramyl dipeptide (TMDP), Ty Particles, and Walter Reed Liposomes.

The immunization may be carried out by administering the antigen with, for example, a complete Freund's adjuvant or an appropriate adjuvant such as an aluminum hydroxide gel, and pertussis bacteria vaccine, subcutaneously, intravenously, or intraperitoneally into a transgenic ungulate. In one embodiment, the immunization comprises hyperimmunization. In various embodiments, the antigen is administered once to 10 times every 1 to 4 weeks after the first administration. After 1 to 14 days from each administration, blood is collected from the animal to measure the antibody value of the serum.

In some embodiments, the antigen is administered 3, 4, 5, 6 or more times. Administration of the PD-L1 may be performed, e.g., every 1-2 weeks, 2-3 weeks, 3-4 weeks, 4-5 weeks, 5-6 weeks, or 6-7 weeks, or longer intervals, e.g., every 1 week, 2 weeks, 3 weeks, 4 weeks, 5 weeks or 6 weeks. After each immunization, serum and/or plasma may be harvested from the transgenic ungulate one or more times. For example, the method may be including performing controls bleeds two or three times at intervals about 7-14 days.

In embodiments of the methods of the disclosure, the genome of the transgenic ungulate comprises a human immunoglobulin locus. Illustrative methods are provided in U.S. Pat. Nos. 9,902,970; 9,315,824; 7,652,192; and 7,429,690; and 7,253,334, the disclosure of which are incorporated by reference herein for all purposes. Further illustrative methods are provided by Kuroiwa, Y., et al. (2009) Nat Biotechnol. 27(2):173-81, and Matsushita et al. (2015) PLoS ONE 10(6):e0130699.

The disclosure provides a human artificial chromosome (HAC) vector comprising genes encoding:
(a) one or more human antibody heavy chains, wherein each gene encoding an antibody heavy chain is operatively linked to a class switch regulatory element;
(b) one or more human antibody light chains; and
(c) one or more human antibody surrogate light chains, and/or an ungulate-derived IgM heavy chain constant region;
wherein at least one class switch regulatory element of the genes encoding the one or more human antibody heavy chains is replaced with an ungulate-derived class switch regulatory element.

The HAC vectors of the disclosure can be used, for example, for large-scale production of fully human antibodies by transgenic animals, as described for the methods of the invention. The HAC vector of the present disclosure comprises one or more genes encoding a human antibody heavy chain. Any human antibody heavy chain or combinations of human antibody heavy chains in combination may be encoded by one or more nucleic acids on the HAC. In various embodiments, 1, 2, 3, 4, 5, 6, 7, 8, or all 9 of human antibody heavy chains IgM, IgG1, IgG2, IgG3, IgG4, IgA1, IgA2, IgE and IgD may be encoded on the HAC in one or more copies. In one embodiment, the HAC comprises a human IgM antibody heavy chain encoding gene, alone or in combinations with 1, 2, 3, 4, 5, 6, 7, or the other 8 human antibody chain encoding genes. In one preferred embodiment, the HAC comprises a gene encoding at least a human IgG1 antibody heavy chain; in this embodiment, it is further preferred that the HAC comprises a gene encoding a human IgM antibody heavy chain or a gene encoding a human IgM antibody heavy chain that has been chimerized to encode an ungulate-derived IgM heavy chain constant region (such as a bovine heavy chain constant region). In another embodiment, the HAC comprises a gene encoding at least a human IgA antibody heavy chain; in this embodiment, it is further preferred that the HAC comprises a gene encoding a human IgM antibody heavy chain or a gene encoding a human IgM antibody heavy chain that has been chimerized to encode an ungulate-derived IgM heavy chain constant region (such as a bovine heavy chain constant region). In another preferred embodiment, the HAC comprises genes encoding all 9 antibody heavy chains, and more preferably where the gene encoding a human IgM antibody heavy chain has been chimerized to encode an ungulate-derived IgM heavy chain constant region. In another embodiment, the HAC may comprise a portion of human chromosome 14 that encodes the human antibody heavy chains. The variable region genes and the constant region genes of the human antibody heavy chain form a cluster and the human heavy chain locus is positioned at 14q32 on human chromosome 14. In one embodiment, the region of human chromosome 14 inserted in the HAC comprises the variable region and the constant region of the human antibody heavy chains from the 14q32 region of human chromosome 14.

In some embodiments of the HAC vectors of the present disclosure, at least one class switch regulatory element of the human antibody heavy chain encoding nucleic acid is replaced with an ungulate-derived class switch regulatory element. The class switch regulatory element refers to nucleic acid which is 5′ to an antibody heavy chain constant region. Each heavy chain constant region gene is operatively linked with (i.e. under control of) its own switch region, which is also associated with its own I-exons. Class switch regulatory elements regulate class switch recombination and determine Ig heavy chain isotype. Germline transcription of each heavy chain isotype is driven by the promoter/enhancer elements located just 5′ of the I-exons and those elements are cytokine or other activator-responsive. In a simple model of class switch, the specific activators and/or cytokines induce each heavy chain isotype germline transcription from its class switch regulatory element (i.e., activator/cytokine-responsive promoter and/or enhancer). Class switch is preceded by transcription of I-exons from each Ig heavy (IGH) locus-associated switch region. As each heavy chain constant region gene is linked with its own switch region.

Any suitable ungulate-derived class switch regulatory element can be used. For example, the human heavy chain gene isotypes listed below has the following class switch regulatory elements:
IgM: Iμ-Sμ
IgG1: Iγ1-Sγ1,
IgG2: Iγ2-Sγ2,
IgG3: Iγ3-Sγ3,
IgG4: Iγ4-Sγ4,
IgA1: Iα1-Sα1,
IgA2: Iα2-Sα2, and
IgE: Iε-Sε.

In various embodiments, 1, more than 1, or all of the human antibody heavy chain genes on the HAC have their class switch regulatory element replaced with an ungulate-derived class switch regulatory element, including but not limited to ungulate Iμ-Sμ, Iγ-Sγ, Iα-Sα, or Iε-Sε, class switch regulatory elements. In one embodiment, an Iγ1-Sγ1 human class switch regulatory element for human IgG1 heavy chain encoding nucleic acid on the HAC (such as that in SEQ ID NO: 1) is replaced with an ungulate Iγ1-Sγ1 class switch regulatory element. Exemplary ungulate Iγ1-Sγ1 class regulatory switch elements include a bovine IgG1 Iγ1-Sγ1 class switch regulatory element (SEQ ID NO: 2), a horse Iγ1-Sγ1 class switch regulatory element (SEQ ID NO: 3), and a pig Iγ1-Sγ1 class switch regulatory element (SEQ ID: 4). However, it is not necessary to replace the human class switch regulatory element with an ungulate class switch regulatory element from the corresponding heavy chain isotype. Thus, for example, an Iγ3-Sγ3 human class switch regulatory element for human IgG3 heavy chain encoding nucleic acid on the HAC can be replaced with an ungulate Iγ1-Sγ1 class switch regulatory element. As will be apparent to those of skill in the art based on the teachings herein, any such combination can be used in the HACs of the disclosure.

In another embodiment, the HAC comprises at least one ungulate enhancer element to replace an enhancer element associated with one or more human antibody heavy chain constant region encoding nucleic acids on the HAC. There are two 3′ enhancer regions (Alpha 1 and Alpha 2) associated with human antibody heavy chain genes. Enhancer elements are 3′ to the heavy chain constant region and also help regulate class switch. Any suitable ungulate enhancer can be used, including but not limited to 3′Eα enhancers. Non-limiting examples of 3′ Eα enhancers that can be used include 3′Eα, 3′Eα1, and 3′Eα2. Exemplary 3′Eα enhancer elements from bovine that can be used in the HACs and replace the human enhancer include, but are not limited to bovine HS3 enhancer (SEQ ID NO: 5), bovine HS12 enhancer (SEQ ID NO: 6), and bovine enhancer HS4. This embodiment is particularly preferred in embodiments wherein the HAC comprises the variable region and the constant region of the human antibody heavy chains from the 14q32 region of human chromosome 14.

The HAC vectors of the present disclosure may comprise one or more genes encoding a human antibody light chain. Any suitable human antibody light chain-encoding genes can be used in the HAC vectors of the invention. The human antibody light chain includes two types of genes, i.e., the kappa/K chain gene and the lambda/L chain gene. In one embodiment, the HAC comprises genes encoding both kappa and lambda, in one or more copies. The variable region and constant region of the kappa chain are positioned at 2p11.2-2p12 of the human chromosome 2, and the lambda chain forms a cluster positioned at 22q11.2 of the human chromosome 22. Therefore, in one embodiment, the HAC vectors of the invention comprise a human chromosome 2 fragment containing the kappa chain gene cluster of the 2p11.2-2p12 region. In another embodiment, the HAC vectors of the present invention comprise a human chromosome 22 fragment containing the lambda chain gene cluster of the 22q11.2 region.

In another embodiment, the HAC vector comprises at least one gene encoding a human antibody surrogate light chain. The gene encoding a human antibody surrogate light chain refers to a gene encoding a transient antibody light chain which is associated with an antibody heavy chain produced by a gene reconstitution in the human pro-B cell to constitute the pre-B cell receptor (preBCR). Any suitable human antibody surrogate light chain encoding gene can be used, including but not limited to the VpreB1 (SEQ ID NO: 7), VpreB3 (SEQ ID NO: 8) and λ5 (also known as IgLL1, SEQ ID NO: 9) human antibody surrogate light chains, and combinations thereof. The VpreB gene and the λ5 gene are positioned within the human antibody lambda chain gene locus at 22q11.2 of the human chromosome 22. Therefore, in one embodiment the HAC may comprise the 22q11.2 region of human chromosome 22 containing the VpreB gene and the λ5 gene. The human VpreB gene of the present invention provides either or both of the VpreB1 gene (SEQ ID NO: 7) and the VpreB3 (SEQ ID NO: 8) gene and in one embodiment provides both of the VpreB1 gene and the VpreB3 gene.

In yet another embodiment, the HAC vector comprises a gene encoding an ungulate-derived IgM heavy chain constant region. In this embodiment, the IgM heavy chain constant region is expressed as a chimera with the human IgM antibody heavy chain variable region. Any suitable ungulate IgM heavy chain antibody constant region encoding nucleic acid can be used, including but not limited to bovine IgM, (SEQ ID NO: 10), horse IgM, (SEQ ID NO: 11), sheep IgM, (SEQ ID NO: 12), and pig IgM, (SEQ ID NO: 13). In one embodiment, the chimeric IgM comprises the sequence in SEQ ID NO: 14. Pre-BCR/BCR signaling through the IgM heavy chain molecule promotes proliferation and development of the B cell by interacting with the B cell membrane molecule Ig-alpha/Ig-beta to cause a signal transduction in cells. Transmembrane region and/or other constant region of IgM are considered to have important roles in the interaction with Ig-alpha/Ig-beta for signal transduction. Examples of the IgM heavy chain constant regions include nucleic acids encoding constant region domains such as CH1, CH2, CH3, and CH4, and the B-cell transmembrane and cytoplasmic domains such as TM1 and TM2. The nucleic acid encoding an ungulate-derived IgM heavy chain constant region which is comprised in the human artificial chromosome vector of the invention is not particularly limited so long as the region is in a range which may sufficiently induce the signal of the B-cell receptor or B-cell proliferation/development in the above-described IgM heavy chain constant region. In one embodiment, the nucleic acid encoding an ungulate-derived IgM heavy chain constant region provides a transmembrane and cytoplasmic TM1 domain and TM2 domain derived from an ungulate, and in other embodiments encodes the ungulate-derived CH2 domain, CH3 domain, CH4 domain, TM1 domain, and TM2 domain or the ungulate-derived CH1 domain, CH2 domain, CH3 domain, CH4 domain, TM1 domain, and TM2 domain.

In one embodiment, the gene encoding the IgM heavy chain constant region of the bovine is a gene encoding a bovine IgM heavy chain constant region which is included in an IGHM region at which a bovine endogenous IgM heavy chain gene is positioned (derived from IGHM) or a gene encoding a bovine IgM heavy chain constant region in an IGHML1 region (derived from IGHML1). In another embodiment, the gene encoding a bovine IgM heavy chain constant region is included in the IGHM region.

In a further embodiment, the HAC comprises a gene encoding a human antibody heavy chain comprises a gene encoding a human heavy chain (for example, a human IgG heavy chain, such as an IgG1 heavy chain), and wherein a transmembrane domain and an intracellular domain of a constant region of the human heavy chain gene are replaced with a transmembrane domain and an intracellular domain of an ungulate-derived heavy chain (for example, an ungulate IgG heavy chain, such as an IgG1 heavy chain), constant region gene. In one embodiment, gene encoding the transmembrane domain and the intracellular domain of an ungulate-derived (such as bovine) IgG (such as IgG1) heavy chain constant region are used to replace the corresponding regions of the human IgG heavy chain gene. In another embodiment, the gene encoding the TM1 and TM2 domains of an ungulate-derived (such as bovine) IgG (such as IgG1) heavy chain constant region are used to replace the corresponding regions of the human IgG heavy chain gene. In another embodiment, the gene encoding the one or more of the CH1-CH4 domains and/or the TM1 and TM2 domains of an ungulate-derived (such as bovine) IgG (such as IgG1) heavy chain constant region are used to replace the corresponding regions of the human IgG heavy chain gene.

In one embodiment, avidity of a molecular interaction between two molecules can be measured via different techniques, such as the well the known surface plasmon resonance (SPR) biosensor technique where one molecule is immobilized on the biosensor chip and the other molecule is passed over the immobilized molecule under flow conditions yielding kₒₙ, k_{off} measurements and hence avidity values.

In one embodiment, the population of human immunoglobulins may be said to bind a target polypeptide disclosed herein or a fragment or variant thereof with an avidity of less than or equal to 10⁻¹ 1/sec, 10⁻² 1/sec, or 10⁻³ 1/sec. In one embodiment, the population of human immunoglobulins of the invention may be said to bind a target polypeptide disclosed herein or a fragment or variant thereof with an avidity less than or equal to 10⁻⁴ 1/sec, 10⁻⁵ 1/sec, 10⁻⁶ 1/sec, or 10⁻⁷ 1/sec.

In one embodiment, the population of human immunoglobulins comprises an avidity for PD-L1 of at least 120%, at least 110%, at least 100%, at least 90%, at least 80%, at least 70%, at least 60%, at least 50%, or at least 40%, that of ATCC Deposit No. PTA-127159.

The disclosure further provides transgenic ungulates comprising a HAC vector according to any embodiment or combination of embodiments of the disclosure. The transgenic ungulate comprising the HAC vector of the present invention refers to an animal into which the human artificial chromosome vector of the present invention is introduced. The transgenic ungulate having the HAC of the present invention is not particularly limited so long as the animal is a transgenic ungulate in which the human artificial chromosome fragment may be introduced into a cell thereof, and any non-human animals, for example, ungulates such as cows, horses, goats, sheep, and pigs; and the like may be used. In one aspect, the transgenic ungulate is a bovine. A transgenic ungulate having the HAC vector of the present invention may be constructed, for example, by introducing the HAC vector of the present disclosure into an oocyte of a host animal using any suitable technique, such as those described herein. The HAC vector of the present invention may, for example, be introduced into a somatic cell derived from a host ungulate by a microcell fusion method. Thereafter, the animal having the HAC vector may be constructed by transplanting a nucleus or chromatin agglomerate of the cell into an oocyte and transplanting the oocyte or an embryo to be formed from the oocyte into the uterus of a host animal to give birth. It may be confirmed by a method of Kuroiwa et al. (Kuroiwa et al., Nature Biotechnology, 18, 1086-1090, 2000 and Kuroiwa et al., Nature Biotechnology, 20, 889-894) whether an animal constructed by the above method has the human artificial chromosome vector.

The disclosure further provides transgenic ungulates comprising genes integrated into its genome encoding:
(a) one or more human antibody heavy chains, wherein each gene encoding an antibody heavy chain is operatively linked to a class switch regulatory element;
(b) one or more human antibody light chains; and
(c) one or more human antibody surrogate light chains, and/or an ungulate-derived IgM heavy chain constant region;
wherein at least one class switch regulatory element of the genes encoding the one or more human antibody heavy chains is replaced with an ungulate-derived class switch regulatory element.

In such embodiments, the transgenic ungulate may comprise any embodiment or combination of embodiments of the nucleic acids as described herein for the HAC, but rather than being present in a HAC, they are integrated into a chromosome of the ungulate.

The disclosure further provides a method of producing a human antibody, comprising: (a) administering PD-L1, or other target antigen of the disclosure, to the transgenic ungulate of any embodiment or combination of embodiments of the disclosure to produce and accumulate a population of human immunoglobulins specific to PD-L1 (or T cells, B cells, and/or monocytes) in the serum or plasma of the ungulate; and optionally (b) isolating, recovering, and/or purifying the population of human immunoglobulins specific to the PD-L1 (or T cells, B cells, and/or monocytes) from the serum or plasma of the ungulate.

The polyclonal serum or plasma, or human immunoglobulin purified from the polyclonal serum or plasma, may be used as an immunoglobulin product for cancer.

In a variation, the disclosure provides a method of recovering the protein sequence of a human antibody comprises: (i) isolating lymphocytes from the transgenic ungulate; (ii) generating a human monoclonal antibody producing hybridoma from the lymphocytes; and (iii) recovering human monoclonal antibody specific to the antigen from the hybridoma. In another embodiment, the lymphocytes from the transgenic ungulate are isolated from lymph nodes of the transgenic ungulate. In a further embodiment the transgenic ungulate is hyperimmunized with the target antigen.

A PD-L1-specific human immunoglobulin (such as PD-L1-specific human immunoglobulin) may be produced by immunizing the transgenic ungulate having the HAC vector with human PD-L1, or another antigen of the disclosure, to produce the PD-L1-specific human immunoglobulin in the serum or plasma of the transgenic ungulate and recovering the PD-L1-specific human immunoglobulin from the serum or plasma of the transgenic ungulate.

Examples of methods for detecting and measuring the PD-L1-specific human immunoglobulin in a composition include a binding assay by an enzyme-linked immunosorbent assay, and the like. The binding amount of a human immunoglobulin may be measured by incubating the composition comprising the human immunoglobulin with cells (e.g., T cells, B cells and/or monocytes, or recombinant protein antigen(s)), and then using an antibody specifically recognizing human immunoglobulin.

In a variation, the methods of the disclosure are used to generate a monoclonal antibody. Methods of preparing and utilizing various types of antibodies are well-known to those of skill in the art and would be suitable in practicing the present invention (see, for example, Harlow, et al. Antibodies: A Laboratory Manual, Cold Spring Harbor Laboratory, 1988; Kohler and Milstein, Nature 256:495 (1975)). An example of a preparation method for hybridomas comprises the following steps of: (1) immunizing a transgenic ungulate with a recombinant PD-L1; (2) collecting antibody-producing cells from the transgenic ungulate (i.e. from lymph nodes); (3) fusing the antibody-producing cells with myeloma cells; (4) selecting hybridomas that produce a monoclonal antibody specific to a recombinant PD-L1 from the fused cells obtained in the above step; and optionally (5) selecting a hybridoma that produces a monoclonal antibody specific to PD-L1 from the selected hybridomas.

In embodiments of the methods of producing polyclonal immunoglobulin specific for PD-L1 (such as PD-L1-specific human immunoglobulin), the transgenic ungulate produces human polyclonal immunoglobulin specific for PD-L1. The method may comprise collecting the polyclonal serum and/or polyclonal plasma from the transgenic ungulate. In some embodiments, the ungulate is a bovine. In some embodiments, the polyclonal immunoglobulin composition comprises a population of fully human immunoglobulins. In some embodiments, the polyclonal immunoglobulin composition comprises a population of fully human immunoglobulins, substantially human immunoglobulins.

Some embodiments of the methods of the disclosure, and related compositions, have the surprising advantage that the PD-L1-specific immunoglobulins (such as PD-L1-specific human immunoglobulin) are produced in high yield, in high purity, and/or as a high percentage of total immunoglobulin present in the serum or plasma of the transgenic ungulate. Furthermore, some embodiments produce PD-L1-specific immunoglobulins having glycans that comprise at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95% or higher percentage of fucosylated glycans. Furthermore, some embodiments produce PD-L1-specific immunoglobulins having at most about the same ADCC or CDC activity as a reference immunoglobulin preparation, e.g. human-derived immunoglobulin.

In some embodiments, the population of human immunoglobulins binds FcγRI with a K_{D} of 15 nM or greater. In some embodiments, the population of human immunoglobulins binds FcγRIIa with a K_{D} of 500 nM or greater. In some embodiments, the population of human immunoglobulins binds FcγRIIb/c with a K_{D} of 1 μM or greater. In some embodiments, the population of human immunoglobulins binds FcγRIIIa with a K_{D} of 1 μM or greater. In some embodiments, the population of human immunoglobulins binds FcγRIIIa with a K_{D} of 1 nM or greater.

In some embodiments of the methods and compositions of the disclosure, the polyclonal serum or polyclonal plasma comprises at least 0.1%, at least 0.2%, at least 0.3%, at least 0.4%, at least 0.5%, at least 0.6%, at least 0.7%, at least 0.8%, at least 0.9%, at least 1%, at least 1.1%, at least 1.2%, at least 1.3%, at least 1.4%, at least 1.5%, at least 1.6%, at least 1.7%, at least 1.8%, at least 1.9%, at least 2%, at least 2.1%, at least 2.2%, at least 2.3%, at least 2.4%, at least 2.5%, at least 2.6%, at least 2.7%, at least 2.8%, at least 2.9%, at least 3%, at least 3.1%, at least 3.2%, at least 3.3%, at least 3.4%, at least 3.5%, at least 3.6%, at least 3.7%, at least 3.8%, at least 3.9%, at least 4%, at least 4.1%, at least 4.2%, at least 4.3%, at least 4.4%, at least 4.5%, at least 4.6%, at least 4.7%, at least 4.8%, at least 4.9%, at least 5%, at least 5.1%, at least 5.2%, at least 5.3%, at least 5.4%, at least 5.5%, at least 5.6%, at least 5.7%, at least 5.8%, at least 5.9%, at least 5.9%, at least 6.0%, at least 6.1%, at least 6.2%, at least 6.3%, at least 6.4%, at least 6.5%, at least 6.6%, at least 6.7%, at least 6.8%, at least 6.9%, at least 7.0%, at least 7.1%, at least 7.2%, at least 7.3%, at least 7.4%, at least 7.5%, at least 7.6%, at least 7.7%, at least 7.8%, at least 7.9%, at least 8.0%, at least 8.1%, at least 8.2%, at least 8.3%, at least 8.4%, at least 8.5%, at least 8.6%, at least 8.7%, at least 8.8%, at least 8.8%, at least 9.0%, at least 9.1%, at least 9.2%, at least 9.3%, at least 9.4%, at least 9.5%, at least 9.6%, at least 9.7%, at least 9.8%, at least 9.8%, at least 9.9%, or at least 10% fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 0.1-0.6%, 0.2-0.7%, 0.3-0.8%, 0.4-0.9%, 0.5-1%, 0.6-1.1%, 0.7-1.2%, 0.8-1.3%, 0.9-1.4%, 1-1.5%, 1.1-1.6%, 1.2-1.7%, 1.3-1.8%, 1.4-1.9%, 1.5-2%, 1.6-2.1%, 1.7-2.2%, 1.8-2.3%, 1.9-2.4%, 2-2.5%, 2.1-2.6%, 2.2-2.7%, 2.3-2.8%, 2.4-2.9%, 2.5-3%, 2.6-3.1%, 2.7-3.2%, 2.8-3.3%, 2.9-3.4%, 3-3.5%, 3.1-3.6%, 3.2-3.7%, 3.3-3.8%, 3.4-3.9%, 3.5-4%, 3.6-4.1%, 3.7-4.2%, 3.8-4.3%, 3.9-4.4%, 4-4.5%, 4.1-4.6%, 4.2-4.7%, 4.3-4.8%, 4.4-4.9%, 4.5-5%, 4.6-5.1%, 4.7-5.2%, 4.8-5.3%, 4.9-5.4%, 5-5.5%, 5.1-5.6%, 5.2-5.7%, 5.3-5.8%, 5.4-5.9%, 5.5-6%, 5.6-6.1%, 5.7-6.2%, 5.8-6.3%, or 5.9-6.4% fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 0-0.5%, 0.5-1%, 1-1.5%, 1.5-2%, 2-2.5%, 2.5-3%, 3-3.5%, 3.5-4% 4-4.5%, 4.5-5%, 5-5.5%, 5.5-6%, 6-6.5%, 6.5-7%, 7-7.5% 7.5-8%, 8-8.5%, 8.5-9%, 9-9.5% 9.5-10% or greater fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 0-1%, 1-2%, 2-3%, 3-4%, 4-5%, 5-6%, 6-7%, 7-8%, 8-9%, 9-10%, or greater fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 0-5%, 5-10%, 10-15%, 15-20%, 20-25%, 25-30%, 30-35%, 35-40%, or greater fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 0-5%, 5-10%, 10-15%, 15-20%, 20-25%, 25-30%, 30-35%, 35-40%, or greater fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises at least 1%, at least 2%, at least 3%, at least 4%, at least 5%, at least 6%, at least 7%, at least 8%, at least 9%, or at least 10% fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 1-4%, 2-5%, 3-6%, 4-7%, 5-8%, 6-9%, or 7-10% fully human (or substantially human) immunoglobulin by mass of total immunoglobulin in the polyclonal serum or polyclonal plasma.

In some embodiments of the methods and compositions of the disclosure, the polyclonal immunoglobulin comprises at least 0.1%, at least 0.2%, at least 0.3%, at least 0.4%, at least 0.5%, at least 0.6%, at least 0.7%, at least 0.8%, at least 0.9%, at least 1%, at least 1.1%, at least 1.2%, at least 1.3%, at least 1.4%, at least 1.5%, at least 1.6%, at least 1.7%, at least 1.8%, at least 1.9%, at least 2%, at least 2.1%, at least 2.2%, at least 2.3%, at least 2.4%, at least 2.5%, at least 2.6%, at least 2.7%, at least 2.8%, at least 2.9%, at least 3%, at least 3.1%, at least 3.2%, at least 3.3%, at least 3.4%, at least 3.5%, at least 3.6%, at least 3.7%, at least 3.8%, at least 3.9%, at least 4%, at least 4.1%, at least 4.2%, at least 4.3%, at least 4.4%, at least 4.5%, at least 4.6%, at least 4.7%, at least 4.8%, at least 4.9%, at least 5%, at least 5.1%, at least 5.2%, at least 5.3%, at least 5.4%, at least 5.5%, at least 5.6%, at least 5.7%, at least 5.8%, at least 5.9%, at least 5.9%, at least 6.0%, at least 6.1%, at least 6.2%, at least 6.3%, at least 6.4%, at least 6.5%, at least 6.6%, at least 6.7%, at least 6.8%, at least 6.9%, at least 7.0%, at least 7.1%, at least 7.2%, at least 7.3%, at least 7.4%, at least 7.5%, at least 7.6%, at least 7.7%, at least 7.8%, at least 7.9%, at least 8.0%, at least 8.1%, at least 8.2%, at least 8.3%, at least 8.4%, at least 8.5%, at least 8.6%, at least 8.7%, at least 8.8%, at least 8.8%, at least 9.0%, at least 9.1%, at least 9.2%, at least 9.3%, at least 9.4%, at least 9.5%, at least 9.6%, at least 9.7%, at least 9.8%, at least 9.8%, at least 9.9%, or at least 10% fully human (or substantially human) immunoglobulin by mass of total immunoglobulin in the polyclonal immunoglobulin.

In some embodiments of the methods and compositions of the disclosure, the polyclonal immunoglobulin comprises 0.1-0.6%, 0.2-0.7%, 0.3-0.8%, 0.4-0.9%, 0.5-1%, 0.6-1.1%, 0.7-1.2%, 0.8-1.3%, 0.9-1.4%, 1-1.5%, 1.1-1.6%, 1.2-1.7%, 1.3-1.8%, 1.4-1.9%, 1.5-2%, 1.6-2.1%, 1.7-2.2%, 1.8-2.3%, 1.9-2.4%, 2-2.5%, 2.1-2.6%, 2.2-2.7%, 2.3-2.8%, 2.4-2.9%, 2.5-3%, 2.6-3.1%, 2.7-3.2%, 2.8-3.3%, 2.9-3.4%, 3-3.5%, 3.1-3.6%, 3.2-3.7%, 3.3-3.8%, 3.4-3.9%, 3.5-4%, 3.6-4.1%, 3.7-4.2%, 3.8-4.3%, 3.9-4.4%, 4-4.5%, 4.1-4.6%, 4.2-4.7%, 4.3-4.8%, 4.4-4.9%, 4.5-5%, 4.6-5.1%, 4.7-5.2%, 4.8-5.3%, 4.9-5.4%, 5-5.5%, 5.1-5.6%, 5.2-5.7%, 5.3-5.8%, 5.4-5.9%, 5.5-6%, 5.6-6.1%, 5.7-6.2%, 5.8-6.3%, or 5.9-6.4% fully human (or substantially human) immunoglobulin by mass of total immunoglobulin in the polyclonal immunoglobulin.

In some embodiments of the methods and compositions of the disclosure, the polyclonal immunoglobulin comprises 0-0.5%, 0.5-1%, 1-1.5%, 1.5-2%, 2-2.5%, 2.5-3%, 3-3.5%, 3.5-4%, 4-4.5%, 4.5-5%, 5-5.5%, 5.5-6%, 6-6.5%, 6.5-7%, 7-7.5%, 7.5-8%, 8-8.5%, 8.5-9%, 9-9.5%, 9.5-10% or greater fully human (or substantially human) immunoglobulin by mass of total immunoglobulin in the polyclonal immunoglobulin.

In some embodiments of the methods and compositions of the disclosure, the polyclonal immunoglobulin comprises 0-1%, 1-2%, 2-3%, 3-4%, 4-5%, 5-6%, 6-7%, 7-8%, 8-9%, 9-10%, or greater fully human (or substantially human) immunoglobulin by mass of total immunoglobulin in the polyclonal immunoglobulin.

In some embodiments of the methods and compositions of the disclosure, the polyclonal immunoglobulin comprises 0-5%, 5-10%, 10-15%, 15-20%, 20-25%, 25-30%, 30-35%, 35-40%, or greater fully human (or substantially human) immunoglobulin by mass of total immunoglobulin in the polyclonal immunoglobulin.

In some embodiments of the methods and compositions of the disclosure, the polyclonal immunoglobulin comprises 0-5%, 5-10%, 10-15%, 15-20%, 20-25%, 25-30%, 30-35%, 35-40%, or greater fully human (or substantially human) immunoglobulin by mass of total immunoglobulin in the polyclonal immunoglobulin.

In some embodiments of the methods and compositions of the disclosure, the polyclonal immunoglobulin comprises at least 1%, at least 2%, at least 3%, at least 4%, at least 5%, at least 6%, at least 7%, at least 8%, at least 9%, or at least 10% fully human (or substantially human) immunoglobulin by mass of total immunoglobulin in the polyclonal immunoglobulin.

In some embodiments of the methods and compositions of the disclosure, the polyclonal immunoglobulin comprises 1-4%, 2-5%, 3-6%, 4-7%, 5-8%, 6-9%, or 7-10% fully human (or substantially human) immunoglobulin by mass of total immunoglobulin in the polyclonal immunoglobulin.

In some embodiments of the methods and compositions of the disclosure, the polyclonal immunoglobulin comprises at least 5% fully human immunoglobulin by mass of total immunoglobulin in the polyclonal immunoglobulin.

In some embodiments of the methods and compositions of the disclosure, the polyclonal immunoglobulin comprises 2% to 5% fully human immunoglobulin by mass of total immunoglobulin in the polyclonal immunoglobulin.

In some embodiments, the ungulate-derived polyclonal immunoglobulin comprises “chimeric” human immunoglobulin having a human heavy chain and an ungulate kappa light chain (termed “cIgG”). In some embodiments, the polyclonal immunoglobulin comprises less than about 0.5%, less than about 0.75%, less than about 1.0%, less than about 1.25%, less than about 1.5%, less than about 1.75%, less than about 2.0%, less than about 2.25%, less than about 2.5%, less than about 2.75%, less than about 3.0%, less than about 3.25%, less than about 3.5%, less than about 3.75%, or less than about 4.0% cIgG as a percent of total protein concentration. In some embodiments, the polyclonal immunoglobulin comprises about 0.5% to about 1.0%, about 1.0% to about 1.5%, about 1.5% to about 2.0%, about 1.5% to about 2.0%, about 2.0% to about 2.5%, or about 2.5% to about 3.0% cIgG as a percent of total protein concentration. In some embodiments, the polyclonal immunoglobulin comprises about 0.5% to about 1.0%, about 1.0% to about 2.0%, or about 1.0 to about 3.0% cIgG as a percent of total protein concentration.

In some embodiments, the polyclonal immunoglobulins of the disclosure are less potent in a complement-dependent cytotoxicity (CDC) assay than a reference product (e.g. human-derived polyclonal immunoglobulin). In some embodiments, the polyclonal immunoglobulins of the disclosure are at most about 5%, at most about 10%, at most about 25%, at most about 50%, at most about 100%, at most about 150%, or more at most about 200% potent in a complement-dependent cytotoxicity (CDC) assay than a reference product (e.g. human-derived polyclonal immunoglobulin).

In some embodiments, the polyclonal immunoglobulins of the disclosure generate lower toxicity towards CD8+ cells than a reference product (e.g. human-derived polyclonal immunoglobulin. In some embodiments, the polyclonal immunoglobulins of the disclosure are at most about 5%, at most about 10%, at most about 25%, at most about 50%, at most about 100%, at most about 150%, or at most about 200% more potent in CD8+ cell killing assay than a reference product (e.g. human-derived polyclonal immunoglobulin).

In some embodiments, the polyclonal immunoglobulins of the disclosure generated lower rates of CD4+ T cell apoptosis than a reference product (e.g. human-derived polyclonal immunoglobulin. In some embodiments, the polyclonal immunoglobulins of the disclosure are at least about 5%, at least about 10%, at least about 25%, at least about 50%, at least about 100%, at least about 150%, or at least about 200% less toxic in a CD4+ cell apoptosis assay than a reference product (e.g. human-derived polyclonal immunoglobulin).

In some embodiments, the polyclonal immunoglobulins of the disclosure better preserves T_{reg} to conventional T cell rations than a reference product (e.g. human-derived polyclonal immunoglobulin. In some embodiments, the polyclonal immunoglobulins of the disclosure are at least about 5%, at least about 10%, at least about 25%, at least about 50%, at least about 100%, at least about 150%, or at least about 200% less toxic to T_{reg} cells than a reference product (e.g. human-derived polyclonal immunoglobulin).

In some embodiments of the methods and compositions of the disclosure, the population of fully human immunoglobulins (or substantially human) specifically binds PD-L1, or an immunologically similar antigen.

In some embodiments, a genome of the transgenic ungulate comprises a human immunoglobulin locus.

In some embodiments, the transgenic ungulate is immunized 3, 4, 5, or more times.

In some embodiments, the population of fully human or substantially human immunoglobulins are purified from the serum of the transgenic ungulate after immunization.

The disclosure provides methods of providing human polyclonal immunoglobulin specific for PD-L1 (such as PD-L1) treatment to a subject in need thereof, comprising administering to the subject a polyclonal immunoglobulin according to the disclosure. In some embodiments, the method provides an effective amount of human polyclonal immunoglobulin specific for PD-L1 to the subject.

The disclosure provides methods of providing human polyclonal immunoglobulin specific for PD-L1 (such as PD-L1) treatment to a subject in need thereof, comprising administering to the subject a composition produced by immunizing a transgenic ungulate with PD-L1. In some embodiments, the method provides an effective amount of human polyclonal immunoglobulin specific for PD-L1 to the subject.

The disclosure provides methods of providing human polyclonal immunoglobulin specific for PD-L1 (such as PD-L1) treatment to a subject in need thereof, comprising administering to the subject a polyclonal immunoglobulin produced according to the disclosure. In some embodiments, the method provides an effective amount of human polyclonal immunoglobulin specific for PD-L1 to the subject.

The disclosure further provides pharmaceutical compositions, comprising a population of fully human or substantially human immunoglobulins, and one or more pharmaceutically acceptable excipients. In some embodiments, the population of fully human or substantially human immunoglobulins specifically binds human PD-L1, or antigenic fragment thereof.

In some embodiments, the pharmaceutical composition comprises at least about 1 mg/mL, at least about 50 mg/mL, at least about 100 mg/mL, or at least about 1,000 mg/mL of fully human or substantially human immunoglobulin. In some embodiments, the pharmaceutical composition comprises at least about 100 μg/mL, at least about 250 μg/mL, at least about 500 μg/mL, at least about 750 μg/mL, or at least about 1,000 μg/mL of fully human or substantially human immunoglobulin.

In some embodiments, the fully human or substantially human immunoglobulin is produced in an ungulate. In some embodiments, the ungulate is a bovine.

In some embodiments, the pharmaceutical composition comprises at least 5% fully human immunoglobulin by mass of total immunoglobulin in the pharmaceutical composition.

In some embodiments, the pharmaceutical composition comprises 2% to 5% fully human immunoglobulin by mass of total immunoglobulin in the pharmaceutical composition.

### EXAMPLES

The following specific examples are to be construed as merely illustrative, and not limitative of the remainder of the disclosure in any way whatsoever.

### Example 1

### Generation and Characterization of Rabbit Anti-Human PD-L1 Polyclonal Antibodies

Generation of Human PD-L1 Fusion Protein and Rabbit Anti-Human PD-L1 IgG

This Example describes generating and testing in vivo a rabbit polyclonal antibody (pAb) against human PD-L1.

To generate a PD-L1 antigen to use for rabbit immunizations, the pFUSE-His-HRV3C-PDL1(19-239) expression construct was synthesized. The pFUSE-His-HRV3C-PDL1(19-239) plasmid encoded the modified serum albumin signal peptide followed by 9 His residues and the HRV3C cleavage site. Sequence encoding amino acids 19-239 of the extracellular domain (ECD) of PD-L1 (SEQ ID NO: 15) was clone 3′ of the HRV3C cleavage site to generate a fusion polypeptide having the sequence set forth in SEQ ID NO: 16. The pFUSE-PDL1(1-239)-HRCV3-His was transfected into Expi 293 cells, and the culture supernatant was collected. The HisPur Ni-NTA spin columns were used to purify the antigen. The HRV3C protease was used to remove the His-tag from the PD-L1 ECD. The His-tagged HRV3C was removed by passing through a nickel column. The antigen was referred to as SDSU-PDL1-ECD.

The rabbit immunization protocol was designed to mimic the hyperimmunization strategy used for the transchromosomic (Tc) bovine. Two rabbits were hyperimmunized with 0.2 mg per dose of recombinant SDSU-PDL1-ECD antigen formulated with adjuvant formulation (ISA-206 plus Quil A) for 2 vaccinations (V1 and V2). The rabbits were hyperimmunized with 0.5 mg of SDSU-PDL1-ECD for V3.

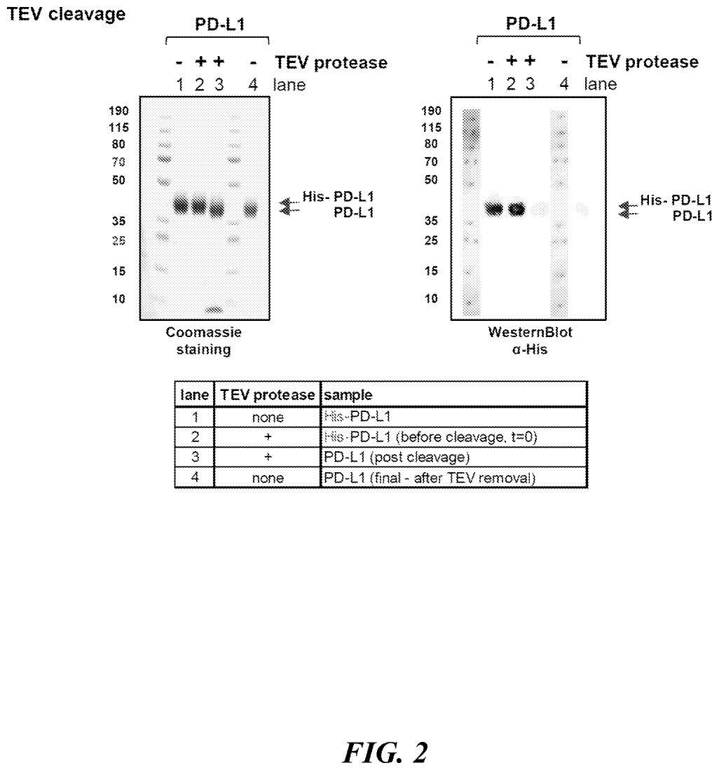

To generate a PD-L1-ECD antigen used for V4-V5 rabbit immunizations, sequence encoding the human PD-L1 ECD (amino acids 19-238; SEQ ID NO: 15) containing the IgV-like and IgC2-type domains was cloned into the pcDNA3.4 expression vector. The endogenous signal sequence of PD-L1 was replaced with the immunoglobulin kappa-chain signal sequence. Ten histidine amino acids followed by a TEV protease site were located between the signal sequence and the extracellular domains of PD-L1, resulting in a fusion polypeptide having the sequence set forth in SEQ ID NO: 17. The expression plasmid was transfected into Expi293 cells, and the supernatant was collected for purification of the secreted protein 6 days post-transfection. The supernatant was passed over a HisTrap column with linear gradient from 20-500 mM Imidazole. After TEV protease cleavage, the protease was removed using a HisTrap column. For polishing, a HiLoad Superdex200 XK 50/70 column was used (FIG. 2), and the antigen is referred to as TMO-PDL1-ECD.

The two rabbits previously immunized with SDSU-PDL1-ECD (SEQ ID NO: 16) for V1-V3 were hyperimmunized with 0.5 mg per dose of recombinant TMO-PDL1-ECD (SEQ ID NO: 17) protein antigen formulated with adjuvant formulation (ISA-206 plus Quil A) for V4 and V5. The rabbit anti-human PD-L1 pAb were purified from pooled V5 sera.

Binding of Rabbit Anti-Human PD-L1 pAb to PD-L1 Expressing Cells.

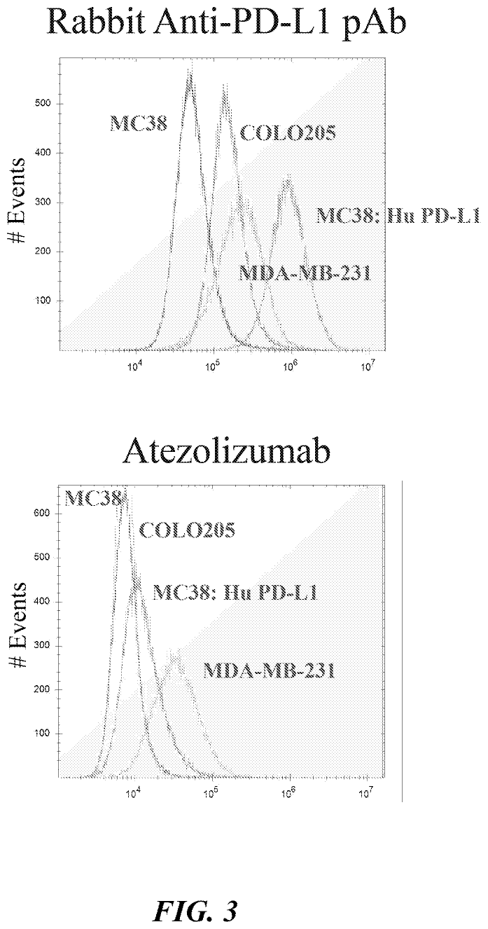

The rabbit anti-human PD-L1 pAb was evaluated for binding to PD-L1 on cell lines. The human breast cancer cell line MDA-MB-231 (expresses high levels of PD-L1), human colon cancer COLO205 (low expressing PD-L1), and MC38-hPD-L1 (colon cancer mouse cell line expressing the human PD-L1 gene) were chosen for flow cytometry analysis (FIG. 3). Cell lines were incubated with the humanized mAb Atezolizumab anti-PD-L1 as a positive control or the rabbit anti-human PD-L1 pAb. A PE-labelled anti-human IgG or Alexa Fluor 488 labelled anti-rabbit IgG, respectively, was added, and flow cytometry analysis was performed. Atezolizumab had the strongest binding to MDA-MB-231 cells which express high levels of PD-L1. Anti-PD-L1 pAb had the strongest binding to MC38-hPD-L1 cells expressing human PD-L1 (FIG. 3). The pAb bound 100-fold greater to MC38-hPD-L1 cells and 10-fold higher to MDA-MB-231 cells compared to Atezolizumab. The purified rabbit pAb demonstrated higher targeted binding to PD-L1 than the commercial anti-PDL1 mAb antibody Atezolizumab.

Complement-Dependent Cytotoxicity (CDC) Activity of Rabbit Anti-Human PD-L1 pAb.

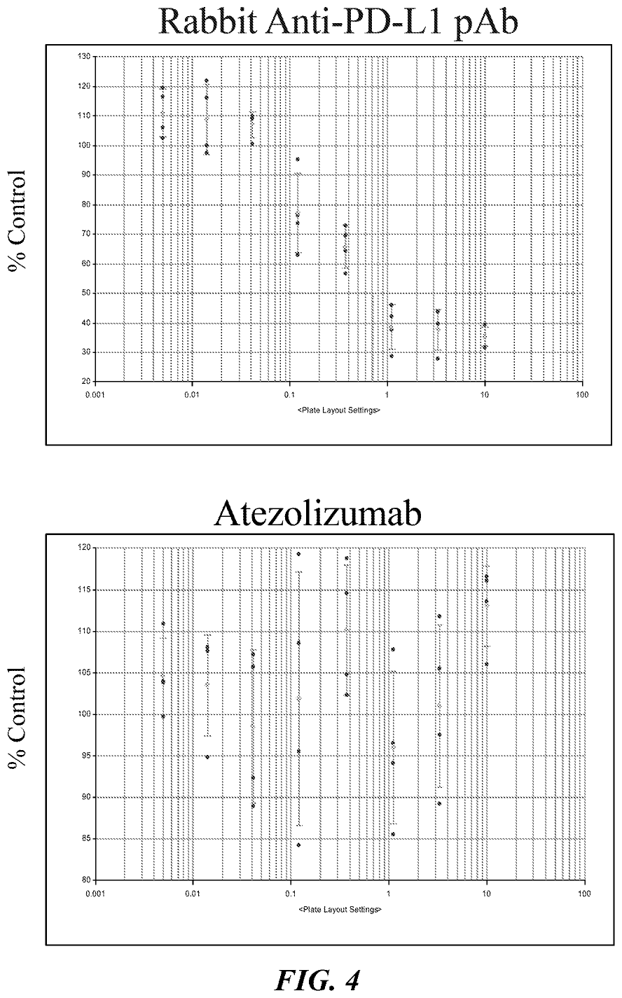

To determine whether rabbit anti-human PD-L1 pAb have complement activation, a complement mediated cytotoxicity analysis was performed. Briefly, the MC38-hPD-L1 cells were seeded into 96-well plates and incubated at 37° C. for 7 days. Serial dilutions of Atezolizumab and anti-PD-L1 pAb from 10 μg/mL to 0.005 μg/mL were added to the cells and incubated for 1 hour. Ice cold complement was added to the wells, except not to the no-complement controls. The plates were incubated at 37° C. for 72 hours. Cell viability was determined by the Promega CellTiter-Glo assay. Anti-PD-L1 pAb demonstrated CDC activity with IC₅₀ of 0.72 μg/ml. Atezolizumab is a humanized IgG1 Fc-deficient mAb which did not demonstrate CDC activity (FIG. 4).

Preclinical Syngeneic Mouse Model

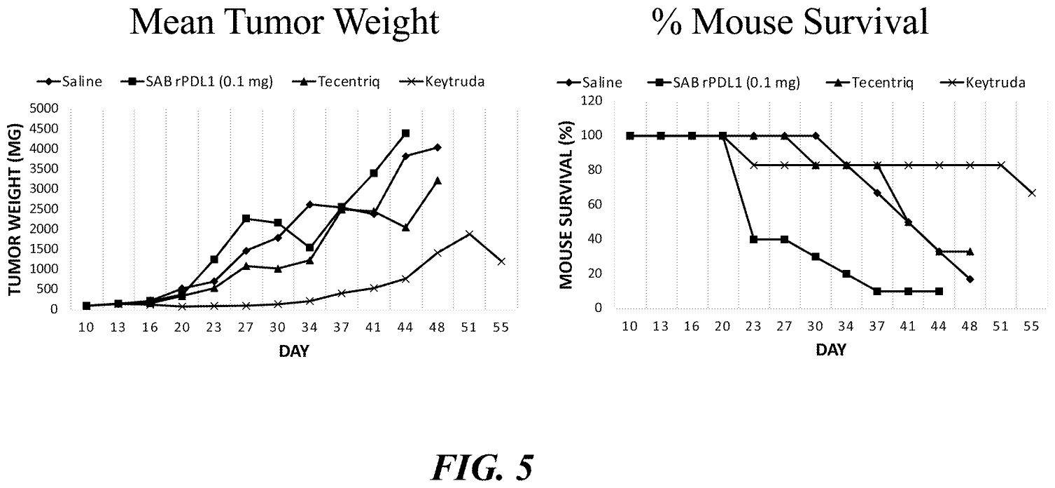

The in vitro characterization assays demonstrated that rabbit anti-human PD-L1 pAb binds to PD-L1 on cells and has complement activation. A preclinical mouse model was used to determine whether anti-PD-L1 pAb can reduce tumor burden in mice. An PD-1/L1 C57BL/6 transgenic mouse that expresses both the human PD-1 and PD-L1 genes with the murine genes knocked-out was generated. The MC38 colon carcinoma cell line was originally derived from methylcholanthrene-induced C57BL6 murine colon adenocarcinoma cells. MC38 cells were genetically modified to stably express human PD-L1, referred to as MC38-hPD-L1 cells. A syngeneic mouse model allows analysis of the immune system's response to the tumor. Eight- to nine-week-old C57BL6 PD-1/PD-L1 knock in mice were injected subcutaneously in the right flank with 5×10⁵ MC38-hPD-L1 colon tumor cells (Day 0). The mice were placed into four treatment groups. Group 1 consisted of 2 male and 3 female C57BL/6 PD-1/PD-L1 knock in mice, and the mice were treated with saline as a control. Group 2 consisted of 5 male and 4 female mice, and the mice were treated with rabbit anti-human PD-L1 IgG (0.1 mg per dose). Atezolizumab (0.1 mg/dose) was given to Group 3, and pembrolizumab (0.1 mg/dose) was used in Group 4. Groups 3 and 4 both contained 1 male and 1 female mouse. Treatment was started when the weights of the tumors were 48 to 126 mg. Tumors were measured twice a week starting on the first day of treatment. Individual tumor weights and percentage of mouse survival were plotted over time for all groups (FIG. 5).

Compared to the saline control, rabbit anti-human PD-L1 pAb decreased tumor weights between 27 and 34 days; however, 0.1 mg/dose had significant toxicity and the mice did not survive (FIG. 5). Atezolizumab did not have a substantial effect on decreasing tumor weight, which suggested that the dose of atezolizumab given to the mice was too low. Pembrolizumab performed very well with 80% of the mice still alive after >50 days.

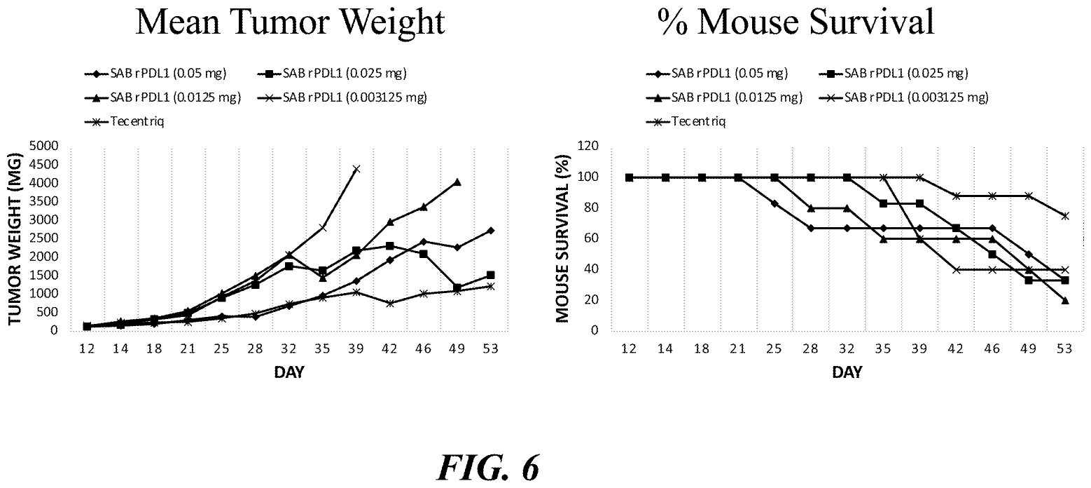

Treating tumor bearing mice with 0.1 mg/dose of rabbit anti-human PD-L1 pAb resulted in significant toxicity, and the mice did not survive. To determine an efficacious dose while decreasing toxicity, an anti-PD-L1 pAb titration analysis was performed. C57BL6 PD-1/PD-L1 knock in mice were injected subcutaneously with MC38-hPD-L1 colon tumor cells. The mice were divided into 5 groups by treatment regimen. Groups 1-4 were treated with 0.05, 0.025, 0.0125 and 0.003125 mg/dose of rabbit anti-human PD-L1 pAb, respectively, and group 5 received 1 mg/dose of atezolizumab. Groups 1 and 2 consisted of 6 mice. Groups 3 and 4 contained 5 mice, and group 5 had 8 mice with both male and female mice being represented in all groups. After tumors were established, mice were treated with rabbit anti-human PD-L1 pAb or atezolizumab on days 12, 14, 18 and 21, and tumor weights were measured 2 times per week (FIG. 6).

Mice that received the lower doses of rabbit anti-human PD-L1 pAb lived longer than mice treated with a higher dose, suggesting that toxicity correlated with the amount of rabbit anti-human PD-L1 pAb. However, tumor reduction occurred in the surviving mice treated with 0.05 mg/dose through day 35 (FIG. 6). The reduction in tumor burden indicates that rabbit anti-human PD-L1 pAb has anti-cancer effects. All mice treated with atezolizumab lived to day 39 and exhibited a low tumor burden.

This Example demonstrates that the cancer killing effects of rabbit anti-human PD-L1 pAb can be harnessed while minimizing the toxicity of the pAb.

### Example 2

### Characterization of Human Anti-Human PD-L1 Polyclonal Immunoglobulin Produced in the Transchromosomic Bovine (TcB) Platform

Generating Human Anti-PD-L1 Polyclonal Antibodies in Transchromosomic (Tc) Bovine

Applicant has developed a transchromosomic (Tc) bovine production system in which bovine Ig genes are knocked-out and a human artificial chromosome (HAC) vector is introduced into the bovine cell to express human polyclonal antibodies. All current therapeutic antibodies used to treat cancer are monoclonal IgG1.

One Tc bovine was immunized twice (V1 and V2) with 2 mg of recombinant TMO-PDL1-ECD (generated as described in Example 1 for hyperimmunization of rabbits, see FIG. 2). Five mg TMO-PDL1-ECD (SEQ ID NO: 17) was administered per dose for V3, V4 and V5 (Table 1).

TABLE 1
Vaccination Interval and Formulation

| Tc Bovine | Vaccination | Vaccine Formulation |
|---|---|---|
| TKO/isKcHACA | V1 to V5 | 2 mg TMO-PDL1-ECD + |
| | with 3-week | ISA-206/Quil A for V1 and V2 |
| | intervals | 5 mg TMO-PDL1-ECD + |
| | | ISA-206/Quil A for V3-V5 |

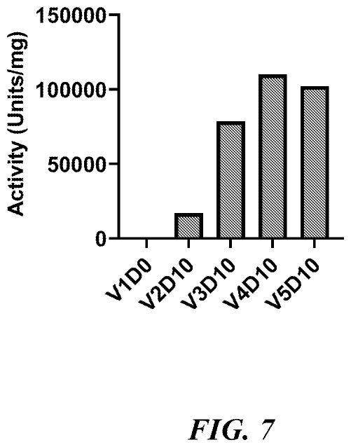

The Tc animal was immunized with TMO-PDL1-ECD vaccine via intramuscular injections on both sides of neck and hind leg regions with equal vaccine volume on each area. Serum was collected from the Tc Bovine over the course of the five immunizations, and ELISA was performed to determine the titer against PD-L1 (FIG. 7). The titer of anti-PD-L1 antibodies increased over the course of vaccinations.

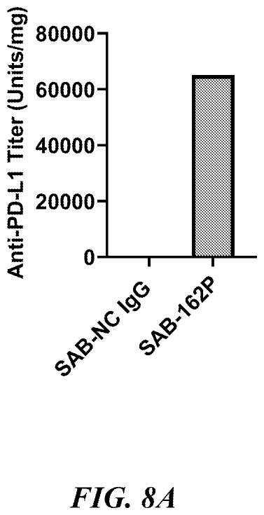

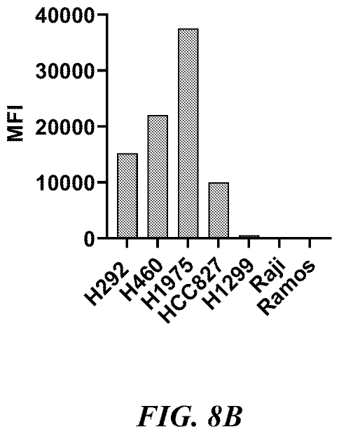

Plasma was collected after V3 to V5, and the anti-PD-L1 fully human IgGs were purified. Briefly, collected plasma was frozen for shipment, thawed, pooled, fractionated by caprylic acid (CA), and clarified by depth filtration. Clarified material containing Tc bovine-derived human immunoglobulin G (IgG) was purified by affinity chromatography using an anti-human IgG affinity column first to bind Tc bovine-derived human IgG (hIgG) and remove bovine plasma proteins (BPP). Second, a low pH treatment for viral inactivation is performed following by using an anti-bovine IgG (bIgG) heavy chain (HC) specific affinity column to further remove residual IgG molecules that contain a bovine HC or Fc of bovine HC. The Tc bovine-derived human IgG fraction was then concentrated and diafiltered prior to a Q Sepharose chromatography polishing step, nanofiltration, final buffer exchange, concentration and sterile filtration. The purified anti-PD-L1 fully human IgGs are referred to as SAB-162P. An indirect ELISA was performed on SAB-162P using Tc Bovine pre-immune plasma as a negative control (FIG. 8A).

Binding of TcB-Derived Anti-PD-L1 Polyclonal Antibodies to Cell Surface PD-L1.

To verify that SAB-162P binds to PD-L1-expressing cancer cell lines, human non-small cell lung carcinoma cell lines that were positive for PD-L1 on the cell surface were studied, including H292, H460, H1975, HCC827, and H1299. For a negative binding control, PD-L1 negative Raji and Ramos lymphoma cells were utilized. SAB-162P was directly conjugated to AF-488, and cell lines were incubated with the fluorescently labeled primary antibodies. Flow cytometry analysis was performed to determine the relative amount of surface PD-L1 by measuring fluorescent intensity levels of the live cells (FIG. 8B). SAB-162P demonstrated targeted binding to PD-L1-expressing H292, H460, H1975 and HCC827. Minimal levels were observed in H1299 cells, and no binding was detected to Raji and Ramos lymphoma cells, which are PD-L1 negative. H1975 had the highest level of cell surface PD-L1 followed by H460, H292, and H1299 had the least amount of the PD-L1 target protein.

Titration of SAB-162P Binding to Human NSCLC Cell Lines

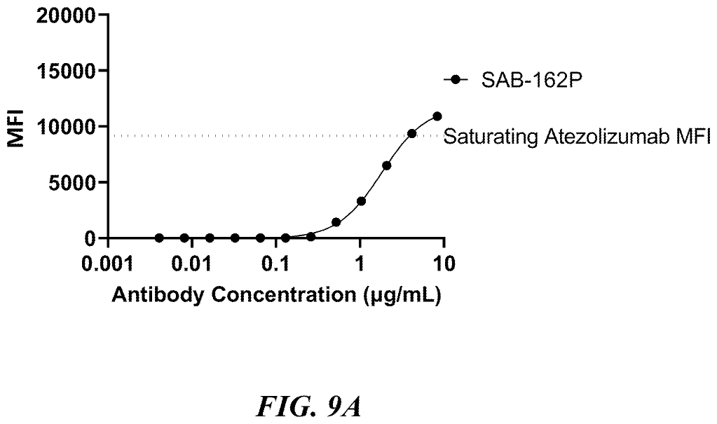

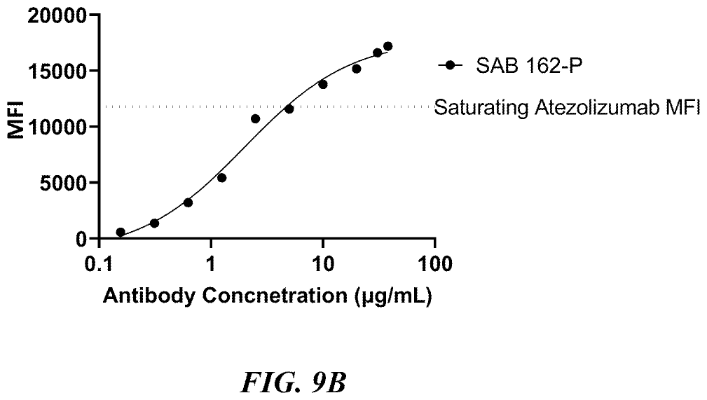

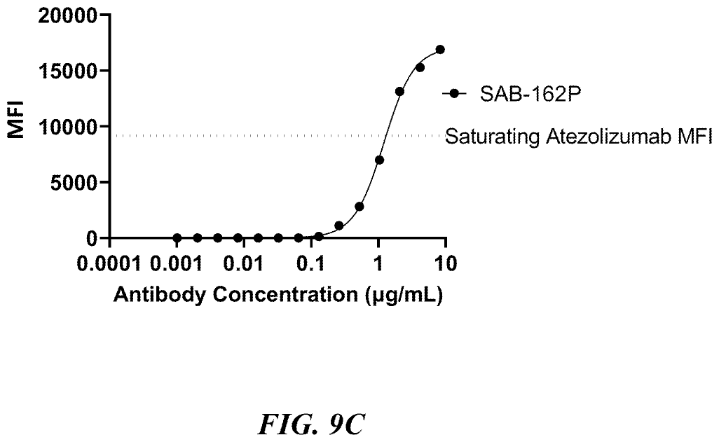

An SAB-162P titration assay was performed to determine the antibody concentration required for saturating binding to human NSCLC cells. For comparison, the saturating concentrations of atezolizumab to the same cell lines were measured. The primary antibodies, SAB-162P and atezolizumab, were directly conjugated to AF-488, and antibody serial dilutions were added to the cells. Saturating binding levels of SAB-162P and atezolizumab were determined for three NSCLC cell lines, HCC827 (FIG. 9A), H292 (FIG. 9B) and H1975 (FIG. 9C). Levels of SAB-162P continue to increase with increasing amount of added primary antibody. However, atezolizumab reached saturation levels at a lower MFI than SAB-162P. This difference may be due to the polyclonal nature of SAB-162P where multiple antibodies bind to a single cell. Due to technical limitations of direct labeling of the primary antibody, a plateau in the graph indicating saturation levels for SAB-162P was not reached in the cell lines. This data indicates that SAB-162P binds to PD-L1-expressing cells in a dose dependent manner.

Antibody-Dependent Cell-Mediated Cytotoxicity (ADCC) Activity of SAB-162P

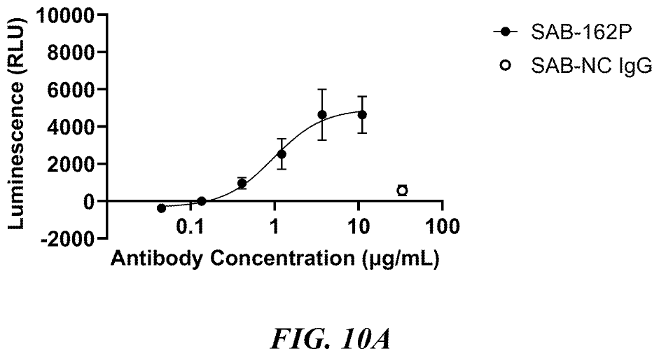

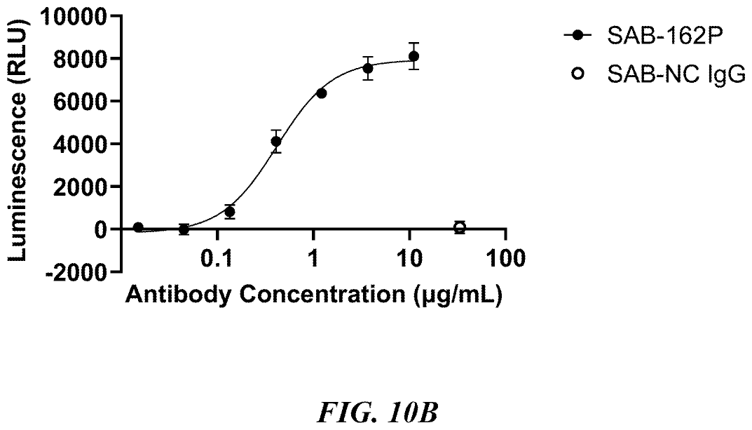

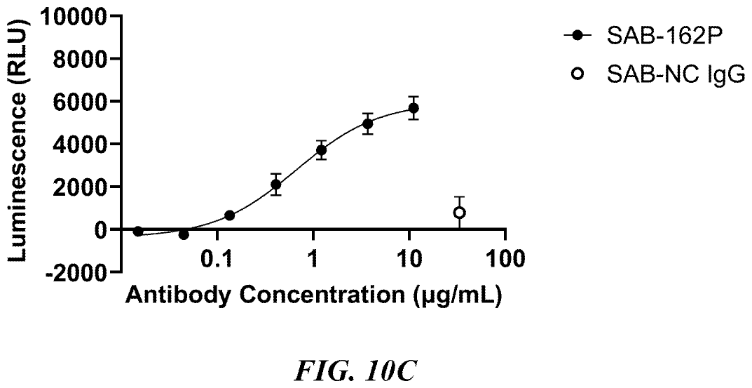

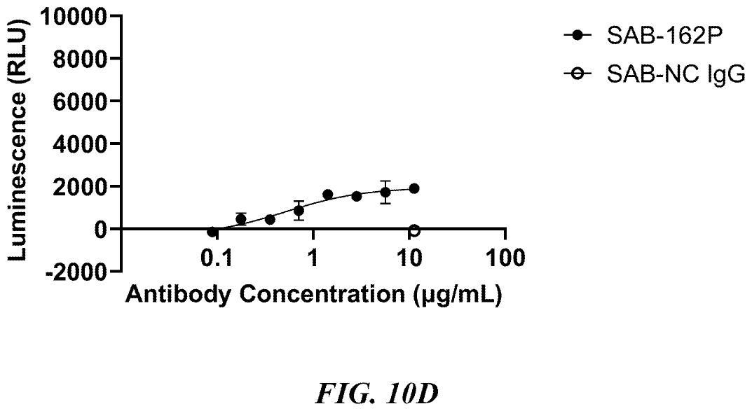

Antibody-dependent cell-mediated cytotoxicity (ADCC) is a mechanism by which antibodies target cancer cells for destruction by the cell-mediated immune system, such as natural killer (NK) cells. IgG antibodies bind to target antigens on cancer cells, and the Fc regions of the antibodies are recognized by FcγRIIIa (CD16) molecules on NK cells as well as macrophages. Upon binding of the IgG Fc to FcγRIIIa, a signal transduction cascade is initiated, and the nuclear factor of activated T cells (NFAT) mediates activation of cytokine genes. The InvivoGen Jurkat-Lucia™ NFAT-CD16 effector cells takes advantage of this NFAT pathway to create an ADCC reporter assay. Jurkat-Lucia™ NFAT-CD16 cells are human T lymphocyte cells that have been modified to stably express FcγRIIIa (CD16). Also, the cells stably express the Lucia luciferase reporter gene under the control of NFAT response elements. This assay gives a quantitative measurement of ADCC initiation by assessing cellular levels of luminescence. The human NSCLC cell lines H1975, H292 and HCC827 and H460 all have PD-L1 present on the cell surface (FIG. 8B) and were used as target cells for the assay. To determine the ability of SAB-162P to initiate ADCC, HCC827 (FIG. 10A), H460 (FIG. 10B), H1975 (FIG. 10C) and H292 (FIG. 10D) cells were incubated with serial dilutions of SAB-162P. Naïve TcB negative control pAb was added to the cells only at the highest concentration. The effector cells, Jurkat-Lucia™ NFAT-CD16 cells, were added to the NSCLC target cells in the ratio of effector cell to target cell (E:T ratio) of 6:1. The plates were incubated for 24 hours, and luminescence was measured. Compared to the negative control pAb, SAB-162P demonstrated ADCC killing of all 4 NSCLC cells (FIG. 10). The highest level of luciferase occurred at ˜10 mg/mL.

SAB-162P Blocks PD-1 Binding to PD-L1 on Surface of NSCLC Cells

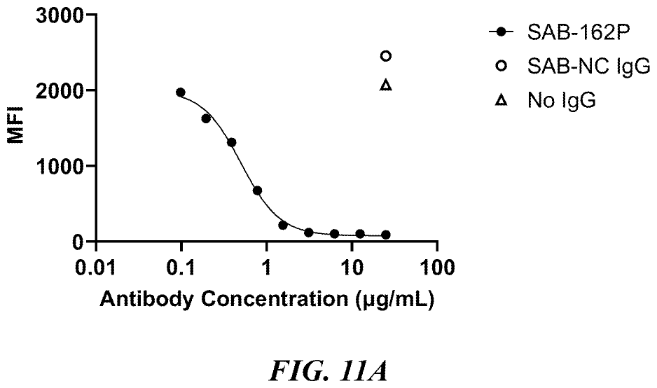

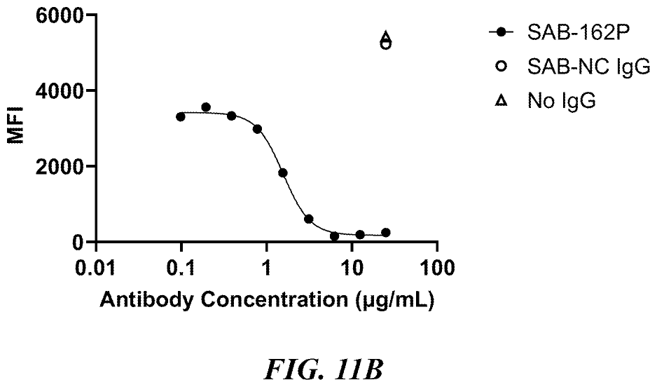

One mechanism by which antibody therapies increase the efficacy of cancer patients is to block ligand binding to the targeted receptor. By antibody blockade of PD-L1 from binding to PD-1, inhibitory signals to T cells are inhibited which enhances T cell activation leading to tumor regression. A binding assay using NSCLC cells was performed to determine whether SAB-162P blocks the binding of PD-1 to PD-L1. HCC827 and H1975 cells were pre-incubated with increasing amounts of SAB-162P. For controls, no antibody or SAB-NC IgG was added to the cells only at the highest concentration. Saturating levels of PE-labeled human PD-1-Fc-His protein were added, and the mean fluorescence intensity (MFI) was measured by flow cytometry. SAB-162P completely blocked binding of PD-1 to HCC827 (FIG. 11A) and H1975 (FIG. 11B). Maximum binding levels were demonstrated by adding saturating amounts of PD-1 to the cells with no antibody. Blockage of PD-1 to the PD-L1 was specific for SAB-162P as indicated by MFI levels of SAB-NC IgG being similar to the level of no antibody added.

SAB-162P Binding to PD-L1 Blocks PD-1 T Cell Inhibitory Signaling

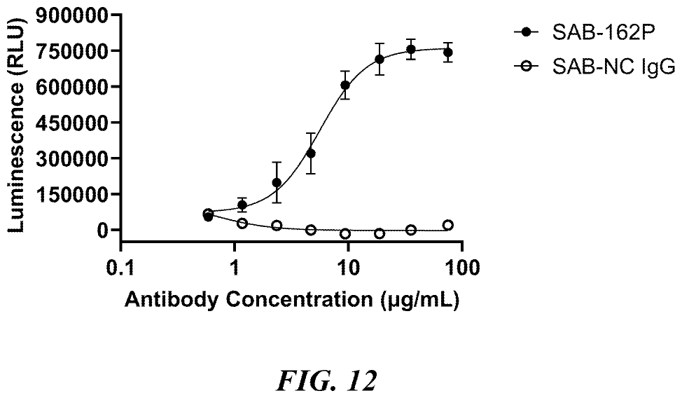

The ability of SAB-162P to block PD-1 induced T cell inhibitory signaling was evaluated in vitro via PD-L1/PD-1 Blockade Bioassay kit. For engineered effector cells, Jurkat T cells were altered to stably express human PD-1. In addition, the NFAT-induced luciferase reporter gene was stably transfected into the cell. CHO-K1 cells were modified to be target cells by stably expressing human PD-L1. A gene encoding a cell surface protein designed to activate cognate T cell receptors (TCR) in an antigen-independent manner was also stably transfected into the target CHO-K1 cells. When the Jurkat T cells and aAPC/CHO-K1 cells are co-cultured, the PD-1/PD-L1 interaction inhibits TCR signaling and consequently, NFAT-mediated luciferase activity is impeded. By blocking the interaction of PD-1 to PD-L1 with an antibody, the inhibitory signal is released resulting in TCR signaling which promotes NFAT-mediated luciferase activity. Increasing concentrations of SAB-162P or SAB-NC IgG were preincubated with the aAPC/CHO-K1 cells prior to the addition of Jurkat T cells. For SAB-NC IgG, the levels of luciferase were minimal for all tested antibody concentrations. However, increased luciferase expression directly correlated with increased SAB-162P concentration (FIG. 12). This result indicates that SAB-162P blocks PD-1 and PD-L1 interaction which releases PD-1 mediated T cell inhibition.

While embodiments of the present invention have been shown and described herein, those skilled in the art will understand that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### BUDAPEST TREATY DEPOSIT

Immunoglobulins described in this application were deposited with the American Type Culture Collection (ATCC®), located at 10801 University Blvd., Manassas, VA 20110, USA. The deposits were made under the terms of the Budapest Treaty on the International Recognition of the Deposit of Microorganisms for the Purposes of Patent Procedure on Nov. 2, 2021. The ATCC accession number for the aforementioned Budapest Treaty deposit is Deposit No. PTA-127159.

What is claimed is:

1. An ungulate-derived polyclonal human immunoglobulin composition, comprising a population of human immunoglobulins, wherein the population of human immunoglobulins specifically binds Programmed Death-Ligand 1 (PD-L1).

2. The composition of claim 1, wherein the composition is produced by immunizing a transgenic ungulate with an antigenic fragment of PD-L1.

3. The composition of claim 2, wherein the antigenic fragment of PD-L1 is a PD-L1 extracellular domain.

4. The composition of claim 3, wherein the antigenic fragment comprises, consists of, or consists essentially of SEQ ID NO: 15 or a variant thereof.

5. The composition of claim 3 or 4, wherein the antigenic fragment shares at least 80%, 90%, 95%, 96%, 97%, 98%, 99%, or 100% identity to SEQ ID NO: 15 or a fragment thereof.

6. The composition of any one of claims 1 to 5, wherein the population of human immunoglobulins binds MC38-hPD-L1 cells with at least as high affinity, or higher affinity than atezolizumab.

7. The composition of any one of claims 1 to 6, wherein the population of human immunoglobulins binds to a non-small cell lung cancer cell, optionally one or more of H292, H460, H1975, HCC827, and H1299 cells.

8. The composition of any one of claims 1 to 6, wherein the population of human immunoglobulins exhibit complement-dependent-cytotoxicity (CDC) activity, optionally at an IC₅ₒ of about 0.72 μg/mL or less.

9. The composition of any one of claims 1 to 6, wherein the population of human immunoglobulins inhibits tumor cell growth in vivo.

10. The composition of any one of claims 1 to 6, wherein the population of human immunoglobulins exhibit antibody-dependent cellular toxicity (ADCC) activity.

11. The composition of any one of claims 1 to 10, wherein the population of human immunoglobulins block PD-L1 from binding to the PD-1 receptor.

12. The composition of claim 11, wherein the population of human immunoglobulins blocks the PD-1 signaling pathway.

13. The composition of claim 12, wherein the population of human immunoglobulin enhances effector cell function, such as natural killer cells.

14. The composition of any one of claims 1 to 13, wherein the population of human immunoglobulins has an avidity for PD-L1 of at least 0.1 1/sec, at least 0.01 1/sec, at least 0.001 1/sec at least 0.0001 1/sec, or at least 0.00001 1/sec.

15. The composition of any one of claims 1 to 13, wherein the population of human immunoglobulins has an avidity for PD-L1 of 0.1 to 0.01 1/sec, 0.01 to 0.001 1/sec, 0.001 to 0.0001 1/sec, or 0.0001 to 0.00001 1/sec.

16. The composition of any one of claims 1 to 15, wherein, the population of human immunoglobulin composition is substantially similar to ATCC Deposit No. PTA-127159 or wherein population of human immunoglobulins has an avidity for PD-L1 at least 50%, at least 60%, at least 70%, at least 80%, at least 90%, at least 100%, at least 110%, or at least 120% as great as that of ATCC Deposit No. PTA-127159.

17. A method of making polyclonal human immunoglobulin specific for Programmed Death-Ligand 1 (PD-L1), comprising administering an antigenic fragment of PD-L1, or a polynucleotide encoding the antigenic fragment, to a transgenic ungulate,
wherein the transgenic ungulate comprises a genome comprising a human immunoglobulin locus or an artificial chromosome comprising a human immunoglobulin locus, and wherein the transgenic ungulate produces a population of human immunoglobulins that specifically binds PD-L1.

18. The method of claim 17, comprising administering the antigenic fragment or polynucleotide encoding the antigenic fragment 3, 4, 5, or more times.

19. The method of claim 17 or claim 18, comprising collecting serum or plasma from the transgenic ungulate.

20. The method of any one of claims 17 to 19, wherein the serum or plasma comprises a population of fully human immunoglobulins.

21. The method of any one of claims 17 to 20, wherein the antigenic fragment of PD-L1 is a PD-L1 extracellular domain.

22. The method of claim 21, wherein the antigenic fragment comprises, consists of, or consists essentially of SEQ ID NO: 15 or a variant thereof.

23. The method of claim 21 or 22, wherein the antigenic fragment shares at least 80%, 90%, 95%, 96%, 97%, 98%, 99%, or 100% identity to SEQ ID NO: 15 or a fragment thereof.

24. The method of any one of claims 17 to 23, wherein the population of human immunoglobulins binds MC38-hPD-L1 cells with at least as high affinity, or higher affinity than atezolizumab.

25. The method of any one of claims 17 to 24, wherein the population of human immunoglobulins binds to a non-small cell lung cancer cell, optionally one or more of H292, H460, H1975, HCC827, and H1299 cells.

26. The method of any one of claims 17 to 25, wherein the population of human immunoglobulins exhibit complement-dependent-cytotoxicity (CDC) activity, optionally at an IC₅ₒ of about 0.72 μg/mL or less.

27. The method of any one of claims 17 to 26, wherein the population of human immunoglobulins inhibits tumor cell growth in vivo.

28. The method of any one of claims 17 to 27, wherein the antigenic fragment is administering in a pharmaceutical composition comprising Montanide ISA-206 and/or Quil A.

29. The method of any one of claims 17 to 28, comprising:
a) administering a polynucleotide encoding the antigenic fragment of PD-L1;
b) administering a polynucleotide encoding the antigenic fragment of PD-L1, three to four weeks later;
c) administering the antigenic fragment of PD-L1, four weeks later
d) administering the antigenic fragment of PD-L1, four weeks later; and
e) administering the antigenic fragment of PD-L1, four weeks later.

30. The method of any one of claims 17 to 29, comprising purifying the human immunoglobulin to produce a composition according to any one of claims 1 to 16.

31. The method of any one of claims 17 to 30, wherein the population of human immunoglobulins exhibit antibody-dependent cellular toxicity (ADCC) activity.

32. The method of any one of claims 17 to 31, wherein the population of human immunoglobulins block PD-L1 from binding to PD-1.

33. The method of claim 32, wherein the population of human immunoglobulins blocks the PD-1 signaling pathway.

34. The method of claim 33, wherein the population of human immunoglobulin enhances effector cell function, optionally natural killer cells.

35. The method of any one of claims 17 to 34, wherein the population of human immunoglobulins has an avidity for PD-L1 of at least 0.1 1/sec, at least 0.01 1/sec, at least 0.001 1/sec at least 0.0001 1/sec, or at least 0.00001 1/sec.

36. The method of any one of claims 17 to 35, wherein the population of human immunoglobulins has an avidity for PD-L1 of 0.1 to 0.01 1/sec, 0.01 to 0.001 1/sec, 0.001 to 0.0001 1/sec, or 0.0001 to 0.00001 1/sec.

37. The method of any one of claims 17 to 36, wherein, the population of human immunoglobulin composition is substantially similar to ATCC Deposit No. PTA-127159 or wherein population of human immunoglobulins has an avidity for PD-L1 at least 50%, at least 60%, at least 70%, at least 80%, at least 90%, at least 100%, at least 110%, or at least 120% as great as that of ATCC Deposit No. PTA-127159.

38. A pharmaceutical composition, comprising the composition of any one of claims 1 to 16 and optionally one or more pharmaceutically acceptable excipients.

39. A method of treating or preventing cancer in a subject in need thereof, comprising administering an effective amount of the composition of any one of claims 1 to 16 or the pharmaceutical composition of claim 38 to the subject.

40. A rabbit immunoglobulin composition, comprising a population of rabbit immunoglobulins, wherein the population rabbit immunoglobulins specifically binds Programmed Death-Ligand 1 (PD-L1).

41. The composition of claim 40, wherein the composition is produced by immunizing a rabbit with an antigenic fragment of PD-L1.

42. The composition of claim 41, wherein the antigenic fragment of PD-L1 is a PD-L1 extracellular domain.

43. The composition of claim 42, wherein the antigenic fragment comprises, consists of, or consists essentially of SEQ ID NO: 15 or a variant thereof.

44. The composition of claim 42 or 43, wherein the antigenic fragment shares at least 80%, 90%, 95%, 96%, 97%, 98%, 99%, or 100% identity to SEQ ID NO: 15 or a fragment thereof.

45. The composition of any one of claims 40 to 44, wherein the population of rabbit immunoglobulins binds MC38-hPD-L1 cells with at least as high affinity, or higher affinity than atezolizumab.

46. The composition of any one of claims 40 to 45, wherein the population of rabbit immunoglobulins binds to a non-small cell lung cancer cell, optionally one or more of H292, H460, H1975, HCC827, and H1299 cells.

47. The composition of any one of claims 40 to 45, wherein the population of rabbit immunoglobulins exhibit complement-dependent-cytotoxicity (CDC) activity, optionally at an IC₅ₒ of about 0.72 μg/mL or less.

48. The composition of any one of claims 40 to 45, wherein the population of rabbit immunoglobulins inhibits tumor cell growth in vivo.

49. The composition of any one of claims 40 to 45, wherein the population of rabbit immunoglobulins exhibit antibody-dependent cellular toxicity (ADCC) activity.

50. The composition of any one of claims 40 to 49, wherein the population of rabbit immunoglobulins block PD-L1 from binding to the PD-1 receptor.

51. The composition of claim 40 to 50, wherein the population of rabbit immunoglobulins blocks the PD-1 signaling pathway.

52. The composition of claim 50, wherein the population of rabbit immunoglobulin enhances effector cell function, such as natural killer cells.

53. The composition of any one of claims 40 to 52, wherein the population of rabbit immunoglobulins has an avidity for PD-L1 of at least 0.1 1/sec, at least 0.01 1/sec, at least 0.001 1/sec at least 0.0001 1/sec, or at least 0.00001 1/sec.

54. The composition of any one of claims 40 to 52, wherein the population of rabbit immunoglobulins has an avidity for PD-L1 of 0.1 to 0.01 1/sec, 0.01 to 0.001 1/sec, or 0.0001 to 0.00001 I/sec.

55. The composition of any one of claims 40 to 54, wherein, the population of rabbit immunoglobulin composition is substantially similar to ATCC Deposit No. PTA-127159 or wherein population of rabbit immunoglobulins has an avidity for PD-L1 at least 50%, at least 60%, at least 70%, at least 80%, at least 90%, at least 100%, at least 110%, or at least 120% that of ATCC Deposit No. PTA-127159.

56. A method of making polyclonal rabbit immunoglobulin specific for Programmed Death-Ligand 1 (PD-L1), comprising administering an antigenic fragment of PD-L1, or a polynucleotide encoding the antigenic fragment, to a rabbit,
wherein the rabbit produces a population of rabbit immunoglobulins that specifically binds PD-L1.

57. The method of claim 56, comprising administering the antigenic fragment or polynucleotide encoding the antigenic fragment 3, 4, 5, or more times.

58. The method of claim 56 or claim 57, comprising collecting serum or plasma from the rabbit.

59. The method of any one of claims 56 to 58, wherein the serum or plasma comprises a population of fully rabbit immunoglobulins.

60. The method of any one of claims 56 to 59, wherein the antigenic fragment of PD-L1 is a PD-L1 extracellular domain.

61. The method of claim 60, wherein the antigenic fragment comprises, consists of, or consists essentially of SEQ ID NO: 15, or a variant thereof.

62. The method of claim 60 or claim 61, wherein the antigenic fragment shares at least 80%, 90%, 95%, 96%, 97%, 98%, 99%, or 100% identity to SEQ ID NO: 15 or a fragment thereof.

63. The method of any one of claims 56 to 62, wherein the population of rabbit immunoglobulins binds MC38-hPD-L1 cells with at least as high affinity, or higher affinity than atezolizumab.

64. The method of any one of claims 56 to 63, wherein the population of rabbit immunoglobulins binds to a non-small cell lung cancer cell, optionally one or more of H292, H460, H1975, HCC827, and H1299 cells.

65. The method of any one of claims 56 to 64, wherein the population of rabbit immunoglobulins exhibit complement-dependent-cytotoxicity (CDC) activity, optionally at an IC₅₀ of about 0.72 pg/mL or less.

66. The method of any one of claims 56 to 65, wherein the population of rabbit immunoglobulins inhibits tumor cell growth in vivo.

67. The method of any one of claims 56 to 66, wherein the antigenic fragment is administering in a pharmaceutical composition comprising Montanide ISA-206 and/or Quil A.

68. The method of any one of claims 56 to 67, comprising:
a) administering a polynucleotide encoding the antigenic fragment of PD-L1;
b) administering a polynucleotide encoding the antigenic fragment of PD-L1, three to four weeks later;
c) administering the antigenic fragment of PD-L1, four weeks later
d) administering the antigenic fragment of PD-L1, four weeks later; and
e) administering the antigenic fragment of PD-L1, four weeks later.

69. The method of any one of claims 56 to 68, comprising purifying the rabbit immunoglobulin to produce a composition according to any one of claims 1 to 16.

70. The method of any one of claims 56 to 69, wherein the population of rabbit immunoglobulins exhibit antibody-dependent cellular toxicity (ADCC) activity.

71. The method of any one of claims 56 to 70, wherein the population of rabbit immunoglobulins block PD-L1 from binding to PD-1.

72. The method of claim 71, wherein the population of rabbit immunoglobulins blocks the PD-1 signaling pathway.

73. The method of claim 72, wherein the population of rabbit immunoglobulin enhances effector cell function, optionally natural killer cells.

74. The method of any one of claims 56 to 73, wherein the population of rabbit immunoglobulins has an avidity for PD-L1 of at least 0.1 1/sec, at least 0.01 1/sec, at least 0.001 1/sec at least 0.0001 1/sec, or at least 0.00001 1/sec.

75. The method of any one of claims 56 to 73, wherein the population of rabbit immunoglobulins has an avidity for PD-L1 of 0.1 to 0.01 1/sec, 0.01 to 0.001 1/sec, 0.001 to 0.0001 1/sec, or 0.0001 to 0.00001 1/sec.

76. The method of any one of claims 56 to 73, wherein, the population of rabbit immunoglobulin composition is substantially similar to ATCC Deposit No. PTA-127159 or wherein population of rabbit immunoglobulins has an avidity for PD-L1 at least 50%, at least 60%, at least 70%, at least 80%, at least 90%, at least 100%, at least 110%, or at least 120% as great as that of ATCC Deposit No. PTA-127159.

77. A pharmaceutical composition, comprising the composition of any one of claims 40 to 55 and optionally one or more pharmaceutically acceptable excipients.

78. A method of treating or preventing cancer in a subject in need thereof, comprising administering an effective amount of the composition of any one of claims 40 to 55 or the pharmaceutical composition of claim 77 to the subject.


SEQUENCE LISTING (17 sequences)

SEQ ID NO: 1 - DNA, 6802 bp, Homo sapiens
cagcagggtcacaagggcaggccgggtccttgtggagagcacatttagtgggagggacatgatttcccttcaaagtgcccattctggacgcttcccgttccatgctggacgcttcctcttccacgctggatgcttcctgttccacactggatgcttcctgttccacgctggatgtttcctgttacactctggatgcttcctgttccacactggatgcttcctgttccatcctggatgcttcctgttccatgctggacatttcctgttccactctggatgctccctgttccatgctggatgcttcctgttccatgctggatgcttcctgttccatgctggacatttcctgttccactctgcatgcttcctgttccactctggatgcttcctgttccatggtggacgtttcctgttccactctgcatgcttcctgttccatgctggatgcttccttttccattctggatgcttcctgttccatgttggatgtttcttgttccactctggatgcttcctattccattctggatgcttcctgttccatgctggacatttcctgttccatgctggatgctttctgttacatcctggatgcttcctgttccatgctggatgttttttgtttgactctggatgcttccagttccattctggatgcttcctgttccatgctggatgcttccttttccattccgcacaattcctattccattctggacacttcctgtgcgacacctccttgggttttctgtctgcccagtccctctatcctcatcccgttccctgctacctcccacctccacaatcgtccttgcccagctcctccctctctctagagcttcggcctggcaaggtccctcctgatctcagtccaggctcccccagcacaggtaggagcctagcacctgcccttggacctccccaccctgcatggtgccagcatcccccggtccccagggaggccccatttctctctctgcttgtagtccagtggccctggagtcccactgcaactcgggtgtgcccctgacctctgaggaagttaagtgtcctgtccctagccaggctatcccgtctgctcagccccagggccctgcccccaaccccttcccctcacctgcaccacaggctctggccaactctgcccaggccctgaatgggcccctctggctcccctctgctgctacactgccctgcaccacctccactcagcttcagtgtgttcatccacctgtcccaagtcccctcggcccccaggagcacagctggtggccctggctcctggcagcccatcttgttccttctggagcaccagcctcagaggccttcctgtgcagggtccacttggccagccctgggaccctcctggtctcaagcacacacgttctccctgcagccagacctgcccctgcctgtgagctcagccctgagccttggaatgccttcccatctccatcccagctcgcctttgccagctgctcagcaggatgaactcacactcccctccctgcaccatgagtcagagccagctggagacacgcccaggccaaagcagccaccagggcctagtgggggccagaagcttcagatgagaggcccaggtattgagaggctgagatcacgggcagaatggtcataatcgctgccagtatcagtccagccccagggactcagagacagagaaaagagcagcacacaaggtctgggctccccaccttctcccgtgagtacgggggagtatgggggcagccaccacccccatccccacacacccatgaggcagcctcggctgtgtctggactccccctcgccctctgacccagaaaccaccagaagaaaagggaacttcaggaagtaagtggtgccgccggtttcaatcctgttcttagtctttgcagcgtggagttcacacacctggggacctgggggccgagctgtgatttcctaggaagacaaatagcggctgacggcaggggcggggctgcccacatgtacctcgccagaacaggaagggctgagacccccacctcggtgagtggggtcagcacagggcaggggcacaggctcgggaggaggacagagcctgggggcagccgtgggcgctcctggacctgagatgctgaacaggctccaagaggctggggagacatggggtcgaggccggccccacatggaggcccaagcggagccagcacgggggaggtgggcagccttcaggcaccgatgcccacccagtgcgagacgacggggaccgtgggcaggggcttccaagccaacagggcaggacacaccagaggctgactgaggcctccaggacgaccgggctgggagcacgaggaacatgactggatgcggcagagccggccgtggggtgatgccaggatgggcacgaccgacctgagctcaggaggcagcagagcgagggaggaggagaggccccaggtgaacggaggggcttgtccaggccggcagcatcaccggagcccagggcagggtcagcagtgctggccgtggggccctcctctcagccaggaccaaggacagcaggtgagccgggagcagagcagggagggtgagtgtggcagcaggacaggagggtggaagccaaggagcccagaggcagaggcagggacaggggaggcacaggggctgggctcagagccagctgatggggttggggcacctgctggcggggagcagggctgtggtcagcagtggagaggaggggagagctgtgctgagtgcacgggcgggagaagggaagagtccagggaggcccagaaaggcccagagtgcagcaggcctggggcgaggggaagggctgaggctccgtgcgttcagggaactgacccagcagagcagaggccactgaggagctgaggttccagagaggcttccagagcaggagcagtgcagggacaggaggatccgggagctcattcaggaggggcacatgggcaagggcaaggggctctgttggggagacctgactggacactggggctgctccacagcatagggaacacgccaagtgctgcaaaatcaaaaatgagggcagaaaaacagcccaaacctggacagagggtgccaggacaggcaggggggcaacagtgacctgagtgacattgctgcccgggttgagggagggcagagtgagcagggagcaggcattggagctcagggaccaggaccaagcagccacaggtgagcagggcaggtgggggcagaaggagcagggggcacctcctggagctcaggggaccagggcagagcagcctcaggtgagcaggggctggtgggcggcaggatgagcagggggaagaccctggagctcaggggaccagggcagagcatcagaaggtgagcatggctagtgggagatgggcaagcagggggcagcccctggagctcaggggaccaggacagagcatcaggaggtgagcatggctagtgggaggtgggcgagcagggggcagcccctggaactcaggggaccagggcagagcagccgcaggtgagcacgggctggtgggaggcgggaggaacagggggcagctcctggacttcaggggaccagggagggcatctgaaggtgaacaggggccagtggggggcaggatgagcagggggaagctcctggagctcagggagccaaggcagagcagccgcaggtcagcaggggcaggtgggaagcatggggagcaggtgggcagcccctggagctcagagagccagggcagatcatccacaggagagcaggggctggtaggaagcaggaggagcaagtgggcagcttttggagctcagagcaccagggcagaagagcctcaggtgagaaggggcaggtgggaggcagaataagcaggggacagcccctggacctcaggagaccagggcagagcatcacaacgtcagcatggctggtgggaggtgggcgagcagggggcagcccctggacctcagagagccagggcagatctgcaggtgagcaggggcaggtgggaggcaggaagagcaggaggcagctcctggagctcaggggatcagggcagagcagccacaggtgagcaggggcaggtaggaagcagaaagatcaggggtcagcccctggagctcaggggacaaggggagagcatcagaaggtgagcaggactgaggctcagcctcagggagccagggcagagcagctgcaggtgagcagggccggtgggaagcaggaggagcaggtgggcagcccctggagctcagagagccagggaagatcatccgcaggtgagcaggggctggtgggaagcaggaggagcaaggggcagctcctggagctcaggggaccagggcagagcagtcgcaggtgaacaggggcaggtggggggcaggaggagcaaggagcagctcctggagctcaggggaccagggcagagcagtcgcaggtgaacaggggcaggaggagcaaggggcagctcctggagctcaggggaccagggcagagcagccgcaggtgagcaggtgcaggtggggggcaggaggagcagggggcagctcctggagctcaggggaccagggcagagcagccgcaggtgagcaggggcaggtggggtgcaggaggagcagggggcaggcactggagctcaggggaccagggcagagcagtcgcaggtgaacaggggcaggtggggggcaggagtagcaaggggcagctcctggagctcaggggaccagggcagagcagtcgcaggtgaacaggggcaggtggggggcaggaggagcagggggcagctcctggagctcaggggaccagggcagagcagccgcaggtgagcaggtgcaggtggggggcaggaggagcaggggtcaggcactggagctcaggggaccagggcagagcagccgcaggtgagcaggggcaggtggggggcaggaggagcagggggcaggcactggagctcaggggaccagggcagagcagccgcaggtgagcaggggcaggtggggggcaggaggagcagggggcaggcactggagctcaggggaccagggcagagcagccgcaggtgagcaggggcaggtggggggcaggaggagcagggggcaggcactggagctcaggggaccagggcagagcagccgcaggtcagcagggccggtgggaggcaggacgagcaggggacaggcactagagctcagggcaaggcagccacaggtgagcagggctggtgggaggcatcactcagctcctagactttggcaggagctgggtagttgctggcaacagacagctgagggctggtgaaagtgcagtgcagcctcctggtgccgggaagggagtgtgagtccatcccactgagcagttggcaagggcgagctgggatggagaagggaaggcgttccagggctcagggctgagctctcaggcaggggcaggtgtggctgcagggggaacgtgtgcttgagaccaggagggtcccacggctggtcccagcggaccctgggcaggaaggcctctgaggctggcgccccagaaggagcaagatgggctgccaggagccagggccaccagcacaatgaagctgagtggaggtggtgcagggcagtgtagcagcagagggcagccagaggggcccattcagggcctgggcagagtcagccagagcctgtggtgcaggtgaggggaaggggtggtgagcggggccctggggctgagcagaggggatggcctggctgagggcagggcgcttagcctcctcagaggtcaggggcacaccccacctgcagtgggactccagggccactgggccagcggcagagagaaatggggcctccctgtggcctgggggtcctggcaccatgcagggtggggagggccaagggcaggtgcaaggctcctacctgtgctggggggcctgggttgagcccagcagggaccttgccgggggaagctctggagagagggaggaggtgggctggtggccgagaaggccaggccagggctgggagggtgacggtgtggtgactgagcctccagaagtaatgcaggacactgggaggcagggggcatccaggcactcagggccctgacctgggctgctgcacactggggctaaggggaaaggaggggagaggctgaggaggaggctccaggaggctattccaaggcagggggttccggggccctggggctgaagggcgccgaccctatgcagtgtctggcccctctgctgcacagaagaaaagggccttggagggcagagggcaggctatgaccagggccctgggcaagtcaggccaactcactaggggagggccacgctggggcggcagggtcaggggcttcagggggctcgggggacccacgagaagccatctgagaacagtgtccactggtcaagccaggcacccataaaaggctggagtggggccaatgggcatgagccgtccctgaggtggcaccgatggccagagctgaggccaagctagaggccctggactgtgctgactcccggcagacacagagcgctgacctggctgccgagccccgcctcctaggctgcaggggtgcctgcagaagggcaccacagggccaccggtcctgcaagctttctggggcaggccgggcctgaccttggctttggggcagggggtgggctaaggtgacgcaggtggcgccagccaggcgcacacccaatgcccgtgagcccagacactggacgctgaacctcgcggacagttaagaacccaggggcctctgcgccctgggcccagctctgtcccacaccgcggtcacatggcaccacctctcttgcag

SEQ ID NO: 2 - DNA, 9483 bp, Bos taurus
gatcaaacactgtcaaaccaacacaagagagccccagtaccagatgtgaggctccccccagaccccagtcacagtgcagcctgtagagggaacaggcccagtcgggagaccaggaccgagaaagtgacccctgtcagtcagtccagctactcagtcatgtccgactctttgcaaccccacagactgcagcacgccaggcctccctgcccatcaccaactcccggagcttgctcaaactcaagtccattgagtcggcgatgccatccagccatctcatcctctgtcgtccccttctcctcctgcccccaatccctcccagcatcagagtcttttccaatgagtcaactcttcgcatgaggtggccaaagtattggagtttcagcttcaacgtcagtccttccaatgaacacccaggactgatctcctttacaatggactgattggatctcattgtagtccaagggattctcaagagtcttttccaacagcacagttcaaaagcattgattttttactcagctttctttatggtccaactctcacatccatacatgactactggaaaaaccatagctttgactagacggacctttgtcagcaaagtaatatctctgctttctaatgtgctgtctacgttggtcatagcttttcttccaaggaacaagtgtcttttaatttcatggctgcagtcaccatctggagtgattttggagcccaagaaaatagtctgtcactgtttccattgtttcctcgtctattttgccatgaatgccatgatcttcatttttcgaatgttgagttttaagccagctttttcactctcctctttcactttcatcaagagtctctctagttcctcttcattttctgccatgagagtggtgtcatctgcatatctgaggttattgctatttctcctggtaatcttgattccagcttgtgcttcctccatccaggtatgtcacatgatgtactctgcatagaagttaaataagcagggtcacaaaatacaatcttgatgttctccttttccaaattttaaccagtccgttgttccttatctggttctgttacttcttgacctgcatataggtttctcaggaggcagttaatgtggtctggtattcccctctctttaagaatgttccagtctgttgtgatccacccattcaaaggctttagtgtagtcaataaagcagaagcagtaaaatgctgccactttctaggtggagctcacatatggataccacacccaaaccttgaccaagccagaacctcatgacaatcatgtgcagtgctataaatgatctcagctgttacacagtcactatttacaaaatgtggggcaagagagcctgaacatgccatcttggccaattttctctctcctcatgggttgtttagctttttttctttttttttttttttaattaagctgtatgagttgcttgcacattttggaagtttattctttagtggtcacgtgatttgcaaatatcttctctaattctgcaggtagtcttgttttgttgatggttttccatggtgtacaaaagccttttagcttaattaggtcccatttgtttatttttctttttatttctattactctaggagatggatggagaaggaaatggcagcccactccagtgttcttgcctggagaatcccagggacgggggagcctggtgggctgccgtctatggggttgcactgagtcggacatgactgaagtgatatagcattagcattaggagataaatacaaaaagacaccaccttgatttatgtcaaagagtgttctgcccatgtttccctctgttttatagcctcccatttcacacttttgtctttaatccattttgagtttatttttgcccatggcactagagaatttataatttcatttattaacaggtagcaatccagtttttccagaacttattcaagagactgtcttttctccatcttacactcttgcctcctgggtcatacatgaattgatcataggtgggtgggattgttcctgggctgtctatcctgtctcactgatcactgatctataattgtggttttgtgccagcaccatagtgttttgatggccgtagctttgcagtatgctctgaagtctgggagcctgatgcctccagctccacttttgtttcttgagattgctttgactatttggggtctttatgtctcccaacaaattttcaaaagttttgttctgggtctgtggaaattgccattggtcatttgataggtgttacactgaacctgtagattgctttcggtagaagtgtcattggcacaatattgattcttctgatttaagagtatcttttctaaatagacatttctccaaaaaagacatacagatggctaacaaacacatgaaaagatgctcaacatcactcattatcagagaaatgcaaatcaaaaccactatgaggtaccatttcacaccagtcagaatggctgcgatccaaaagtctacaagcaataaatgctggagagggtgtggagaaaagggaaccctcttacactgttggtgggaatgcaaactagtacagccactatggagaacagtgtggagattccttaaaaaactggaaatagaactgccttatgatccagcaaccccactgctgggcatacacactgaggaaaccagaagggaaagagacacgtgtaccccaatgttcatcgcagcactgtttataatagccaagacatggaagcaacctagatgtccatcagcagatgaatggataagaaagctgtggtacatatacacaatggagtattactcagccattaaaaagaatacatttgaatcagttctaatgaggtggatgaaactggagcctattatacagagtgaagtaagccagaaggaaaaacataaatacagtatactaacgcatatatatggaatttagaaagatggtaacaataacccggtgtacgagacagcaaaagagacactgatgtatagaacagtcttatggactctgtgggagagggagagggtgggaagatttgggagaatggcaatgaaacatgtaaaatatcatgtaggaaacgagttgccagtccaggttcgatgcacgatgctggatgcttggggctggtgcactgggacggcccagagggatggtatggggagggaggagggaggagggttcgggatggggaacacatgtatacctgtggtggattcattttgatatttggcaaaactaatacaattatgtaaagtttaaaaataaaataaaattggaagaataaaaaaaaaaaaagagtatcttttcatctgtttgtgtcaccttaatttctttcatcagtgtcttagagctttcagagcagaggaattttgcctcctagggaaggtttattcctaggtattttattctttttgatgggatggtaagtgggattctttccttaaattctctttatggtatttcattgctgccatacagaatgcaacagatttttgtgtattaattttgtatccataaattttatccaattcattcctgagctctagggttttctgtaccttctttagcattttgcatgtgttaaatcatgtcacctgcaaacagcaggtgtttcttacttggcttctggattccctgtgtccctttctctcccctgattcttacacttaggccttcactgtgctgcactgtgggcccttgctggttacctactatatacgcagtcgtgtggatgtattagcaccaaactcctaatttatccctgggatgtggtgggtcaaccaggcgcagtgcatgaggaagaggaacaccgtgacggctaggattcccactcctcccctgggtgcaagggcgccgcttaaaccaccccaccaacccacaagacatacctcagtcacatctggactcccccaggccctctgagacagaaaacactcagaagaaaagggaacttcaggaagcaagtcgcgtcaccaggtttcattcctgttcttagtcttcacagcactgggggaagggccctcacacctcctgggactggtgaccaagtcccagggagcagggctgcagacacagaacacagggaacttcaggaggcagatggagtcaccaggtttcattcctgttcttagtcttcacagcactgggggaagggccctcacgcctcctgggactggtgaccaagtcttagggagcagggctgcagaaacagcacgcagatcccaggagccacatgtgtctgtgggccagagcagggaaggggcccggagccccaggctgtggggcagccccctccatggcccccaccatgggtgagtggggaccccgaggatgagcgcacagggacaggaatgagagccctggagggagaccggcctcgggacgcccagaggggctggagcagccgagtgggcccaggggaggcagctgcccctggtcacagtgcaggacgagcgtgtggacagaacccagacagggtgcaccacaccaagagcactgctgcccagggctggagagctcagggggctgtgggccgtggaggagcaggatgggcttgggggcgggagggaagggatgtgcatgaccaggggctctggaggccggggctgggcagtcatgaggaccagccaggacggaaagagggtctgacctgaggacgcagggaagggagaggaccccagagcagtgaagtcttctggacaggccagtggagccagcgtcggtcccagggcagggccacatgcagggactgtgggtcccgactgggctggggccgagggcagcggcagacttgggggcaaggctgcagggccagccgggttctatgtccacagcaggcaccacggggctgaacccacagccccccaggacacagggggctgaccagagctcagggcctgaggaccagcagtgggagccagagagaggcaggtgacccgaaaactggaggcagcaaggggccagaggagggtgtggatggagggatagagaagagcagagagcaaaggcaggacctctgccaggaggggcccgggtggacagagctcctccaggggagagttgcctggaggccagtgggcacctggcaagtccagggatgagagctgaacccaggaaataaaggagtgaggagtggagatgggcaggggtgagggcttccagaggggccagaggggcaagaaaggctgagggggcggcccaaggacagggggcagcaaggctgcaggtgagcaggggctgggagggcaggggggcccctagagatctgggggagcaggcacagcttgtgggtgaaaaggggttaggagggcagggggcccctggtgtttgggggagcagatagagcttttgtaggtgagcagggagctgggagggcgggggagagggtggcctggtgctttggggagcaggtacagcctgtgagggtgagcaggggctgggagggcagggggtgttcctggtgtttggggtagaaggcacagcttatgggtgagcaggggctgggagggcagggggagcccccggtgctttgggggagcaggtactggttatgagggtgagcaagggctgggaggacggggggagcccccagtgctttgggggaacaggtacaggttatgagggttgagcagggggctgagagtaaggaggtgagcctagtgctttgggggagcatgtacagcctgtgaaggtgagcaagggctaggaggacagtgggatcccctggtgctttaggggagcaggtacagcttgtgagggtgagcaagggactgggaggacaaggggtgcccctggtgtttgggggagcaggtactagttatgagggtgagcaggggctgggaggactgggggagcccctagtgctttgggggagcaggtacaggttatgagggtgagcaggggctgggaggacagggggtgttcctggtgtttggggtagaaggcacagcttatgggtgagcaggggctgggaggacaggggggcccctggagatatggggaagcaggtacagcttgttggtgagcaggggctgggaggacagggggagcccctggtgctttgggggaacaggtacaggttatgagggtgagcagggggctgagagtaaggaggtgagcctagtgctttgggggagcatgtacagcctgtgaaggtgagcaagggctaggaggacagtgggatcccctggtgctttgggggagcaggtacaggttatgagggtgagcaaggggctgggaggacaaggggtgcccctggtgctttgggggaacaagtacagcctgtgacggtgagcagggagctgggagggcagggggtgcctctggtgctttgggggagcaggtacagcttgtgagtgtgagcaagggctgggagaactgggggtgtccctggtatttgggggagcaggtactggttatgagggtgagcaggggctgggaggactgggggagcccccagtgctttgggggagcaggtacaagttatgagggtgagcagggggcttagagtaagggggggtgcacctggtgctttggggagcgttacagcctctgagggtgagcatgggctgggaggacagtgggatcccctggtgctttgggggagcaggtacagcctgtgagggtgagcaggggctgggaggacagggggagcccttggtgtttgggggagcgtgtacagcctgtgagggtgagcaggggctgggaggaaagtgggagcccctggtgtttgggggagcaggtacagcctgtgagggtgagcaggggctgggaggaaagtgggagcccctggtgtttgggggagcgtgtacagcctgtgagggtgagcaggggctgggaggaaagtaggagcccctggtgtttgggggagcgtgtacagcctgtgagggtgagcacgggctgggagggcagggggagcccctggtgctttgggggagcaggtacaggttatgagggtgagcaaggggctgggagggcagggggagcccctggtgtttgggggagcatgtacagcctgtgagggtgagcaggggctgggagggcagggggcgcccctggtgttttggggagcggtacagcttgagagggtgaacaggggctgggaggacagggggagcccctgatgctttgggggagcaggtacagcttgtgagtgtgagcaagggctgggaaaactgggggtgtccctggtgtttgggggagcaggtactggttatgagggtgagcaggggctgggaggactgggggagcccgtggtgctttgggggagcaggtacaggttatgagggtgagcagggggctgagagaaaggggggtgtgcctggtgctttggggagcattacaatctgtgagggtgagcaggggctgggagggcagggggagcccctggtgctttgggagaacaggtacagcctgtgagggtgagcaggggctgggaggacagggggagcccctggtgtttgggggagtgtgtacagcctgtgagggtgagcaggagctgggagggcagggggagcccctggtgctttgggggagcaggtacaggttatgagggtgagcaggggctgggaggactgggggtgtgcctgatgctttgggggagcaggtacagcttgtgagggtgagcagggggccgggaaagcagggtgtatctcaaaaactttaggggatcaggtagtgcttctggggatgagcagaggtctgggaggacagggggtgcccttggagttggagagcaggtacagtctgtgaggtgagtagagggctgggcaggcacagggagcccctggtgctttgggggaacaggtgcaggtcgtgagggtgagcaggggctgggaggacagggagtgtccctggtgtttggggtagaaggcacagcttatgggtgagcagggcctgggaggacagggggagcccctgttgctttgggggagcaggtacagcttgtgagggtgagctgggtctgggaagacagggggagcccctggtgctttgggggagcaggtacagcttgtgagggtgagctgggtctgggaagacagggggagcccctggtgtttgggagagcagatacagcctgtgagggttagcaggggctgggaggacagggggtgcccttggagttggagagcaggtacagtctgtgaggtgagtagagggctgggtgggcagggggagcccctggtgctttgggggaacaggtaaaggttatgagggtgagcagggggctgagagtaagggcgtgtgcctgttgctttggggaagcatgtacagcctgtgagggtgagcaggagctgggagggcaggggggagcccctggtgctttgggggagcaggtacagcctgtgagggtatccagggggctgggaggacatggggtgcccctggagatttttggaagcagtaatagcttgtgggtgagcaggaagctgcgaggacaggaggtgcccctgttgctttgggtgaacaggtacaacttgggtgagcaggggctgggaggacagggagtgcccttggagatttgggggagcaggtgcagcttctggggataagcagggggctgggagttcagggtattaggggagcagacaaagatcgcgcagctgagcaggcgctggaatgtcacgcccctgccgacgacctcagttgaccatgtgtgtgctgagcacatcggtacgaaagggtccgagggtgcaaggggccatttgtgctgtgggccagatgcagggacacagggcagtgtgagccctgcagagaagatggggcccaagagcgcagctgtccagagctgagtccagagggctgagaccagtggtgtgggtgctggacgagggtgaagggctggcgtgagggagcaagggcacagggctgggagagctcaggccacagctggtgaagcagagggctgagacaaggggtgtaggccacccctcaagacaatgggggtgctgtttggtggagggtctgcatgaagaacaaagcaaggaggagcgaagacccgagaggaagggctctggggctgcagggaaggggcgccctgagggcaccgggtgggctgcacttctgggccgagggtctgcctgtcccagcagcctgtgtggctcaggagccaaagtggggccagacctgggacgctgtgcttgatagggcggtgcaaggggccaggagctacagggcgcccactggcagtcagtcgggctgcaggcccagggagctgaccaggctgggggagcaggggccatgggaagggccaggcgcccacaggagggaaggggccctgggctctgatccaggaggccacactcaccgacccaagctgtgtccagacaccccaactgcagtacagagagccgggtggccaccatgccggccggtcatcagaccctggaagcaggtggtggctgggctcggaggtgccccaggcctgggcacctgaggtcctgctggaccccgcattcacccagcctcctctctcacag

SEQ ID NO: 3 - DNA, 10298 bp, Equus ferus
  misc_feature (6918)..(8037): n is any nucleotide
agatatcccccaagtcatgtcacaatactaccatgttcaaagtacatgaatgctttgaaacagactggtctcagtggctgggtgttccttgggtggctcctaatggaactcagtggttgtgtggaacaaacctctagtcttggctcctgcctggatggctgggacactgcaccctggacttcctttggaggcagggaaggagtcgccctgagctaacagctggtctcaatgttcctcccagtaaggccagatggactccatcagtgttcactggtatgaccaccagcggctatatttgctgcttccttgggctacaagatgtcatatcacatagaggctctaactaaatttactcaaactgccctcaatgaaagtcaggtaggaatacccttattaaatactcaaatgtctttagtggggagggctgtctccccatcagaatggccttagatagtctaacagcatcccaaggtgggacatgtgcactcattcaaactgaacgctgcgtttttagacctgatgagtcttccaatgtgtcctctctgctaaagcacttggaaaagcaggtaaatgccttaagcgatcctgcatccaggcttgatttgcgtggttggctcccttcaggtgtggctgccctcttgaaatctgaattgcaattcctgtgtctgttactccttggaattcttctattaatcataatatgcaaactaattactctttcctttactcagtgttgtaagactggcatgcaggctagaataatggttgctcagccacttgaggtgattgattgatcttacaaccctggatgaatttccttctcataagactatgcctaagaactcatttttcaaataatcctttcaagttttgaattatcagcactgttagtgtagctgttctataattaggcacatgcagcatcatgagtcagatcaaagcataggtacaacgtttgtgtgacaatctaaagttactgaaaacttacataacctatggaatgcttcccctgtaattgtatacctctatgctcatccactatatccaactatttcctgccattgggcataactgagcacgtcaatgagataaaagcccagcactgatggccatcatggaccagggcaggcaacaatcaccatagcaccaagggacaatatttagatcatcagtgctttctataaaaacattatcgatcaaaggtgggaaaataatgaatcaatgaaagcaataagaaaagttaaaatgttgaggtatatgaggaagccatttggtttagactaattaggcctcatctagtttttctggaaagtcctgatgtgcctgtcgaatatgcattgtacatctgctgcatttacaatgtcccaaagcaagaatgatgcccttgaaattatcctgcccccttttggcatttctttatagatgagcacttcttcccagaatgcaaggattcgttactgacctactgtgtcatcttgtgatcactcacccgcaatactagcaaagcatcttgtgactgtagtaaaagagatactcctgtcatatgtgatgtacgtcccttgttccaagacggtatataaccacgctgcacacccggcttcttcacaacacttccttccttggtgaaggttattgtcccgggctatgtagtcctcaaattggctcaaataataaactcaccccaattttgattaacagattgattatagtttattgcctcaacatttcctgaccagaaattacatagatgaacgtgcttcttatgtaacggcagcgatgctcacagcctgcggagcacatgactgagggaggctgccttccgtgggagagatggcaaaactgcgtgggcgccatggagaggaggccgggccagagccgccgggagtgtgcagtggattatcatgaattcatcccggagggaccagagggcactcccttcaccaaggctgctgctgacggcgttttctgggtgtccacagcaacattcccaggaccacaggctcctccatcacctggtcctgccccatgcagaggttcacttccctcaccagaccccagggtacccatgcgactgactccatggggacagggccactgagggaccactgctgggtctctgagagcaggacctaaggagacagggctcctcctgggagtcaggcccgggcaccactcagcgtgggcaatccccatggaaagatcacagagacagagggaacacgggcgcccgcccagcccaggcgccagactccaggtggagccttccagcccctcgctgatgcgagtggagcagagaggagcgcccccgcccacagaagccggcactgcctgcaggccaccaacgcagaagcaaatcagcagaagagctgcagaggccactctctgtggggcggtccacagcacacccatgaaccgccggacacgatatgggacccgcacgtgggcgctgctctgagaaacctcaactctagaaagtaagcacgaggaccagagagcaggggaggcagagggacccagagtagaccgttgggggatgtggagtggctctggggacacccgaggccaaagggtgctgcaaatagcaacaggtgggagggccgactcccctcgtacatctcaggtcacctcctagaaggttcgctttggactttgctcttggcccacctaagcggccacagcgtacgcccctgcccaccaaccattggaaggagccaggcctgccttcacgtccagtcacctacctggcccccggcaattccagggtggggaccatggtgccgcccagggatccccgatccaaggcctaacgtgcaagagacgggatggcccaggtttgtacacggggccttgggtgtgccgaggcactgcaagtcatgagacactgagggtccgtgtccccccacagacaaaaagctgggtcctggcccacacacgggaggacaggaacagcatgctgtctctcagacagaggagacctttgggccccagtgaccaccgtggactctgtccttgtacatagacttctttctgtccccagaggacagctgtggatgggagaatatcttccctgggaccctgggtgctaccacccttcagtcagagatccagccatggatggagccagagagggatggagggaagaggcagggacccagaggaagacggctttgtacttagggggctggcctggcaggaggacaggatgaggccctgggctgagctgcgggctgtgagcaggacagcctgtgtccacaatggacgctgactagggcagggggaggtgtcctctgggctgtgggtaccagagggctcaggtgagaggcctgggtgccctggcctttggtggggccatcgggcataatccttgtgggagggagagagcaaagagaggcatgtgagcccggacttcctgcactctccccagagtcctgggcgccagaccccctccactcccacacacccacgtgtggcagcctctgcctcacgtctgtactccccccggctctctaagacagaaaccacccagaagaaaagggaacttcagggagcaagcggtgcccctgctttcagtcccgttcttagtctttgcagggtcgtggagagtgggttcccacctccgggaaccagctaccacctccgggaaccagctaccaactcctaggaagcagggcagagggcaacaagcccggggccgcgaggagccacatgtgcccggggccagagcaggggcgtggggggagctcctcggaagccctggcttccaccacctccacagcccccacccttggtgagtggggtccccacggagcagggggaagggagtggagtcagggcagtggaaacggttgcagagctacggggagcccaggcacccagggacgactccgaggggctcaagcagctgagccagcacgggggagggcaggctgcccacgagccacggtgccaaccggacgcaggcaccaggcagccatgggcagggcacgcccacgccaacagagctggacacaccagaggcccatgaaagttaaccttgaggaaattgatgaccttgaggccagaagcctgagggctgcatttggccagctgtgctgagggcaggaccagccaggagctgagctgctgcagcacggacgccaggatgcacagatgggtttgagcacagagagggggctggctcagagcaggggaggaagaggacctccaggtggcctaagggtcctgtccacgccaatgagccagctcaagccccagggcagggtcagaggtggccgccatggtgtcctaggctgtagccaggaccgagggcagcaggtgagccaggtgcagagcccaaagggccggcggatgcacccatgtgctcagaactcagactgcacagggctgggcaggagctcagtggctacaggcagacgttgggagccaggcagtgagggtccagagcaggtgctggggttgcagtgttgggagctgggtggcaaggggagctgacccagcaatggagggagcacagtcccgaggggccttccagatgggcttctgagcgccgtgaggggcacagagacggtgaagcccagctgggccaggagctcctccaggaggagaccacggcaggcaggggctctgccaggggagagttagccagagagagggtgggcatctggtggctccaggaggcaaaggccagtgatgggaaaggaatgagcccaggatggagggtgaacctgggctgagggtgctgggagaggcaggaggggcaaaggcagccccgggcagacccagcagcctggggctgagcttagggacctgagctgtgtggaggggatcgtaggacaggagggacagagggctgctgtggacctggagttccagggacaacgggaagaacagctacggatgagcaggggaaggtgggagggcacaaaaggacaggagcctacaccaagagatgcatgggggcaggcagagcagctacaggtgagcagaggccagtgggagggctggagggaaagggggctgcccaggggctctggaagggggcaggtagagcagctacaggtgagcagaggaatgggagaccaggagggacaggtcacagcccaggagccccatgggtgtggagagcagagcttggcatgtaagtaggaagcaggaagggctgaaggggcaggggctacccagcagctccacaggagcaggcagagaagctacaggtgagcaggggccggtgggagggctgcaggggcagggggctgcccaggagctccggggagcaggcagagcagctacaggtgagcaggggccgatgggagggctgcaggggcagggggctgcccaggagctccggggagcaggcagagcagctacaggtgagcaggggccgatgggagggctgcagggacctgcccaggagctctggcggagtagtcagagcagctacaggtgagcaggggccagtgggagggctggaggggcagggtcctgcccaggagttccaggggagcaggcagagcagctataggtgagcagacgctggtgagaggtctggaggggcagggggctgcccaggagctctagcgggagcaggcagagaagctataggtgagcaggggccggtgggagggctgcaggggccggggcctgccgaggagctccggcggggtagtcagggcagctataggtgagcaggggccggtgggagggctggaggggcagtgggatacccaggagctccgccagggtagtcagagcagctacaggtgagcaggggcaagcaggagggctggaggggcaggaggctgcccaggagctccggcggggtagtcagagcagctacaggtgagcaggtgccagcgggagggctggaggggcagggggctgcccaggagctctgggaggagcaggctgagaagctgtagttgagcaggggccagtgggagggctggaggggcaggcggctgcccaggagctccgggggagcaagcagagcagctataggtgagcaggggcaggtgggggggctggaggggcagggcaatgcccaggagctccggcggggtagtcagagcagctacagtggagcaggtgccagcgggagggctggaggggcagggggctgcccaggagctccgggggagcaggcagagcagctacaggtgagcaggagcaagtgggagggctggaggggcagaggactgctcaggagctccgggggagcaggcagagcagctacaggtgagcaggagcaagtgggagggctggaggggcagaggactgctcaggagctccaggggagcaggcagaacagctactggtgagcaggggtcagcgggagcgctggaggggcagggggctgcccaggagctccgggggagcaggcagagcagctataggtgagcaggggcaggtgggagggctggaggggcagggcaatgcccaggagctccggcggggtagtcagagcagctacagtggagcaggtgccagcgggagggctggaggggcagggggctgcccaggagctccgggggagcaggcagagcagctacaggtgagcaggggccggtgggagggctgcaggggcagggtcctgcccaggagctccacaggagcagacagagaagctataggtaagcagggccggtgggagagctggaggggcagggtccggcccaggagttccgggggagcaggcagagcaggtacaggtgagcaggcgctggtgagaggtctggagcggcaggtggctgcccatgagctctggggggagcaggcagagcagctataggttagcagtggccagcaggagggctggaggggcagggagcagttcaaggagctccagcggaataatcagagcagctataggtgagcatgggccagcgggagggactgggacggggcagggagctgccaggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngttccagggaatcttctacagggagctagatagagaaagtttataggtaaagcaggtgccggtcgggagaagttgaaggggcagggtcctgccccaggaagttcccggggggagccaggcagagcaggttacaggtgagcaggcgattgtgagaagtctggaagcggcaggtgtcttcccatgagctcttggggggagcaggcagagcagctataggttagcagtggccagcaggaggggctggaggggcagggggcagccaaggagctccagcggggtagtcagagcagctataggtgagcaggggccagcgggagggctggaggggcaggggacttccaaggagctccatgggtgcagacagagcagaaactggtgagcaggggccggtgagagatctggagttgcagggggctgcccaggagctccgggggagcaggcagagcagctactggttgcaggggcaggtgggagggctggaggggcaggggcctgcccaggatctccagggagcaggcagagcagctataggtgagcaggggcaggtgagagggctggaggggcagcgggctgcccaggagctccaggggagcaggcagagcagctactggttgcagggcaggtgggagagctggaggggcagcgtcctgcccaggagctccacaggagcagacagagaagctataggtaagcaggggccggtgggagagctgggggggcagggtcctgcccaggagttccgggggagcaggcagagcaggtacaggtgagcaggtgctggtgagaggtctggagaggcagggggctgcccacgactttaggggatgaagggcactgggcctgagttaaacagagccctcaactggtgggacctgagggggtacagggagcagggacagctggaggctcccagggctcaggtcaggctggctgggcagggaggagagttgagctggttgagttgccaggtgcctgttgagttggatggaggggggcccctgggatggaaggagtctagggctgaaggggtcgggggcatccctgagaggaccagatcggatgcagttcttgggtccaggcacaccggccctgttctggggtttgtggggacatgggacaggagggatagacattcggactcagtgtggggtggcacagcgcagtgggctcagtgcatggaagagtaagagtccccttggggcctgagcccaggctaccagcatggggagaggccaggcaaggccctgcggctgagcagacaggactcaggggaggaggggacagggtgggcctggctgttgggggtggaggctgcaccctggcccacacgggggcctggggaggctggcaccgagaggagaggagtgccgagcgcagcgcatggctcccgcccgcgcccaggggccggctgtgctggggaaggaccacagctggaggagggctctgcagtgagcaaggagctggacaggcccaagagcagggagggcagagggcaggcggcgctggcagggaggggccggaggtgagctcaggaagccgggccaggaggacctgagcgggtagggggcccagaggcacatgcaggaggctgggctggggacacacagggatgggaggggagggggacaagaagactcccagggatggggtccagtacacggatggagtccagggaagggacggggtccaggacagggaagggatgggttcaggacagggatggggtcgagggcaaggacaaagcccaaggcatgaacagggttcaagacaggatcaggatcaggggagcgttggggacaagtactcctggtgctgtggctgtggagctgactcagatgacacgtggcctggcgagcagagacaggccatggcaaagtcactgagctgaggggccgggagacaacagggacagtgcggcgcagaggcccaccacacctgaagagatgcccaatcgcccagggctgggccaggctggggccacaggacagcagattagggtccatcagaagccgaccagaagcacaggtctctggtccgggctcagactggggactcagtcaggcgggccacctgcaggaggcctgagcagggacaaggggcaggggccccagggggctctaatctaggttctctgagcctgtggctgcagaggcaccagccctgatgacaggcagccacaaggggcatctaggcctcagactctctggaaacacacgggcgcaggggcaggccctggtggtcacacgcctcctctcttgcag

SEQ ID NO: 4 - DNA, 9313 bp, Sus scrofa
ggtatatatacacactagaataccactcggccgtaaaaacgaaccaaataatgccattggcagcaacacggatggaaccagagactctcatactaagtgaagtaagtcagaaagaccaataccatacgctatcacgtacatctggaatctaatatatggcacaaataaacctttccacagaaaagaaacccatggacttggagaaaatacttgtggtcgccaagggtgaggtagtggggtagactgggagtttggggttagtagatgcaacatattgcatttggcatagataaactatgagatcctgctggagagcacagggagctgtatctagtcccttgtgatggaacatgttggaggataaagtgagaaaaagaacatataaatatatacaccacacgcacacacacacacatatatgaatgactgggtcactttgccgtagagtagaaattgacagaacacagtaaatcaactataacggaaaaaaataaaaaacattaaaaaaaaaaaaacagatgctcatctccatggattctgaacacatttttgacagaattcaacacccaactttttcttgatgatttttattttttccactgcaggtggtttaccctgctctgttgattccactgtacagaaaagtgacaaagtcacacacatatatatatgtagacattctttttctcacattatcctccatcatgccccatcataggtgactagatagagggaaactacaccaacatactaaaagccatatatgacaaacccacaactatcatcattctcaatggtgaaaacctgaaagcatttccgctaagatcaggaacaagacaaggatgtctgctctcaccactctccttccacggagttttggaagtcctagccagggcaattagagaagaaaaggaatccaaatcggaaacggagaagtaacactaccgctctttacaggtgacgtgctgcgatacctagagaatcctcaaggcactaccagaaacctcttagagctcgccaatgaatttggtcaagctgcaggatacaaaattaacacacagaaattggctgccttgctctacgttaacaatgaaagagcagcgagagaaactagggaaaccatcccactgagcagtgcctcgaaatgaatgaaatgcccaggaataaacctacccaaagacacaaagacctggactctgaaaaccataaggcactgctggaagcaaccaaagacgacacaaaccggtggaaagctagaccatgctcctgccctggaagaattaatactggcaaaagggccataccacctaaggccaccctcactctcagtgcaatccctctcaaatctccaatggcattttccacagaactagaacaaaaaacctcaaaaatttgcatggaaacacaaatacctcactgacctgggctatactacaaagctgcagtcatcaaaacggtatggcactgacactcccccccaaaaaagacatacatatcagtggaacagaacagaaagcccagcaataaacccaagcacctatgctcaatccatccatgacataggaggcaagactattcaacagagaagggagagtcacttcactaagtggtgctggggagctggacagctccatgtaaagaatgaaaccagaacactcccaaacgccaaacacaccaaaaaaaaacaaaaaacaaaaaaaaaaaaataaaaaaaccctcaaaacaaatgaaactcctaaacgtaagactggatacctataactcacagaggaaaacaggcagaacgttctttgacataaatcacagcaacatcttatttgatccacctcctggaataactacaataaagacaaaagtggagttcccgtcacggctcagtggtaaccaaatctgactaggaaccatgaggttgcgggttcaatccctggccttgatcagtgggttaaggatccggcgttgccgtcagctgtggtgtaggttgcagacgcggctcggatcccacgttgctgtggctctggtgtaggccgacagctacagctccaattcgacccctagcctgggaacctccacatgccatgggtgcggccctggaaaagacaaaaagacggggaaaaaatgaaagaaagaaagaaggaaagaaggaaaggaggaaagaagaaagaaagaaagaaagaaagaaagaaagaaagaaagaaagacagacagacaaaagtaaaccaatgagatctaattaaactcaaaagcttttgaaaagcaaaagaaaccattttaaaagagaaaaacaggagttcccgtcctggctcaggggttaacgaatctgactagcatccatgaggaggcagatttgatccctggcctcactcagcaggttaaggatccggcattgccatgagctgtgagctgtggtgtaggccagcagccgtagctccaattcaacccctagcccgggaacctccatatgccacaggtggggccctaaaaaccaaaaaagacaaaggaaaacaacccacagaatgggacaaaatctttgcaaacaatgcagcccaccaaggctcaatccccaaaatatacaaacactctacaactcaacaacaacaaaaaacaaaccaacaaacaacccaatcgataaatgggcagaagacctaaatagacatttctccaaagaagacatacagatggccagaagacacatgaaaaaatcctcaacagcactaatttttagggaaatgcaaatcaaaactgcaatgaggcaccacctcacactggtcagaatggccatcattaagagtcaactaacagcaaatgctggagagggtgaggagaagagggaacccgccttcactgttggtgggaatgcaacttggtacaagcactgtggaaaacagtatggaggaacctcagaaaactgaacgtaaaaccacgattgaacccagaaatctcacccctaggcctgtatctcgatacgactttcagtcaaaaacttacatgcacaactatggactttgcagaagggttcaaaatgggcaggacatgaaaacactcaaacgcccatcaacagacgaacggattaagatgtggttctcacacaaagtaagaactacgtctcagccgtaaaaaaggaccaagtcacgccatttgcagcaacgtggatggaactagagactctcacactgagtgaagtaagtcagaaagagaaagacagagaccacatgataccacttatatgtggaatctaaaatacggcgcgattgatcctgtggacaaaacagagacagagcatggtcatggagagcagactttgctttccagggggagggagagaagagattgacagggggtttggagctggtagatgcaactgatcacattgaaaacgagtaagtgatgggtcctactgggcagcacacggaacagtgcccggcctcttgcgttagaacgtgagagaggaaggagagtgtgaaaagaaaaagaaggtaaatgtatgtacagctgtacaagggtaattgaaggaacgttgtaaatcaactctggttttaaaaaaaaaaaaaaaaatttaaagaaagaaaacgttctccagaggcagaacgagaaggtggaacggccgacaggtctctgtcctgcagcggccgctgacacgaccccaggacagcggaaatgagacccaggctgcctgtcgggcacacggcggccgccccggcaccctggtgatcctgtgcagcacgaggtgcccagtagaggggtgggcagggcagggcagggcagggcaggacctggggacgcggccgcagcaggtgggctccagccaggaaggagccacgagtggggtggggtctgctcgctgggctggaggcagggagggcacctcggctccaacatcccagccggggacccggccagaggaaggggctgcgaggatgtcttccaagcatctctttgctcttggaaccacgtggcgaagctttctgaaagcagaccagactgcagcaccacggtttctattgtgaagggatttttccagaaggagtgggaccttgcgaacggggatgtggacacagggtggcttctgatcaagcccagggccctgtgcagccaagtctcatggtcacgccgtcccggccggaccagcctgccccccacgcggacccggggaaggcaggggtcccatgaccagcacgagaacgactgactcgccttgacctcaagccacctccaggaccaggggcagacggcccggcaccacgaggaggaggcagagccccgggcagggcccacttgccacttcacctcgccagagagccgggcagatggcggcacgcgactcttggggcctgacggccccgagcccagacgctctcggggctttggccagactcgcagggctgctgagccaagcagaggctacaaagctccacgggcttccaggcaaccgctagagacacgtgggcacagctcgcagagccggtgacccacgggccagatgggaacctccctcctgtgccccctcaaggcgtctcccggaacggtggcctcccacggcgctggacggggctcggggcaggcccgaggcagcacggcgcccacaccgtccactggccgcttggaaggattcagccaacaccaccccaggcccgctgcacagccggggtgggggctgagacgtccctcaggggtccccagtcccagcctgatgctgaagttacaggacggcccaggggcgcccaggggacagggggagctgcggccctccacgaatgaggacagagggtccctgacccccacgcacggccggcaggtctcggtccacgcagagcagtgagcggctgcccgccagcccgctctcggccgggcaccaccttcacaacccctgggaactctgtccccacgtgggcagcacgggcctgggtctgggcagcaggctgggcccagccacacacgcgggggtcacaagaggtgcccggtggggtcgggcaaccaagggctcacactaggggcctggcacccgggctgcagtaggccaggcggcataatccccgtgggacatagagaaccacgacgtcctgagttcccgcaccctccccaccgctcacccaccccacacacccacatgagccgccagggcactgtctggactcccccaggccctctgggacagaaagcaaccagaagaaaagggaacttcaggaagcagccgggccacccggtttcaatcccattcttagtgtgcagggctgtgggcaggaaggtcgtgcctccagggaccagacacccacccgaggaagcagggctgcaggcacagagcacagacacaaagagccacatgcacctggggccagagcaggaggggcccggagcccggggcagcaggggagccacctcctcggcccccacactcggtgcgtggggttcacgaagaccaggggcaggggcagcggaggggggacgaggaggggagaccccacagcagggctgcagccgacccagcccagggaacacagcctgcacgcgggtcatgtcgcccgcccagcacagggaccagggagccctggcaggggcacgcccacgccaacagaggtggtgccccagaggccagaggaggtcggtggggccgccaggcctctgcggaacatgatgggctggggtcagaggcctgagggatctgctcggccaggttgctggaggcacagggctgggcaggggctgaggacgaggccaggatggacaggtgggtctgggcggaggactcagggaagagacggacacagcgcaggggacaaagaggtcacctggggttgcagggtcctgtcccggctgccggagcccgcttgagccccagggcaggtatgaaagcagtaactgtcgggtcccggggaaggagccagggccacgggcagcaggtgagccgggagcagagcaggcagggcgggccgggctcctgtgtgcccagagctggcagcacagggactgagcctaaagctccacaggccgcaggagggctgacgggagccaggggcctgagggcaggagcgggagccaggggctgggagctgggcgccagtgcagctgagccagcaactagagggagccagggaccagacaggctccaagggtggtcgcggcgagaagagggtgagctcagaagggtcaggagcccctccgggaggggaccaggtgggacagaggctctgccagggggacgctacccagagagggtgggcacccagggctccgggaacaaaggccagccctgggaaagaaaggaagccaggagtggagctagacaaggctgagggttcgggggcaggaagaggggtggtgacactgaggcccaggagccccagggaaaggggcaggtaggctgcaggtgagcagggaaccgggagggcggaaaagctgtccttagagctgctgggaacacatagagctggtgcaggtgaggaggggttgggagggcaagggggctgaacctgaagctcctcggaactggtagagcttgtgcagatgagcaggggctgggagggcagggggcagccctgggaccacctgagagctgtagatcttgggcaggtgagcacaaactggtagggcaggggaagaacctgcagttcctgggagcaggaggagcttgtgcaggtgagcaggggctggagggcagggggctaaacctggagctattgggaagaggttaggcttgtgcaggcgagcagggggtgggagggcgagggtcagtcctgggagccctttcgagccaatagagcccaggcaggtgagcaggggcttggcgggcagggagcatccctgggatgagtttgtgcaggtgagcagggggtgggagtgtagggagcagtcctagcaactcctgggagctggtagagcttgggcaggtgaacaggggctggaagtgcagggggcacatctgggaggccctgggagcaattagcgctctggcaggtgagtaggggctcggagggcagggagcagccctgggagctccttggagcaggtagagcttgggcaggtgagcagggggtgggagggcagggggcagtcctagcaactcctgggagcttgtagagcttgggcaggtgagaacgagctggtagggcaggggaatgaacctggagttactgggtcaggaggagcatgtgcaggtgagcaggggctggagggcagggggctaaacctggagctattgggaagaggttaggcttgtgcaggcaagcagggggtgggagagcaagggtcagtcctgggagcccttgcgagccggtagagcccaggcaggtgagcaggggctgggcgggcagggagcagtcctagcaactcctgggagcaggtagagcttgtgcaggtgagcagggggtgggagggtagggagcagtcctgggaactcctgcgagcaggtagagcttgtgcaggtgggcaggggtgggagggcagggggctgaacctggagctcctcagaactggtagagcttgtgcagatgagcagtggctgggagggcagggggcagccctgggagcaatagaccttgggcaggtgagaacgagctggtagggcagtggaatgaacctggagttactgggtcaagaagagcttgtgcaggcaagcaggggctgaagggcagggggctaaacctggagctattgggaagaggttaggcttgtgcaggcgagcagggggtgggagggcaagggtcagtcctgggagcccttgcgagccggtagagcccaggcaggtgagcaggggctgggagggcagggagcatccctgggacctcttgggagcaggtagagcttgtgcaggtgagcagggggtgggagggtagggagccgtcctagcaactcctgaatctggtagagcttgcggaggtgaacaggggctgggagtgcagggggcagtcctgggagcccctgggagtaattagagcttgggcaggtgagcaggagcttggagtctagggggctaaactggagctattgggaagagtttaggcttgttcaggtgagcaaggggtgggagtagagggggcattcctaggagctcccgcgagttggtcaatctcgggcaggtaagcaggggctcagaggcctggagcagccctgggaccatctgggagcagggcgagcttgggcaggtgagccgggccaggggggatgcaggacagggcaggggcaacagagtgggggtgagctcaggggagccccaggctcgagggaggggccgatatagggctagccaggctggaaagtgggctcctggggggaaggtccccaggactgcgggggcaggggcagggctgaacagacgccatgggtcacagggatcgggccaacgggccataccctgttccagcaaagtgagtggacatgggacagagcagctggagtcctaagtgagaacagcggcacagggcagtgcacatggtcggggggtcgaggggctcatctgggcctgagctggggaagcaggcaatggctgaacagaacaggggggagaggaccggcccaaccgggcaggggcgcatggggtgctggcgccccgtgggagaggaaggccaggggcagggctcggctgcagcagtgctgccaggcaggctggcagcgggaagggcaggagcagagggagggctctggcctcagcaggcagctgggaggcccagagctgggctgcaggggccggggctctggggaaagctgcctcgggtgagctcagggccaagccaggagccccggtggaggagggaccggtgggcccagaggcccagatttggagcctgggccagggccggagggatggcaggggaggggcctcggaggagactacctgccctcgcccagggaaaggggcatgttggtgccagggaccaacgcgcacgggccttgactgcagagctgacccagaggaacggggcctggagagcaaacagcaggccaggaccaggggccaggagcagagcatgggcagtgggtggcagtgggtggcagtgtccctgccaggctggggaagcaggacggtggcctcaggggaccagcagaagctggccaggaacacaggccacccgctggtcgggccaggcacccacaggggctctgggccctggcgggctctcatccaggcggtcacccagctctgaccaggcagctgcgcccagaacctgcagctgcaatggcagcgagctgggcggctggtctgccgactttctggaagcaagtgggtgctgggcgcagcggccccgctgttctgagggcccgattcgctgcccgccccacaaggaacaaggccctggcggtcacgcagcctcctctcttccag

SEQ ID NO: 5 - DNA, 337 bp, Bos taurus
tccactagggtggccaggcacagtcaccagagggggagggctcgggtgcaggggtgcgggagggtgggggaggcagcggtgtttgtgtcctcttgtttttctctttcttctcaagccccctgcacctcatcacctgctgaaacatccaaaatagctctaggtggctactgagtcattgcgagcacagcccaacccaggtgtcccagccaggctgctcttctgagaatcgggccccaaaaccgagacctggccaggtgggcctggggcctgggcccggggccaaagcccaggggagtcctacgggggcagtgagttccccaaggcctggagagggccc

SEQ ID NO: 6 - DNA, 246 bp, Bos taurus
ccccaccccctaggcaggtgcgaggccctctcagtttcccccaggttactcatttggggcacactcagccttgcagggcatgcaaatggctgtttgttccacactgaaaaacatgtctaagcctctgtggttatttccagaaatagcctacgcccacgccccacctgcagccccagctctgaccctccagagtgccaggctggcctggagctcaggattcgggggaccctgcaccccctgccccag

SEQ ID NO: 7 - protein, 145 aa, Homo sapiens
MSWAPVLLMLFVYCTGCGPQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSEKEEREREWEEEMEPTAARTRVP

SEQ ID NO: 8 - protein, 123 aa, Homo sapiens
MACRCLSFLLMGTFLSVSQTVLAQLDALLVFPGQVAQLSCTLSPQHVTIRDYGVSWYQQRAGSAPRYLLYYRSEEDHHRPADIPDRFSAAKDEAHNACVLTISPVQPEDDADYYCSVGYGFSP

SEQ ID NO: 9 - DNA, 177 bp, Homo sapiens
gtcacatctggactcccccaggccctctgagacagaaaacactcagaagaaaagggaacttcaggaagcaagtcgcgtcaccaggtttcattcctgttcttagtcttcacagcactgggggaagggccctcacgcctcctgggactggtgaccaagtcccagggagcagggctgcag

SEQ ID NO: 10 - protein, 476 aa, Bos taurus
EGESHPRVFPLVSCVSSPSDESTVALGCLARDFVPNSVSFSWKFNNSTVSSERFWTFPEVLRDGLWSASSQVVLPSSSAFQGPDDYLVCEVQHPKGGKTVGTVRVIATKAEVLSPVVSVFVPPRNSLSGDGNSKSSLICQATDFSPKQISLSWFRDGKRIVSGISEGQVETVQSSPITFRAYSMLTITERDWLSQNVYTCQVEHNKETFQKNVSSSCDVAPPSPIGVFTIPPSFADIFLTKSAKLSCLVTNLASYDGLNISWSRQNGKALETHTYFERHLNDTFSARGEASVCSEDWESGEEFTCTVAHSDLPFPEKNAVSKPKDVAMKPPSVYLLPPTREQLSLRESASVTCLVKGFAPADVFVQWLQRGEPVTKSKYVTSSPAPEPQDPSVYFVHSILTVAEEDWSKGETYTCVVGHEALPHMVTERTVDKSTEGEVSAEEEGFENLNTMASTFIVLFLLSLFYSTTVTLFKVK

SEQ ID NO: 11 - protein, 471 aa, Equus ferus
ESTKTPDLFPLVSCGPSLDESLVAVGCLARDFLPNVITFSWNYQNNTVVRSQDIKNFPSVLREGKYTASSQVLLPSGDVPLVCTVNHSNGNKKVEVRPQVLIQDESPNVTVFIPPRDAFTGPGQRTSRLVCQATGFSPKEISVSWLRDGKPVESGFTTEEVQPQNKESWPVTYKVTSMLTITESDWLNQKVFTCHVEHQQGVFQKNVSSMCSPNSPVPIKIFAIPPSFAGIFLTKSAKLSCQVTNLGTYDSLSISWTRQNGEILKTHTNISESHPNGTFSALGEATICVEDWESGDDYICTVTHTDLPFPLKQAISRPDAVAKHPPSVYVLPPTREQLSLRESASVTCLVKGFSPPDVFVQWLQKGQPLSSDKYVTSAPMPEPQAPGLYFVHSILTVSEEDWSSGETYTCVVGHEALPHVVTERTVDKSTEGEVSAEEEGFENLSAMASTFIVLFLLSLFYSTTVTLFKVK

SEQ ID NO: 12 - protein, 476 aa, Ovis aries
ESESHPKVFPLVSCVSSPSDENTVALGCLARDFVPNSVSFSWKFNNSTVSSERFWTFPEVLRDGLWSASSQVALHSSSTFQGTDGYLVCEVQHPKGGKTVGTVMVVAPKVEVLSPVVSVFVPPCNSLSGNGNSKSSLICQATDFSPKQISLSWFRDGKRIVSDISEGQVETVQSSPTTYRAYSVLTITEREWLSQSAYTCQVEHNKETFQKNASSSCDATPPSPIGVFTIPPSFADIFLTKSAKLSCLVTNLASYDGLNISWSHQNGKALETHTYFERHLNDTFSARGEASVCSEDWESGEEYTCTVAHLDLPFPEKSAISKPKDVAMKPPSVYVLPPTREQLSLRESASVTCLVKGFAPADVFVQWLQKGEPVAKSKYVTSSPAPEPQDPSAYFVHSILTVTEEDWSKGETYTCVVGHEALPHMVTERTVDKSTEGEVSAEEEGFENLNTMASTFIVLFLLSLFYSTTVTLFKVK

SEQ ID NO: 13 - protein, 477 aa, Sus scrofa
ESQSAPNLFPLVSCVSPPSDESLVALGCLARDFLPSSVTFSWNYKNSSKVSSQNIQDFPSVLRGGKYLASSRVLLPSVSIPQDPEAFLVCEVQHPSGTKSVSISGPVVEEQPPVLNIFVPTRESFSSTPQRTSKLICQASDFSPKQISMAWFRDGKRVVSGVSTGPVETLQSSPVTYRLHSMLTVTESEWLSQSVFTCQVEHKGLNYEKNASSLCTSNPNSPITVFAIAPSFAGIFLTKSAKLSCLVTGLVTRESLNISWTRQDGEVLKTSIVFSEIYANGTFGARGEASVCVEDWESGDRFTCTVTHTDLPSPLKQSVSKPRGIARHMPSVYVLPPAPEELSLQEWASVTCLVKGFSPADVFVQWLQKGEPVSADKYVTSAPVPEPEPKAPASYFVQSVLTVSAKDWSDGETYTCVVGHEALPHTVTERTVDKSTEGEVSAEEEGFENLNTMASTFIVLFLLSLFYSTTVTLFKVK

SEQ ID NO: 14 - protein, 471 aa, Artificial Sequence
  chimeric IgM
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVETVQSSPITFRAYSMLTITERDWLSQNVYTCQVEHNKETFQKNVSSSCDVAPPSPIGVFTIPPSFADIFLTKSAKLSCLVTNLASYDGLNISWSRQNGKALETHTYFERHLNDTFSARGEASVCSEDWESGEEFTCTVAHSDLPFPEKNAVSKPKDVAMKPPSVYLLPPTREQLSLRESASVTCLVKGFAPADVFVQWLQRGEPVTKSKYVTSSPAPEPQDPSVYFVHSILTVAEEDWSKGETYTCVVGHEALPHMVTERTVDKSTEGEVSAEEEGFENLNTMASTFIVLFLLSLFYSTTVTLFKVK

SEQ ID NO: 15 - protein, 220 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 16 - protein, 256 aa, Artificial Sequence
  Made in Lab construct
MKWVTGISLLFLFSSSRAHHHHHHHHHLEVLFQGPFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERT

SEQ ID NO: 17 - protein, 257 aa, Artificial Sequence
  Made in Lab construct
METDTLLLWVLLLWVPGSTGHHHHHHHHHHENLYFQGFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER